(19) United States
(12) Patent Application Publication
(10) Pub. No.: US 2008/0268045 A1
(43) Pub. Date: Oct. 30, 2008

(54) SUSTAINED RELEASE METHOTREXATE FORMULATIONS AND METHODS OF USE THEREOF

(75) Inventors: Thierry Dervieux, San Diego, CA (US); Kay Olmstead, San Diego, CA (US)

Correspondence Address: WILSON SONSINI GOODRICH & ROSATI, 650 PAGE MILL ROAD, PALO ALTO, CA, 94304-1050, US

(73) Assignee: Proprius Pharmaceuticals, Inc., San Diego, CA (US)

(21) Appl. No.: 11/937,953

(22) Filed: Nov. 9, 2007

Related U.S. Application Data
(60) Provisional application No. 60/858,220, filed on Nov. 9, 2006.
(60) Provisional application No. 60/913,501, filed on Apr. 23, 2007.

(51) Int. Cl. A61K 31/4985; A61P 37/00; A61P 35/00; A61P 35/02; A61K 9/22

(52) U.S. Cl. 424/468; 514/249

(57) ABSTRACT

Described herein are methods of treating a disease by treatment with oral sustained release methotrexate alone or in combination with folates. In some embodiments, these approaches improve the pharmacotherapeutic performance of methotrexate therapy.

Described herein are novel pharmaceutical compositions for oral administration. Also described herein are novel pharmaceutical compositions for the controlled, sustained delivery of one or more drugs to the stomach or upper gastrointestinal tract. Further described are novel pharmaceutical compositions with increased gastrointestinal residence time. More particularly, novel pharmaceutical compositions which can simultaneously, float in gastric fluid, adhere to the mucosal surfaces of the gastrointestinal tract, swell to a size which delays passage through the pylorus, are described herein. In some embodiments, the pharmaceutical compositions comprise methotrexate. In some embodiments, the pharmaceutical compositions comprise methotrexate and a folate compound. Also described herein are methods for treating or preventing diseases, by administration of the pharmaceutical compositions described herein.

### CROSS REFERENCE

This application claims the benefit of U.S. Provisional Application No. 60/858,220, filed Nov. 9, 2006, and U.S. Provisional Application No. 60/913,501, filed Apr. 23, 2007, both of which are incorporated herein by reference in their entirety.

### BACKGROUND OF THE INVENTION

Rheumatoid arthritis (RA) is a chronic inflammatory disorder with systemic features and joint involvement that results in an erosive synovitis, cartilage degradation and joint destruction. Structural damage to the joints is predictive of long-term outcome and contributes to functional decline, disability and the need for major surgery. This progressive, chronic, and often crippling disease usually starts in middle age but may also occur in children and young adults,

Current treatments for RA are focused on treating symptoms (e.g. joint pain, stiffness and swelling) and the underlying disease process. Treatments for RA symptoms include corticosteroids (e.g., prednisone) and nonsteroidal anti-inflammatory drugs (NSAIDs) (e.g., aspirin, ibuprofen, indomethacin, naproxen). Compounds called disease modifying antirheumatic drugs (DMARDs) (e.g., methotrexate, azathioprine, hydroxychloroquine, cyclosporine, D-penicillamine, sulfasalazine, leflunomide and minocycline) and genetically engineered monoclonal antibody-based drugs (e.g., infliximab, etanercept, adalimumab) are targeted to causative factors of RA. The antibody-based drugs target and neutralize an inflammation-causing protein called tumor necrosis factor-α (TNFα).

For the past 20 years, the DMARD of choice has been low-dose methotrexate. However, there are significant disadvantages connected with methotrexate therapy. While effective at controlling disease activity and decreasing functional disability in a significant subset of patients with RA, methotrexate is ineffective in approximately 40% of individuals. Another major drawback with methotrexate is the unpredictable appearance of a large spectrum of side effects (Weisman et al., Arthritis Rheum, 2006, 54, 607-612; Dervieux et al., Arthritis Rheum, 2006, 54, 3095-3103) that include gastrointestinal distress (vomiting, nausea), stomatitis, headache, alopecia elevation of liver enzymes and, less frequently, hematological toxicities and pulmonary infiltrates.

It is believed that methotrexate enters the cells through the Reduced Folate Carrier and is intracellularly converted to methotrexate polyglutamates (MTXPGs) by a γ-linked sequential addition of glutamic acid residues on methotrexate (Chabner et al., J Clin Invest, 1985, 76, 907-912). This process of polyglutamation enhances the intracellular retention of methotrexate, promotes the sustained inhibition of de novo purine synthesis (AICAR transformylase) and the build-up of adenosine, a potent anti-inflammatory agent (Cronstein et al., J. Clin. Invest., 1993, 92, 2675-2682; Morabito et al., J. Clin. Invest., 1998, 101, 295-300; Dervieux et al., Blood, 2002, 100, 1240-1247). Methotrexate also directly inhibits several other folate dependent enzymes including Dihydrofolate Reductase (DHFR), Thymidylate Synthase (TS) and AICAR transformylase (ATIC) (Allegra et al., J. Biol. Chem., 1985, 260, 9720-9726; Allegra et al., Proc. Natl. Acad. Sci., 1985, 82, 4881-4885) (FIG. 1). Other folate dependent enzymes such as Methylenetetrahydrofolate Reductase (MTHFR) or Methionine Synthase (MS) are not directly inhibited by methotrexate but their expression levels contribute to the anti-folate effects (Kremer, Arthritis Rheum., 2004, 50, 1370-1382; Dervieux et al., Ann. Rheum. Dis., 2005, 64, 1180-1185; Weisman et al., Arthritis Rheum, 2006).

While MTXPG accumulation and de novo purine synthesis inhibition are associated with methotrexate efficacy and therapeutic response (Masson et al., J. Clin. Invest., 1996, 97, 73-80; Dervieux et al., Arthritis Rheum., 2004, 50, 2766-2774), DHFR inhibition and depletion in folate pools account for most of methotrexate's induced idiosyncrasies. This is supported by the observation that low folate status is a risk factor for methotrexate's induced adverse events (Morgan et al., J. Rheumatol., 1998, 25, 441-446) and by evidence suggesting that folic acid or folinic acid supplementation can reduce methotrexate toxicities (Morgan et al., Ann. Intern. Med., 1994, 121, 833-841; Ortiz et al., J. Rheumatol., 1998, 25, 36-43; Morgan et al., Arthritis Rheum., 2004, 50, 3104-3111). Methotrexate therapy is also associated with transient elevation in homocysteine levels (Kishi et al., J. Clin. Oncol., 2003, 21:3084-3091; Hoekstra et al., Ann. Rheum. Dis., 2005, 64, 141-143) and the pathophysiological mechanism associated with methotrexate's induced mild hyperhomocysteinemia results from depletion in 5-methyltetrahydrofolate, the cofactor for remethylation of homocysteine to methionine by MS. Folate supplementation can prevent the elevation in homocysteine levels and therefore may decrease the risk for hyperhomocysteinemia mediated toxicities (Haagsma et al., Ann. Rheum. Dis., 1999, 58, 79-84). Depletion in intracellular folate stores may be an important determinant of therapeutic response to methotrexate in rheumatoid arthritis patients.

Methotrexate is widely used in the treatment of patients with autoimmune diseases including, but not limited to, rheumatoid arthritis, ankylosing spondylitis, Crohn's disease, juvenile rheumatoid arthritis, psoriatic arthritis, psoriasis, scleroderma, polymyositis, systemic lupus erythematosus and vasculitis; it is also used to treat cancers such as acute lymphocytic leukemia, meningeal leukemia, choriocarcinoma, osteosarcoma, cutaneous lymphoma, Burkitt's lymphoma, non-Hodgkin's lymphoma, breast cancer, head and neck cancer, ovarian cancer and bladder cancer.

Dihydrofolate reductase inhibition and depletion in folate pools have been associated with the side effects observed in patients receiving methotrexate. Methotrexate therapy is also associated with transient elevation in homocysteine levels and the pathophysiological mechanism associated with this methotrexate induced mild hyperhomocysteinemia results from depletion in 5-methyltetrahydrofolate.

Transient hyperhomocysteinenia is a common feature associated with oral low dose weekly methotrexate therapy (Hoekstra et al., 2005 Ann Rheum. Dis., 64:141-143). Following oral administration, this elevation usually peaks between 8 and 48 hours and is contemporary to the appearance of acute side effects.

Rheumatoid arthritis patients receiving low dose weekly methotrexate are more likely to discontinue treatment because of toxicity rather than because of lack of efficacy (Alarcon et al., 1995 Ann Rheum Dis 54:708-12). In fact, the appearance of toxic signs in the first 24 hours following methotrexate administration often precludes a dosage escalation in the dose range likely to maximize the therapeutic effects. Supplementation with folate can decrease methotrexate toxicities (Morgan et al., 1994 Ann Intern Med 121:833-841; Ortiz et al., 1998 J Rheumatol 25:36-43; van Ede et al., 2001 Arthritis Rheum 44:1515-1524; Morgan et al., 2004 Arthritis Rheum 50:3104-3111). However, the particular folate to be administered, as well as the optimal dosing and schedule of administration (daily vs. weekly), are controversial. Folic acid administration has consistently resulted in a decrease in the incidence of adverse events caused by methotrexate without a significant effect on efficacy (Ortiz et al., 1998 J Rheumatol 25:36-43).

### SUMMARY OF THE INVENTION

Described herein are methods of treating an autoimmune disease by oral administration of a sustained release methotrexate composition. The sustained release decreases the incidence of acute side effects occurring in the first 48 hours following administration of the drug, and results in increased efficacy.

In various embodiments of the invention described herein, the administration of 5-methyltetrahydrofolate in combination with sustained release methotrexate will decrease methotrexate toxicity without affecting efficacy. Without wishing to be bound by any particular theory, this improvement in the benefit-risk ratio profile is based on the direct supplementation of the folate species ensuring the remethylation of homocysteine to methionine (FIG. 1) (Lamers et al., 2004 Am J. Clin Nutr 79:473-478). In contrast, folic acid is first converted to 5-methyltetrahydrofolate by a multistep process, and a decrease in the expression level of MTHFR (as seen in those with MTHFR 677TT genotype) results in accumulation of 5, 10 CH2-THF which can result in decreased efficacy for methotrexate as recently shown in vitro (Sohn et al., 2004 J Natl Cancer Inst 96:134-144) and in vivo in patients with RA (Dervieux et al., 2006 Arthritis Rheum 54:3095-3103).

Also described herein are novel pharmaceutical compositions for oral administration. Also described herein are novel pharmaceutical compositions for the controlled, sustained delivery of one or more active agents to the stomach or upper gastrointestinal tract. Further described are novel pharmaceutical compositions with increased gastrointestinal residence time. More particularly, novel pharmaceutical compositions which can simultaneously, float in gastric fluid, adhere to the mucosal surfaces of the gastrointestinal tract, swell to a size which delays passage through the pylorus, are described herein. Also described are methods of treating diseases by administration of the pharmaceutical compositions described herein. Provided herein are gastric-retentive dosage forms which release active agents in a controlled manner through incorporation of one or more agents that collectively float in gastric fluid, adhere to the mucosal surfaces of the gastrointestinal tract, and/or swell to a size which delays passage through the pylorus. In some embodiments, these compositions form microgels upon exposure to gastric fluid.

Provided herein are methods for treating an autoimmune disease in a subject comprising administering to the subject an oral pharmaceutical composition comprising methotrexate, which releases the methotrexate into the upper gastrointestinal tract of the subject over a sustained period of time. In some embodiments, half of the total systemic methotrexate AUC is delivered between about 4 and about 24 hours. In some embodiments, the oral pharmaceutical composition is a monolithic solid tablet. In some embodiments, the autoimmune disease is selected from ankylosing spondylitis, Crohn's disease, rheumatoid arthritis, juvenile rheumatoid arthritis, psoriatic arthritis, psoriasis, scleroderma, polymyositis, lupus, systemic lupus erythematosus, vasculitis, inflammatory bowel disease, Sjogren's syndrome and multiple sclerosis. In some embodiments, the autoimmune disease is rheumatoid arthritis.

Provided herein are methods for treating or preventing cancer in a subject comprising administering to the subject an oral pharmaceutical composition comprising methotrexate, which releases the methotrexate into the upper gastrointestinal tract of the subject over a sustained period of time. In some embodiments, half of the total systemic methotrexate AUC is delivered between about 4 and about 24 hours. In some embodiments, the oral pharmaceutical composition is a monolithic solid tablet. In some embodiments, the cancer is selected from acute lymphocytic leukemia, meningeal leukemia, choriocarcinoma, osteosarcoma, cutaneous lymphoma, Burkitt's lymphoma, non-Hodgkin's lymphoma, breast cancer, head and neck cancer, ovarian cancer and bladder cancer.

Provided herein are methods for optimizing the tolerability of methotrexate for treatment of an autoimmune disease in a subject comprising administering to the subject an oral pharmaceutical composition comprising methotrexate, which releases the methotrexate into the upper gastrointestinal tract of the subject over a sustained period of time. In some embodiments, half of the total systemic methotrexate AUC is delivered between about 4 and about 24 hours. In some embodiments, the oral pharmaceutical composition is a monolithic solid tablet. In some embodiments, the methotrexate in the sustained release formulation is at least about 10% more bioavailable than Trexall® or Rheumatrex. In some embodiments, the optimization of tolerability is a decrease in Cₘₐₓ. In some embodiments, the optimization of tolerability is an increase in bioavailability. In some embodiments, the increase in bioavailability of the sustained release dosage form as compared to an immediate release dosage form is at least 10%. In some embodiments, the increase in bioavailability of the sustained release dosage form as compared to an immediate release dosage form is at least 20%. In some embodiments, the bioavailability is determined across a group of patients. In various embodiments, the group of patients consists of 6, 12, 24, 50, 100 or more than 100 patients.

Provided herein are methods for reducing the toxicity of methotrexate for treatment of an autoimmune disease in a subject comprising administering to the subject an oral pharmaceutical composition comprising methotrexate, which releases the methotrexate into the upper gastrointestinal tract of the subject over a sustained period of time. In some embodiments, half of the total systemic methotrexate AUC is delivered between about 4 and about 24 hours. In some embodiments, the oral pharmaceutical composition is a monolithic solid tablet.

Provided herein are methods for improving the risk/benefit ratio of methotrexate for treatment of an autoimmune disease in a subject comprising administering to the subject an oral pharmaceutical composition comprising methotrexate, which releases the methotrexate into the upper gastrointestinal tract of the subject over a sustained period of time. In some embodiments, half of the total systemic methotrexate AUC is delivered between about 4 and about 24 hours. In some embodiments, the oral pharmaceutical composition is a monolithic solid tablet.

Provided herein are methods of treatment comprising providing a pharmaceutical compositions for oral delivery comprising methotrexate, which releases said methotrexate into the upper gastrointestinal tract over a sustained period of time; and a composition comprising a folate. In some embodiments, half of the total systemic methotrexate AUC is delivered between about 4 and about 24 hours. In some embodiments, the oral pharmaceutical composition is a monolithic solid tablet. In some embodiments the composition comprising methotrexate and the composition comprising the folate are the same composition. In other embodiments, the composition comprising the methotrexate and the composition comprising the folate are different compositions which are part of a kit.

In various embodiments, the compositions described herein have an advantage that they may be retained for long periods of time in the stomach. In some embodiments, the active agent is administered daily. In other embodiments, the active agent is administered weekly or bi-weekly. By releasing an active agent in a controlled manner over a period of time, this dosage form can reduce undesirable side effects or toxic effects of certain drugs. Also, the compositions described herein have the advantage that they provide gastric retention in order to improve the absorption of the active agents which have specific absorption sites between the stomach and the jejunum.

In some embodiments, the dosage forms of the present invention offer benefits to highly soluble drugs whose delivery from the matrix occurs primarily by diffusion out of the matrix after being dissolved by gastric fluid. The dosage forms of the present invention also offer benefits to sparingly soluble drugs whose delivery from the matrix occurs primarily by erosion of the matrix. In addition, the dosage forms of the present invention find utility when administered to subjects who are in either the fed state or the fasting state. In some embodiments, administration during the fed state is preferred, since the narrowing of the pyloric opening that occurs in the fed state serves as a further means of promoting gastric retention by retaining a broader size range of the dosage forms. The fed state is normally induced by food ingestion, but can also be induced pharmacologically by the administration of pharmacological agents, known to those of skill in the art, that have an effect that is the same or similar to that of a meal. These fed-state inducing agents may be administered separately or they may be included in the dosage forms described herein as an ingredient dispersed in the dosage form or in an outer immediate release coating.

In one embodiment of the present invention the active agent for controlled delivery may exhibit a small absorption window in the gastrointestinal tract. In some embodiments of the present invention, the small absorption window is in the duodenum.

In some embodiments, solid pharmaceutical compositions for sustained-release of one or more active agents into the upper gastrointestinal tract, comprising: (i) at least one active agent; (ii) an agent that floats in gastric fluid; (iii) a water-swellable, gelling agent; and (iv) a bioadhesive agent are described.

In other embodiments, controlled-release oral drug dosage forms comprising at least one drug dispersed in at least one solid polymeric matrix, wherein said polymeric matrix (i) swells and gels upon imbibition of water; (ii) floats in gastric fluid; and (iii) adheres to the mucosal surfaces of the gastrointestinal tract are described.

In yet other embodiments, solid monolithic pharmaceutical compositions for controlled, sustained-release of an active agent into the stomach comprising: (i) at least one active agent; (ii) at least one floating agent; and (iii) at least one water-swellable, gel forming agent; wherein, upon oral administration to a subject: (a) said gas generating agent reacts with gastric fluid to generate gas; (b) said water-swellable, gel forming agent swells and entraps the gas generated by said gas generating agent, to produce a dosage form of increased size which floats in the gastric environment; and (c) said dosage form of increased size is of a size that promotes retention in the stomach are described. In various embodiments, the agent located at the outer surface of said dosage form of increased size is dissolved in gastric fluid and released via a leaching action, wherein only the outer layer of the tablet incorporating the active agent contacts the gastric mucosa. In various embodiments, the dosage form of increased size maintains its physical integrity for at least a substantial portion of the time period during which the drug is released into the stomach.

In some embodiments, controlled release solid pharmaceutical compositions for oral administration comprising (i) at least one water-swellable polymer; (ii) at least one active agent dispersed in said water-swellable polymer; and (iii) at least one gas generating agent dispersed in said water-swellable polymer; wherein, upon oral ingestion and subsequent contact with gastric fluid: (a) said gas generating agent reacts with gastric fluid to generate gas; (b) said water-swellable polymer swells uniformly wherein said swelling entraps the gas generated by said gas generating agent, to produce a dosage form of increased size which (i) floats in the gastric environment and (ii) promotes gastric retention in the stomach; (c) said dosage form of increased size releases said drug to the stomach, as a result of said erosion at a substantially constant rate, wherein said rate maintains said active agent at a therapeutically effective concentration are described. In some embodiments, the dosage form gradually erodes. In various embodiments, the erosion does not substantially reduce the over-all physical integrity of said dosage form of increased size during a substantial portion of the time period during which the drug is released into the stomach.

Provided herein are oral pharmaceutical compositions comprising methotrexate, wherein the pharmaceutical composition releases the methotrexate into the upper gastrointestinal tract of a subject over a sustained period of time. In some embodiments, half of the total systemic methotrexate AUC is delivered between about 4 and about 24 hours. In some embodiments, the pharmaceutical composition is a monolithic solid tablet form.

Provided herein are solid pharmaceutical compositions for sustained-release of methotrexate into the stomach, comprising: (i) methotrexate; and (ii) carbopol.

Provide herein are solid pharmaceutical composition for sustained-release of methotrexate into the stomach, comprising: (i) methotrexate; (ii) carbopol; and (ii) a gas generating agent.

Also described herein are solid pharmaceutical composition for sustained-release of methotrexate into the stomach, comprising: (i) methotrexate; (ii) a folic acid derivative; and (ii) carbopol. In addition, described herein are solid pharmaceutical composition for sustained-release of methotrexate into the stomach, comprising: (i) methotrexate; and (ii) a cellulose derivative; solid pharmaceutical composition for sustained-release of methotrexate into the stomach, comprising: (i) methotrexate; (ii) a cellulose derivative; and (ii) a gas generating agent; solid pharmaceutical composition for sustained-release of methotrexate into the stomach, comprising: (i) methotrexate; (ii) a folic acid derivative; and (ii) a cellulose derivative; and solid pharmaceutical composition for sustained-release of methotrexate into the stomach, comprising: (i) methotrexate; (ii) at least one cellulose derivative; and (ii) carbopol. In some embodiments the methotrexate is present in an amount of 1-25 mgs or about 1 mg, about 5 mgs, about 7.5 mgs, or about 15 mgs.

In some embodiments, the methotrexate comprises from about 0.5% to about 5% by weight of the composition. In other embodiments, the methotrexate comprises from about 1% to about 3% by weight of the composition.

In some embodiments, the methotrexate comprises about 1 mg to about 20 mg of the composition. In other embodiments, the methotrexate comprises from about 2 mg to about 15 mg of the composition.

In some embodiments, the composition further comprises a hydrophilic polymer. In some embodiments, the hydrophilic polymer comprises carbopol, hydroxypropyl cellulose, hydroxymethyl cellulose, polyethylene oxide, or mixtures thereof. In some embodiments, the hydrophilic polymer is carbopol.

In some embodiments, the carbopol comprises from about 15% to about 40% by weight of the composition. In other embodiments, the carbopol comprises from about 20% to about 40% by weight of the composition. In other embodiments, the carbopol comprises from about 20% to about 30% by weight of the composition. In various embodiments the carbopol comprises from about 20% to about 30% by weight of the composition and the methotrexate comprises from about 2 mg to about 15 mg of the composition.

In some embodiments, the composition further comprises at least one diluent. In some embodiments, the diluent is lactose, a lactose derivative, dicalcium phosphate, a microcrystalline cellulose, a compound product of microcrystalline cellulose, silicon dioxide, a starch, a starch derivative, a pregelatinized starch or a combination thereof. In various embodiments, the diluent comprises about 10% to about 80% by weight of the composition.

In some embodiments of the compositions described herein the composition comprises an agent that floats in gastric fluid. In some embodiments, that agent has a specific gravity lower that that of gastric fluid. In other embodiments, that agent has at least one gas generating agent. In these embodiments, the gas may or may not become entrapped within said pharmaceutical composition.

In some embodiments, the composition further comprises at least one gas generating agent. The gas generating agent can be comprised of different components (such as a carbonate or bicarbonate) and vary in amounts. In some embodiments, it is present in an amount of between about 5-30 wt-%. In various embodiments, the gas generating agent is a carbonate or bicarbonate salt of a Group I and Group II metal. In some embodiments, the gas generating agent is sodium bicarbonate. In some embodiments, the gas generating agent makes up about 3 wt-% to about 20 wt-% of the composition.

In some embodiments, the pharmaceutical composition further comprises at least one bioadhesive agent. In some embodiments, the bioadhesive agent is Carbopol, Polycarbophil, a natural gum, guar gum, xanthan gum, chitosan, a chitosan derivative, 5-methyl-pyrrolidone chitosan (MPC), hydroxypropyl cellulose (HPC), Klucel, hydroxypropyl methylcellulose (HPMC), sodium carboxymethyl cellulose, a copolymer of acrylic acid, a copolymer of methacrylic acid, a poly(methyl vinyl ether/maleic anhydride) copolymer, pectin, alginic acid, a salt of alginic acid, sodium alginate, hyaluronic acid, gum tragacanth or karaya gum or combinations thereof. In some embodiments, the bioadhesive agent is carbopol.

In some embodiments, the composition comprises at least one gelling agent. In some embodiments, the gelling agent is a swellable, gelling agent. The swellable, gelling agents useful in the composition described herein can cause the dosage form to swell to about twice the original size, or about three times the original size or about four times the original size, as measured along the largest dimension of the dosage form. In some embodiments, the swellable, gelling agent forms discrete microgels upon exposure to water, which can be soluble or insoluble in water. In various embodiments, the internal osmotic pressure within said microgels results in slow sloughing off of discrete pieces of the microgel, thereby resulting in release of drug. In some embodiments, the gelling agent is carbopol, polycarbophil, hydroxypropyl methylcellulose (HPMC), methylcellulose, hydroxypropyl cellulose (HPC), carbomer, carboxy methylcellulose, gum tragacanth, gum acacia, guar gum, pectin, a modified starch derivative, xanthan gum, locust bean gum, chitosan, a chitosan derivative, sodium alginate, polyvinyl acetate (Kollidon-SR), polyethylene oxide or polyoxide or a combination thereof. In some embodiments the composition comprises carbopol as the gelling agent.

Depending on the embodiment, the agent that floats in gastric fluid, said water-swellable, gelling agent and said bioadhesive agent comprise the same compound, and/or the agent that floats in gastric fluid, said water-swellable, gelling agent and said bioadhesive agent each comprise a cross linked polyacrylic acid polymer. In some embodiments, these agents comprise carbopol.

In some embodiments the composition comprises one or more adsorbents, fillers, antioxidants, buffering agents, colorants, flavorants, sweetening agents, antiadherents, lubricants, glidants, binders, diluents, disintegrants, tablet direct compression excipients or polishing agents.

In some embodiments, the composition has a hardness of between about 6-15 Kp. In some embodiments, the composition has a friability less than 1%. In some embodiments, the composition has a friability less than 0.5%. In some embodiments, the composition has a hardness of between about 6-15 Kp and a friability less than 0.5%.

In some embodiments, the solid monolithic form is tetrahedral in shape. In some embodiments, the solid monolithic form is a diamond shaped tablet with a triangular lateral length of 6-11 mm and a vertical axis length of about 2-7 mm. In some embodiments, the triangular lateral length is about 8-10 mm. In some embodiments, the vertical axis length is about 3-5 mm. In some embodiments, the monolithic solid tablet is a diamond or triagonal biplanar shaped tablet with a triangular lateral length of 8-14 mm and a vertical axis length of about 4-9 mm.

In some embodiments, the composition adheres to the mucosal surface of the gastrointestinal tract. In some embodiments, the composition adheres to intestinal tissue. In some embodiments, the composition adheres to porcine stomach tissue. In various embodiments, the composition adheres to intestinal tissue with a force of at least 500,000 nJ. In some embodiments, the composition adheres to intestinal tissue with a force greater than that of Glumetza® (500 mg metformin/tablet).

In various embodiments, the pharmaceutical compositions of the present invention are designed to release about 50% of said drug between about 4 and about 10 hours after immersion, or about 75% of said drug between about 3 and about 12 hours after immersion. In some embodiments, the composition of the present invention retains at least about 50% of said drug 7 hours after immersion; releases 75% of said drug within about 15 hours after immersion, and releases substantially all of said drug within about 24 hours after immersion.

In some embodiments, upon immersion of the composition in gastric fluid, the composition swells in size. In some embodiments, the composition swells in size to a diameter of at least 15 mm within 2 hours. In some embodiments, the composition swells in size to a diameter of at least 13 mm within 1 hour. In some embodiments, the composition swells in size by at least 25% in all measurements within 1 hour and at least 50% in length and width within 9 hours. In some embodiments, upon immersion in simulated gastric fluid at pH 1.2 and at 37° C., the pharmaceutical composition swells in size by at least about 25% in all measurements within about 1 hour. In some embodiments, the composition swells in size by at least about 50% in at least one measurement within about 1 hour and about 9 hours after immersion in the simulated gastric fluid.

In some embodiments, the composition floats in gastric fluid. In some embodiments, the composition floats in simulated gastric fluid (SGF; pH 1.2, no enzyme) within 20 minutes after immersion and remains floating for at least 4 hours. In some embodiments the composition remains floating for at least 6 hours. In some embodiments, the composition remains floating for at least 8 hours. In some embodiments, the composition remains floating for about 4-12 hours. In some embodiments, the composition remains floating for about 4-10 hours. In some embodiments, the composition remains floating for about 6-12 hours. In other embodiments, the composition remains floating for about 6-10 hours.

In some embodiments, the composition exhibits a methotrexate dissolution rate (measured by the USP Type II dissolution apparatus (paddle method) at 100 rpm in 900 ml 0.1N hydrochloric acid at 37° C. using Methotrexate USP method with UV detection at 302 mm) of greater than 80% within 8-18 hours.

In some embodiments, the composition exhibits a substantially zero order methotrexate release rate (measured by the USP Type II dissolution apparatus (paddle method) at 100 rpm in 900 ml 0.1N hydrochloric acid at 37° C. using Methotrexate USP method with UV detection at 302 mm). In some embodiments, the composition exhibits a substantially zero order methotrexate release rate (measured by the USP Type II dissolution apparatus (paddle method) at 100 rpm in 900 ml 0.1N hydrochloric acid at 37° C. using Methotrexate USP method with UV detection at 302 mm) of greater than 80% within 8-18 hours.

In some embodiments, the compositions of the present invention are designed to provide a dissolution rate (measured by the USP Type II dissolution apparatus (paddle method) at 100 rpm in 900 ml 0.1N hydrochloric acid at 37° C.) of between 0-25% after 2 hours; between 10-60% after 4 hours; between 30-70% after 8 hours; between 20-85% after 12 hours; between 30-85% after 16 hours; and between 50-90% after 24 hours. In other embodiments, the dissolution rate (measured by the USP Type II dissolution apparatus (paddle method) at 100 rpm in 900 ml 0.1N hydrochloric acid at 37° C.) is between 0-25% after 2 hours; between 10-30% after 4 hours; between 40-80% after 8 hours; and between 50-90% after 12 hours. In still other embodiments, the dissolution rate (measured by the USP Type II dissolution apparatus (paddle method) at 100 rpm in 900 ml 0.1N hydrochloric acid at 37° C.) is between 0-20% after 6 hours; between 10-30% after 8 hours; between 20-60% after 10 hours; and between 30-85% after 12 hours.

In various embodiments, upon administration to a fed subject, the dosage form remains in the stomach for at least about 4 hours, at least about 6 hours, at least about 8 hours, or at least about 10 hours. In some embodiments, the dosage form remains in the stomach for between about 6-12 hours, between about 6-18 hours, or between about 8-10 hours. In some embodiments, upon administration of the dosage form to a fed patient, the dosage form remains in the stomach for between about 4-18 hours. In some embodiments, the dosage form remains in the stomach for between about 6-10 hours. In some embodiments, the dosage form remains in the stomach for between about 8-10 hours. In various embodiments, the dosage form remains in the stomach for at least about 8 hours. In various embodiments, the dosage form remains in the stomach for at least about 6 hours.

In some embodiments, the dosage from provides a drug Tₘₐₓ of between about 2 and about 9 hours after oral administration to a subject, or between about 3 and about 8 hours after oral administration to a subject, or between about 4 and about 7 hours after oral administration to a subject, or between about 7 and about 12 hours after oral administration to a subject, or between about 9 and about 11 hours after oral administration to a subject. In some embodiments, the drug has a Tₘₐₓ of about 5 hours after oral administration to a subject or about 7 hours after oral administration to a subject.

In some embodiments, the dosage from provides a drug Cₘₐₓ of less than about 800 nmol/L after oral administration to a subject, or about 400 nmol/L after oral administration to a subject, or below about 600 nmol/L after oral administration to a subject, or about 100 nmol/L after oral administration to a subject. In some embodiments, the drug Cₘₐₓ is between about 200 and about 800 nmol/L after oral administration to a subject, or between about 200 and about 600 nmol/L after oral administration to a subject, or between about 300 and about 500 nmol/L after oral administration to a subject, or between about 50 and about 200 nmol/L after oral administration to a subject, or between about 80 and about 120 nmol/L after oral administration to a subject.

In some embodiments, the methotrexate is present in the pharmaceutical composition in an amount of about 5 mg and wherein after oral administration of the pharmaceutical composition to a fed subject, the composition exhibits (i) a methotrexate Cₘₐₓ of between about 50 and about 250 nmol/ml, and (ii) half of the total systemic methotrexate AUC is delivered between about 4 and about 10 hours.

In some embodiments, the methotrexate is present in the pharmaceutical composition in an amount of about 10 mg and wherein after oral administration of the pharmaceutical composition to a fed subject, the composition exhibits (i) a methotrexate Cₘₐₓ of between about 100 and about 500 nmol/ml, and (ii) half of the total systemic methotrexate AUC is delivered between about 4 and about 10 hours.

In some embodiments, the methotrexate is present in the pharmaceutical composition in an amount of about 15 mg and wherein after oral administration of the pharmaceutical composition to a fed subject, the composition exhibits (i) a methotrexate Cₘₐₓ of between about 150 and about 750 nmol/ml, and (ii) half of the total systemic methotrexate AUC is delivered between about 4 and about 10 hours.

In some embodiments, the methotrexate is present in the pharmaceutical composition in an amount of about 20 mg and wherein after oral administration of the pharmaceutical composition to a fed subject, the composition exhibits (i) a methotrexate Cₘₐₓ of between about 200 and about 1000 nmol/ml, and (ii) half of the total systemic methotrexate AUC is delivered between about 4 and about 10 hours.

In some embodiments, the composition comprising methotrexate is at least about 10% more bioavailable than Trexall®, or at least about 30% more bioavailable than Trexall®, or at least about 50% more bioavailable than Trexall®. In some embodiments the group of patients consists of 6, 12, 24, 50, 100 or more than 100 patients. In some embodiments, upon administration to a patient, the methotrexate in the composition is at least about 10% more bioavailable than Trexall®. In various embodiments, the methotrexate is between about 10-50% more bioavailable than Trexall®. In some embodiments the methotrexate has a bioavailability of greater than about 40%, or greater than about 60%, or greater than about 80%. In some embodiments, the methotrexate has a bioavailability of between about 40-80%. In some embodiments, the bioavailability is determined across a group of patients. In various embodiments, the group of patients consists of 6, 12, 24, 50, 100 or more than 100 patients.

In some embodiments, upon administration to a patient, the methotrexate in the composition has a bioavailability of greater than about 40%. In some embodiments, upon administration to a patient, the methotrexate in the composition has a bioavailability of greater than about 60%. In some embodiments, upon administration to a patient, the methotrexate in the composition has a bioavailability of greater than about 70%. In some embodiments, upon administration to a patient, the methotrexate in the composition has a bioavailability of greater than about 80%. In some embodiments, upon administration to a patient, the methotrexate in the composition has a bioavailability of greater than about 90%. In some embodiments, the bioavailability is determined across a group of patients. In various embodiments, the group of patients consists of 6, 12, 24, 50, 100 or more than 100 patients.

Provided herein are pharmaceutical compositions for oral delivery comprising methotrexate and a folate which releases said methotrexate into the upper gastrointestinal tract over a sustained period of time. In some embodiments, the oral pharmaceutical composition is a monolithic solid tablet. In some embodiments, the pharmaceutical composition further comprises carbopol. In some embodiments, the methotrexate is released prior to the folate. In other embodiments, the folate is released prior to the methotrexate. In other embodiments, the folate and methotrexate are released simultaneously. In some embodiments, the folate is released from the dosage form with the methotrexate or before the methotrexate. In some embodiments, the folate is folic acid, folinic acid or 5-methyl tetrahydrofolate.

These and other features, advantages, applications and embodiments of the invention are described in more detail below.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings. The drawings included herewith are incorporated in and form a part of this specification. It should be understood that the drawings referred to in this description are not drawn to scale unless specifically noted as such.

FIG. 1 represents a diagram showing the folate metabolic pathway.

FIG. 2 represents a comparison of hypothetical plasma pharmacokinetic profiles for immediate (MTX-IR) vs. sustained release formulations containing methotrexate (MTX-SR) at 15 mg (FIG. 2A) and 5 mg (FIG. 2B) doses. Note that a Cₘₐₓ above about 400-600 nmol/L is associated with an increased risk for acute toxicities within the 24 hours following MTX oral administration. Dosing of MTX-SR results in a lower Cₘₐₓ and therefore a reduced likelihood for toxicities.

FIG. 3 represents a hypothetical plasma pharmacokinetic profile of an immediate release of methotrexate (5 mg), followed by a delayed release of 5-methyltetrahydrofolate.

FIG. 4 represents a hypothetical plasma pharmacokinetic profile of a formulation (tablet) comprising an immediate release of 5-methyltetrahydrofolate (1 mg) and a sustained release of methotrexate (5 mg).

FIG. 5 represents one shape that a dosage form of the type described herein as an equilateral triangular concave with rounded edge tooling shape, giving a slightly flattened tetrahedral shaped tablet, in accordance with one embodiment of the present invention.

FIG. 6 represents the dissolution profiles (plots of methotrexate release against time) of the formulations prepared as described in examples 1-8.

FIG. 7 represents the dissolution profiles (plots of methotrexate release against time) of the formulations prepared as described in examples 9 and 11.

FIG. 8 represents the dissolution profile (plots of methotrexate release against time) of the formulation prepared as described in example 10.

FIG. 9 represents the dissolution profiles (plots of methotrexate release against time) of the formulations prepared as described in examples 6 and 7.

FIG. 10 represents the dissolution profiles (plots of methotrexate release against time) of the formulations prepared as described in examples 2, 3, 6 and 7.

FIG. 11 represents the dissolution profiles (plots of methotrexate release against time) of the formulations prepared as described in examples 1, 4 and 5.

FIG. 12 represents the dissolution profiles (plots of methotrexate release against time) of the formulations prepared as described in examples 1 and 2.

FIG. 13 represents the dissolution profiles (plots of methotrexate release against time) of the formulations prepared as described in example 12 (FIG. 13A), example 13 (FIG. 13B) and example 14 (FIG. 13C).

FIG. 14 represents the dissolution profile (plot of methotrexate release against time) of the formulation prepared as described in example 15.

FIG. 15 represents the dissolution profile (plot of methotrexate release against time) of the formulation prepared as described in example 16.

FIG. 16 represents swelling and erosion rates (graphical plots of % swelling of the tablet matrix vs time and % erosion of the tablet matrix vs time) of the formulations prepared as described in example 12 (FIG. 16A, 16B), example 13 (FIG. 16C, 16D) and example 14 (FIG. 16E, 16F).

FIG. 17 represents a comparison of the bioadhesiveness (detachment force) of the formulations prepared as described in example examples 12, 13, 14 and Glumetza (as control) at pH 1 and pH 4.

FIG. 18 represents a comparison of the bioadhesiveness (detachment force) of the formulations prepared as described in example examples 12, 13, 14 and Glumetza (as control) at pH 1.

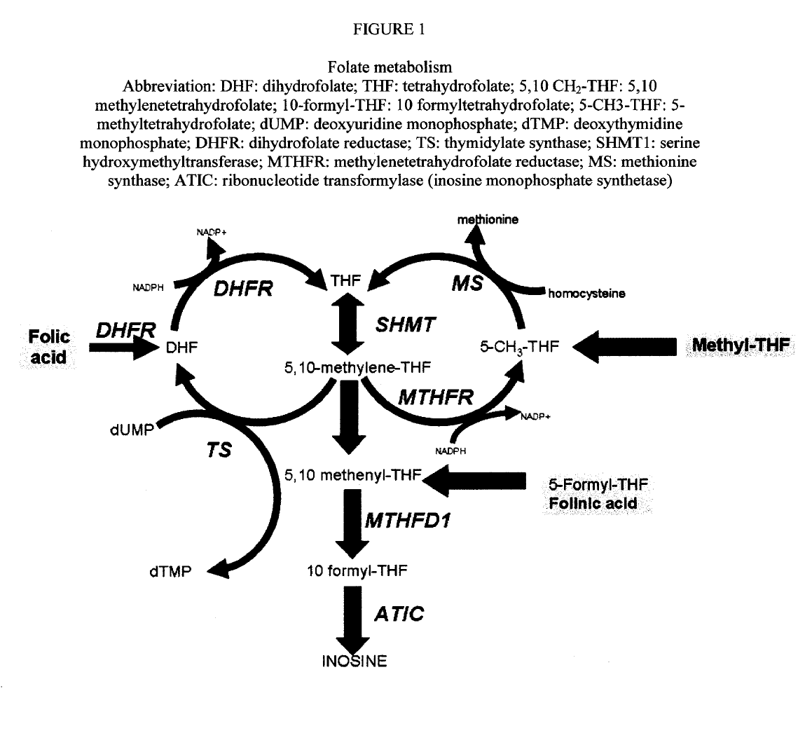

### DETAILED DESCRIPTION OF THE INVENTION

Oral administration remains the most preferred route of administration for pharmaceutical compositions. Many active agents are most effectively absorbed only from specific regions of the gastrointestinal tract, such as the stomach, the duodenum, and/or the upper portion of the small intestine. This limitation on absorption is referred to as the “absorption window” for the drug.

The Digestive Process

In the normal digestive process, the passage of matter through the stomach is delayed by a physiological condition that is commonly referred to as the digestive mode, the postprandial mode, or the “fed mode.” Between fed modes, the stomach is in the interdigestive or “fasting” mode. The difference between the two modes lies in the pattern of gastroduodenal motor activity. In the fasting mode, the stomach exhibits a cyclic activity called the interdigestive migrating motor complex (IMMC). This activity occurs in four phases:
Phase I, which lasts 45 to 60 minutes, is the most quiescent, with the stomach experiencing few or no contractions.
Phase II is characterized by sweeping contractions occurring in a irregular intermittent pattern and gradually increasing in magnitude.
Phase III consists of intense bursts of peristaltic waves in both the stomach and the small bowel. This lasts for 5 to 15 minutes.
Phase IV is a transition period of decreasing activity which lasts until the next cycle begins.

The total cycle time for all four phases is approximately 90 minutes, although it varies significantly between different individuals. The greatest activity occurs in Phase III whose powerful peristaltic waves sweep the swallowed saliva, gastric secretions, food particles, and particulate debris, out of the stomach and into the small intestine and colon. Phase III thus serves as an intestinal housekeeper, preparing the upper tract for the next meal and preventing bacterial overgrowth.

The fed mode is initiated by nutritive materials entering the stomach upon the ingestion of food. Initiation is accompanied by a rapid and profound change in the motor pattern of the upper gastrointestinal (GI) tract, over a period of 30 seconds to one minute. The change is observed almost simultaneously at all sites along the GI tract and occurs before the stomach contents have reached the distal small intestine. Once the fed mode is established, the stomach generates 3-4 continuous and regular contractions per minute, similar to those of the fasting mode but with about half the amplitude. The pylorus is partially open, causing a sieving effect in which liquids and small particles flow continuously from the stomach into the intestine while indigestible particles greater in size than the pyloric opening are retropelled and retained in the stomach. This sieving effect thus causes the stomach to retain particles exceeding about 1 cm in size for approximately 4 to 6 hours.

The particle size required for gastric retention during the fasting mode is substantially larger than the particle size required for gastric retention in the fed mode. Particles large enough to be retained in the fasting mode are too large for practical administration in most patients. Particles of a smaller particle size can be retained in the stomach if they are administered to a patient who is in the fed mode, and this offers a means of prolonging the amount of time that the particles spend in the stomach.

Described herein are sustained release pharmaceutical compositions. In some embodiments the pharmaceutical compositions comprise methotrexate. In other embodiments the pharmaceutical compositions comprise methotrexate and a folate. Also described are methods of treating diseases by administration of the pharmaceutical composition described herein. In some embodiments the disease is an autoimmune disorder. In some embodiments the disease is rheumatoid arthritis (RA), psoriasis, systemic lupus erythematosus (SLE), inflammatory bowel disease (IBD), Crohn's disease, multiple sclerosis, diabetes, graft-versus-host disease, asthma, sarcoidosis, uveitis, vasculitis or Sjogren's syndrome. In a preferred embodiment, the autoimmune disorder is rheumatoid arthritis. In other embodiments the disease is cancer. In some embodiments the disease is acute lymphocytic leukemia, meningeal leukemia, choriocarcinoma, osteosarcoma, cutaneous lymphoma, Burkitt's lymphoma, non-Hodgkin's lymphoma, breast cancer, head and neck cancer, ovarian cancer or bladder cancer.

The term “folate” as used herein refers to a member of the family of folate coenzymes, including though not limited to folic acid, folinic acid and 5-methyl tetrahydrofolate. The use of all of these folates is within the scope of the present invention.

The term “pharmaceutical composition” as used herein refers to a mixture comprising at least one drug or a derivative, pharmaceutically acceptable salt, amide, ester, prodrug or metabolite thereof, with other chemical components, such as diluents or carriers. In some embodiments the drug is methotrexate. In other embodiments the pharmaceutical composition further comprises a folate. The pharmaceutical composition facilitates administration of the drug to an organism. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, toluenesulfonic acid, salicylic acid and the like.

The term “carrier” as used herein refers to a chemical compound that facilitates the incorporation of a compound into cells or tissues. For example dimethyl sulfoxide (DMSO) is a commonly used carrier as it facilitates the uptake of many organic compounds into the cells or tissues of an organism.

The term “diluent” as used herein refers to chemical compounds diluted in water that will dissolve the compound of interest as well as stabilize the biologically active form of the compound. Salts dissolved in buffered solutions are utilized as diluents in the art. An example of a commonly used buffered solution is phosphate buffered saline because it mimics the salt conditions of human blood. Since buffer salts can control the pH of a solution at low concentrations, a buffered diluent rarely modifies the biological activity of a compound.

The term “physiologically acceptable” as used herein refers to a composition comprising at least one drug wherein any additional components of the composition do not abrogate the biological activity or properties of the drug.

The term “pharmaceutically acceptable” refers to a composition that does not cause significant irritation to an organism to which it is administered.

The term “pharmaceutically acceptable salt” as used herein refers to a formulation of a compound that does not cause significant irritation to an organism to which it is administered and does not abrogate the biological activity and properties of the compound. Pharmaceutical salts can be obtained by reacting a compound of the invention with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical salts can also be obtained by reacting a compound of the invention with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glutamine, tris(hydroxymethyl)methylamine, and salts with amino acids such as arginine, lysine, and the like.

The term “ester” as used herein refers to a chemical moiety with formula —(R)ₙ—COOR′, where R and R′ are independently selected from the group consisting of alkyl, cycloalkyl, aryl, heteroaryl (bonded through a ring carbon) and heteroalicyclic (bonded through a ring carbon), and where n is 0 or 1.

An “amide” is a chemical moiety with formula —(R)ₙ—C(O)NHR′ or —(R)ₙ—NHC(O)R′, where R and R′ are independently selected from the group consisting of alkyl, cycloalkyl, aryl, heteroaryl (bonded through a ring carbon) and heteroalicyclic (bonded through a ring carbon), and where n is 0 or 1. An amide may be an amino acid or a peptide molecule attached to a molecule of the present invention, thereby forming a prodrug.

The term “metabolite” as used herein refers to a compound to which a bisphosphonate and/or methotrexate is converted within the cells of a mammal. The pharmaceutical compositions of the present invention may include a metabolite of a bisphosphonate and/or methotrexate instead of bisphosphonate and/or methotrexate. The scope of the methods of the present invention includes those instances where a bisphosphonate and/or methotrexate is administered to the patient, yet the metabolite is the bioactive entity.

The term “prodrug” as used herein refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound of the present invention which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety.

The term “immediate release” as used herein refers to a formulation that releases its active ingredient in a single bolus or pulse.

The term “sustained release formulation” or “controlled release formulation” as used herein refers to a formulation that releases its active ingredient in a controlled fashion, for example in specified doses at timed intervals.

The term “delayed release formulation” as used herein refers to a formulation that releases its active ingredient at some point in time after administration, but not immediately. For example, oral formulations can be buffered with an enteric coating to prevent dissolution in the stomach. Instead, these formulations will release the active ingredient in the intestine.

These types of formulations (immediate release, sustained release, controlled release, delayed release) may also be combined in a single entity. For example, a tablet may be formulated with two components, one of which is released immediately, and the other which is released in a controlled manner and/or whose release is delayed. This is accomplished by manufacturing processes well known in the pharmaceutical arts in which different coatings/biopolymers are used to separate the components. These coatings differ in their chemical and physical properties, and will dissolve at different rates in different environments. Thus, the type of release and the amounts of the active ingredients released at particular times can be controlled.

Described herein are methods of treating diseases by administration of the pharmaceutical composition described herein. The terms “treating” or “treatment” do not necessarily mean total cure. Any alleviation of any undesired signs or symptoms of the disease to any extent or the slowing down of the progress of the disease can be considered treatment. Furthermore, treatment may include acts that may worsen the patient's overall feeling of well being or appearance. Treatment may also include lengthening the life of the patient, even if the symptoms are not alleviated, the disease conditions are not ameliorated, or the patient's overall feeling of well being is not improved.

Controlled Release Dosage Forms

The pharmaceutical compositions described here and useful in the present invention can provide greater therapeutic value since the active ingredient is released over a prolonged period of time, in a continuous, controlled manner. In some embodiments, the drug is released via an erosion action. The erosion can be, for example, from a controlled release of a poorly soluble drug. In other embodiments, the drug is released via a leaching action. This leaching action can be, for example, from a diffusion controlled release of highly soluble drug or a drug leaching by polymer relaxation. In some embodiments, the leaching action used to release the drug is polymer relaxation and the polymer is Carbopol.

Controlled release dosage forms function by releasing drug over an extended period of time, and thus reduce the rate of drug release to a level consistent with the blood level profile desired. Benefits of sustained release drug delivery (instead of the pulse-entry associated with conventional immediate dosage forms) include, but are not limited to:

(1) provision for more prolonged drug effects;

(2) ability to effect treatment with less frequent administration of the drug(s);

(3) reduction in quantity of drug required;

(4) reduction in side effects from the drug, and/or

(5) decreased inter and intra patient variability of the drug.

Prolonged drug effects are desirable in many therapeutic areas, such as, though not limited to prolonged control of pain or maintaining a constant level of antibiotic.

The ability to effect treatment with reduced dosing frequency, (i.e. fewer doses needed per day) would provide greater patient convenience and probably enhance patient compliance (important for drugs requiring constant dosage). Thus, for example, a continuous, controlled release dosage form could reduce administration from three or four times daily to once daily.

In addition, undesirable drug side effects may be reduced and/or eliminated by use of sustained or controlled release drug formulations. These side effects include, but are not limited to, insomnia, sedation, fatigue, malaise, stomach upset, nausea, vomiting, constipation, diarrhea, rash, headache, chills, fevers, dizziness, seizures, liver dysfunction, altered taste, myalgia, neuropathy and the like. By reducing side effects of a drug, patient compliance in taking the drug is increased. In addition, some drugs that cannot be administered to certain patient populations because of the severity of the side effects will be available for use due to the present invention.

Drugs with Small Absorption Windows

While controlled release dosage forms may deliver drugs over a prolonged period of time, this may be of little value if the dosage form spends only short periods of time in the regions of the gastrointestinal tract where the most efficient absorption occurs. The dosage form simply passes on to regions of the intestine where absorption is poor or non-existent, still releasing drug, but to no effect. Thus, administration of a drug with a small window of absorption, even from a controlled release delivery system, can still lead to sub-therapeutic blood levels and ineffective treatment of the disease state for which the drug was intended. In embodiments where the goal is to increase absorption and/or reduce dosing frequency, the release of the drug can occur in the appropriate region of the intestine, and the rate of release from the dosage form may be such as to extend and maintain effective drug plasma levels.

To achieve optimal absorption of active agents with small absorption windows, it is desirable to retain them in the stomach for as long as possible, allowing for prolonged passage from the stomach, into the duodenum and small intestine. However, many orally administered pharmaceutical compositions experience problems in retaining the dosage form in the stomach or gastrointestinal tract for sufficiently long periods of time. Indeed, active agents absorbed in the upper gastrointestinal tract with poor absorption in the distal small intestine, large intestine and colon may be regarded as unsuitable for oral formulation in many controlled delivery systems.

It is highly desirable therefore to increase the gastrointestinal residence time of these pharmaceutical compositions. Various strategies have been employed to increase the gastrointestinal residence time of pharmaceutical compositions, and are described in greater detail below. Though the goals of gastric retention and sustained release are not always compatible, dosage forms that combine these properties are desirable and are described herein.

Gastro-Retentive Systems

Described herein are dosage forms exhibiting extended gastric residence, possessing some resistance to the pattern of waves of motility present in the gastrointestinal tract that serve to propel material through it. This is achieved, in some embodiments, by simultaneously providing the dosage form with a combination of gastric residence extending characteristics, including floatation in gastric fluid, adhesion to the mucosal surfaces of the gastrointestinal tract, and swelling to a size which delays passage through the pylorus. In some embodiments, formation of microgels occurs upon exposure to gastric fluid. These properties are described in further detail below.

With the teachings described herein, those of skill in the art will be able to make and use the compositions encompassed by the methods of the present invention. In some embodiments, gastro-retentive (sustained-release) systems described herein are used in the methods of the present invention.

Floating Properties

The floating property of the dosage form is designed to have low density and thus float on gastric fluids until the dosage form either disintegrates (and the resultant particles empty from the stomach) or absorbs fluid to the point that it no longer floats and can pass more easily from the stomach with a wave of motility responsible for gastric emptying.

In some of the embodiments described herein, while the system is floating on the gastric contents, the drug is released slowly at the desired rate from the system. After release of drug, the residual system is emptied from the stomach.

In some embodiments, the system may require minimum gastric contents (at least ˜200 mL) needed to achieve proper floating principle, which can be accomplished by taking the dosage form with a cup of water. Also a minimal level of floating force (F) is required to keep the dosage form reliably buoyant on the surface of the stomach contents/meal.

Depending on the desired properties of the composition, it may be useful to use one or more of the following systems single- and multiple-unit hydrodynamically balanced systems (HBS), single and multiple-unit gas generating systems, hollow microspheres, and raft-forming systems. Various factors such as gastrointestinal physiology, dosage form characteristics, and patient-related factors will influence the dosage form buoyancy. With the knowledge in the art and the teaching provided herein, skilled artisans will readily know how to implement these systems.

In some embodiments, the floating dosage forms can be prepared where buoyancy is created via three possible mechanisms.

The first mechanism is the incorporation of formulation components with sufficiently low density to enable floating on the stomach contents. Such systems need not disintegrate into small pieces to empty from the stomach, but rather slowly erode, gradually losing buoyancy and eventually being expelled from the stomach. This approach may be especially useful for drugs administered in low doses (a few hundred milligrams per day or less) or having low water solubility. However, these properties have limited utility where higher doses are required or with highly water soluble drugs. In these instances, large amounts of polymer would be needed to retard drug release. Depending on the amount of polymer, a capsule dosage form may not be practicable due to size constraints. Furthermore, homogenous distribution of drug in a tablet of this form can be accompanied by an undesirable, rapid initial release of drug. Again, this is most often seen with very water soluble drugs.

The second mechanism is the formation of a bilayer dosage form where the buoyancy originates from a separate layer to the drug layer. This approach can overcome some of the problems encountered with the system discussed above.

The third mechanism is the incorporation of one or more gas generating agents. Gas generating agents react with gastric fluid to generate gas. This gas is subsequently entrapped within the dosage form which results in floatation in the gastric fluid. This approach may offer improved control over degree, onset time and persistence of floatation. U.S. Pat. No. 4,844,905, which is incorporated herein in its entirety, describes a system with a drug loaded core surrounded by a gas generating layer, which in turn was surrounded by a polymeric layer responsible for controlling drug release from the system. In some embodiments, the gas generating component upon interaction with gastric fluid generates carbon dioxide or sulfur dioxide that becomes entrapped within the hydrated microgel matrix of the gelling agent.

The gas generating components useful in the compositions described herein include, but are not limited to, a combination of one or more of bicarbonate and carbonate salts of Group I and Group II metals, including sodium, potassium, and calcium water soluble carbonates, sulfites and bicarbonates such as sodium carbonate, sodium bicarbonate, sodium metabisulfite, calcium carbonate. In some embodiments, the gas generating compound is sodium bicarbonate. In other embodiments, the gas generating compound is sodium carbonate. In further embodiments, the gas generating compound is calcium carbonate.

In various embodiments, the gas generating component is present in an amount from about 2-50 wt-%. In other embodiments, the gas generating compound is present in an amount of about 2-40 wt-%. In yet other embodiments, the gas generating compound is present in an amount of about 2-20 wt-%. In still other embodiments, the gas generating compound is present in an amount of about 5-10 wt-%. In yet other embodiments, the gas generating compound is present in an amount of about 1-5 wt-%. In further embodiments, the gas generating compound is present in an amount of about 1 wt-%, about 2 wt-%, about 3 wt-%, about 4 wt-%, about 5 wt-%, about 7 wt-%, about 10 wt-%, about 15 wt-%, about 20 wt-%, about 25 wt-% or about 30 wt-%.

In some embodiments, the floating tablets have a bulk density less than gastric fluid so that they remain buoyant in the stomach without affecting the gastric emptying rate for a prolonged period of time.

Limitations of floating dosage forms include required administration with a suitable amount of fluid (normal gastric contents could be as little as a few tens of milliliters) and their possible posture dependence. A patient sitting upright may ensure prolonged gastric residence of a buoyant dosage form, whereas a supine patient might allow ready presentation of the floating dosage form to the pylorus and thus allow rapid exit of the dosage form from the stomach (see Timmermans et al, J. Pharm. Sci. 1994, 83, 18-24).

Bioadhesive Properties

Bioadhesive delivery systems are designed to imbibe gastric fluid such that the outer layer becomes a viscous, tacky material that adheres to the gastric mucosa/mucus layer. This increases gastric retention until the adhesive forces are weakened for example by continuing hydration of the outer layer of the dosage form or by the persistent application of shear. Polycarbophil has been identified as a suitable polymer for adhesion of orally administered dosage forms to the gastric mucosa, (see Longer et al, J. Pharm. Sci., 1985, 74, 406-411). It should be noted that the success observed in animal models with such systems has been found to be unreliable in translating to humans due to differences in mucous amounts, consistency and turnover differences between animals and humans.

As described herein, the combination of bioadhesiveness with low density materials (i.e. less dense than gastric fluid) maintain floating while prolonging the gastric retention time (GRT) by allowing the composition to float in the upper region of the stomach. Because the dosage form also has bioadhesive characteristics, in some embodiments, the dosage form will also attach itself to gastric mucosa.

Swelling Properties

The compositions described herein should be of a size that allows the dosage form to be swallowed. After ingestion, the compositions described herein swell. In some embodiments, the compositions swell to a size that precludes passage through the pylorus until after drug release has progressed to a required degree. In some embodiments, the dosage form will swell to a size about 3 times the original size. In other embodiments, the dosage form will swell to a size about 1.5, or about 2, or about 2.5, or about 3, or about 3.5, or about 4 times the original size.

In various embodiments, the dosage form swells to its largest size within about 2 hours. In other embodiments, the dosage form swells to its largest size within about 90 minutes, or within about 60 minutes, or within about 40 minutes, or within about 30 minutes. In other embodiments, the dosage form swells to its largest size within about 20 minutes, or within about 10 minutes, or within about 5 minutes. In yet other embodiments, the dosage form swells to its largest size within about 5-90 minutes, or about 5-60 minutes, or about 2-30 minutes, or about 2-20 minutes, or about 2-10 minutes.

In some embodiments, gradual erosion of the system or its breakdown into smaller particles enables it to ultimately leave the stomach.

The dosage forms described herein can comprise hydrophilic erodible polymers. In these embodiments, upon imbibing gastric fluid the dosage form swells over a short period of time to a size that will encourage prolonged gastric retention. This allows for the sustained delivery of the drug to the absorption site. In some embodiments, the absorption site of the drug is in the upper gastrointestinal tract.

When the dosage forms are made of an erodible, hydrophilic polymer(s), they readily erode over a reasonable time period to allow passage from the stomach. The time period of expansion is such that this will not occur in the esophagus and if the dosage form passes into the intestine in a partially swollen state, the erodibility and elastic nature of the hydrated polymer will eliminate the chance of intestinal obstruction by the dosage form.

Various types of polymers are available to provide systems that will swell and then gradually release drug from the swollen dosage forms. For example, drug dissolution dosage forms can comprise linear hydrophilic polymers. Upon hydration, these linear hydrophilic polymers, which do not have a covalently cross-linked structure, can form a gelatinous layer on the surface of the dosage form. The thickness and durability of this gelatinous layer depends on a number of factors such as the concentration, molecular weight and viscosity of the polymer(s) comprising the dosage form. At higher concentrations the linear polymer chains entangle to a greater degree. This can result in virtual cross-linking and the formation of a stronger gel layer. As the swollen linear chains of the hydrophilic polymer dissolve, the gel layer erodes and the drug is released. In these embodiments, the rate of dosage form erosion helps control the release rate of the drug.

In further embodiments, cross-linked polymers such as polyacrylic acid polymer (PAA) may be used in the dosage form matrix. In the dry state, dosage forms formulated with cross-linked polyacrylic acid polymers contain the drug trapped within a glassy core. In these embodiments of the present invention, as the external surface of the tablet is hydrated, it forms a gelatinous layer. It is believed that this layer is different than traditional matrices because the hydrogels are not entangled chains of polymer, but discrete microgels made up of many polymer particles. The crosslink network enables the entrapment of drugs in the hydrogel domains. Because these hydrogels are not water soluble, they do not dissolve or erode in the same manner as linear polymers. Instead, when the hydrogel is fully hydrated, osmotic pressure from within works to break up the structure by sloughing off discrete pieces of the hydrogel. The drug is able to diffuse through the gel layer at a uniform rate.

Though not wishing to be bound by any particular theory, it is postulated that as the concentration of the drug increases within the gel matrix and its thermodynamic activity or chemical potential increases, the gel layer around the drug core acts as a rate controlling membrane, which results in a linear release of the drug. With these systems, drug dissolution rates are affected by subtle differences in rates of hydration and swelling of the individual polymer hydrogels. These properties of the polymer hydrogels are dependent on various factors such as the molecular structure of the polymers, including crosslink density, chain entanglement, and crystallinity of the polymer matrix. The extent and rate of swelling is also dependent on pH and the dissolution medium. The channels that form between the polymer hydrogels are also influenced by the concentration of the polymer and the degree of swelling. Increasing the amount of polymer or the swelling degree of the polymer decreases the size of the channels.

Cross-linked polyacrylic acid polymers provide rapid and efficient swelling characteristics in both simulated gastric fluid (SGF) and simulated intestinal fluid (SIF) and produce dosage forms of excellent hardness and low friability. Moreover, cross-linked polyacrylic acid polymers may also provide longer dissolution times at lower concentrations than other excipients.

Drug solubility is also important to drug release from dosage forms comprising cross-linked polyacrylic acid polymers. Poorly soluble drugs tend to partition into the more hydrophobic domains of the system, such as the acrylic backbone of the polymer. Highly water soluble drugs undergo diffusion controlled-release due to the fast dissolution of the drug through the water-filled interstitial spaces between the microgels. In some embodiments, the use of lightly cross-linked polyacrylic acid polymers results in the drug being more likely to partition in the hydrophilic matrix of the polymer and exhibit nonlinear diffusion.

With the combination of sufficient swelling, floatation and/or bioadhesion properties, the dosage forms described and useful in the present invention achieve gastric retention regardless of whether the subject is in the fed mode or the fasting mode.

One means of achieving a swellable particle is to disperse the drug in a solid matrix formed of a substance that absorbs the gastric fluid and swells as a result of the absorbed fluid. See., e.g., U.S. Pat. Nos. 5,007,790, 5,582,837 and 5,972,389, and WO 98/55107, each of which are incorporated by reference herein in their entirety.

Polymer matrices are useful for achieving controlled release of the drug over a prolonged period of time. Such sustained or controlled release is achieved either by limiting the rate by which the surrounding gastric fluid can diffuse through the matrix and reach the drug, dissolve the drug and diffuse out again with the dissolved drug, or by using a matrix that slowly erodes. See, e.g., U.S. Pat. Nos. 4,915,952, 5,328,942, 5,451,409, 5,783,212, 5,945,125, 6,090,411, 6,120,803, 6,210,710, 6,217,903, and WO 96/26718 and WO 97/18814), each of which are incorporated by reference herein in their entirety.

U.S. Pat. No. 4,434,153, which is incorporated by reference in its entirety, describes the use of a hydrogel matrix that imbibes fluid to swell to reach a size encouraging prolonged gastric retention. This matrix surrounds a plurality of tiny pills consisting of drug with a release rate controlling wall of fatty acid and wax surrounding each of the pills.

U.S. Pat. Nos. 5,007,790 and 5,582,837, and WO 93/18755, each of which are incorporated by reference herein in their entirety, describe a swelling hydrogel polymer with drug particles embedded within it. These particles dissolve once the hydrogel matrix is hydrated. The swollen matrix is of a size to encourage gastric retention but only dissolved drug reaches the mucosa and this can be delivered in a sustained manner. Such a system thus does not insult the mucosa with solid particles of irritant drug and is suitable for delivering drug to the upper gastrointestinal tract. These systems only apply in case of drugs of limited water solubility.

Layered Gastroretentive Systems

The layered gastroretentive drug delivery systems described in U.S. Pat. No. 6,685,962, and incorporated herein in its entirety, can be used in the sustained release delivery methods described herein. In general, such delivery systems have an active agent or drug associated with a matrix that is affixed or attached to a membrane. The membrane prevents evacuation from the stomach thereby allowing the active agent/matrix to be retained in the stomach for 3-24 hours.

The matrix/membrane system can be a multilayer system, including but not limited to a bilayer system. In addition, the matrix/membrane may be administered as a folded configuration within a capsule, including but not limited to a gelatin capsule.

The matrix of such delivery systems can be a single- or multi-layered and have a two- or three-dimensional geometric configuration. The matrix can comprise a polymer selected from a degradable polymer, including but not limited to a hydrophilic polymer which is not instantly soluble in gastric fluids, an enteric polymer substantially insoluble at pH less than 5.5, a hydrophobic polymer; or any mixture thereof. In addition, the matrix can comprise a non-degradable; or a mixture of at least one degradable polymer and at least one non-degradable polymer.

The hydrophilic polymers of such delivery systems may be any hydrophilic polymer, including but not limited to, a protein, a polysaccharide, a polyacrylate, a hydrogel or any derivative thereof. By way of example only, such proteins are proteins derived from connective tissues, such as gelatin and collagen, or an albumin such as serum albumin, milk albumin or soy albumin. By way of example only, such polysaccharides are sodium alginate or carboxymethylcellulose. By way of example only, other hydrophilic polymers may be polyvinyl alcohol, polyvinyl pyrrolidone or polyacrylates, such as polyhydroxyethylmethacrylate. In addition, the hydrophilic polymer may be cross-linked with a suitable cross-linking agent. Such cross-linking agents are well known in the art, and include, but are not limited to, aldehydes (e.g. formaldehyde and glutaraldehyde), alcohols, di-, tri- or tetravalent ions (e.g. aluminum, chromium, titanium or zirconium ions), acyl chlorides (e.g. sebacoyl chloride, tetraphthaloyl chloride) or any other suitable cross-linking agent, such as urea, bis-diazobenzidine, phenol-2,4-disulfonyl chloride, 1,5-difluoro-2,4-dinitrobenzene, 3,6-bis-(mercuromethyl)-dioxane urea, dimethyl adipimidate, N,N′-ethylene-bis-(iodoacetamide) or N-acetyl homocysteine thiolactone. Other suitable hydrogels and their suitable cross-linking agents are listed, for example, in the Handbook of Biodegradable Polymers [A. J. Domb, J. Kost & D. M. Weisman, Eds. (1997) Harwood Academic Publishers], incorporated herein by reference.

The enteric polymer used in such layered delivery systems is a polymer that is substantially insoluble in a pH of less than 5.5. By way of example only, such enteric polymers include shellac, cellulose acetate phthalate, hydroxypropyl methylcellulose phthalate, hydroxypropyl methylcellulose acetate succinate or methylmethacrylate-methacrylic acid copolymers.

The non-degradable hydrophobic polymers used in such layered delivery systems include, but are not limited to, ethylcellulose, acrylic acid-methacrylic acid esters copolymer, polyethylene, polyamide, polyvinylchloride, polyvinyl acetate and mixtures thereof.

The degradable hydrophobic polymers used in such layered delivery systems include, but are not limited to, poly(.alpha.-hydroxyacids), such as poly(lactic acid), poly(glycolic acid), copolymers and mixtures thereof.

The membranes used in such layered delivery systems have substantial mechanical strength and may be continuous or non-continuous. Such membranes may comprise, by way of example only, cellulose ethers and other cellulose derivatives such as cellulose nitrate, cellulose acetate, cellulose acetate butyrate or cellulose acetate propionate; polyesters, such as polyethylene terephthalate, polystyrene, including copolymers and blends of the same; polylactides, including copolymers thereof with p-dioxanone, polyglycolides, polylactidglycolides; polyolefins, including polyethylene, and polypropylene; fluoroplastics, such as polyvinylidene fluoride and polytetrafluoroethylene, including copolymers of the same with hexafluoropropylene or ethylene; polyvinylchloride, polyvinylidene chloride copolymers, ethylene vinyl alcohol copolymers, polyvinyl alcohols, ammonium-methacrylate copolymers and other polyacrylates and polymethacrylates; polyacrylonitriles; polyurethanes; polyphthalamides; polyamides; polyimides; polyamide-imides; polysulfones; polyether sulfones; polyethylene sulfides; polybutadiene; polymethyl pentene; polyphenylene oxide (which may be modified); polyetherimides; polyhydroxyalkanoates; tyrosine derived polyarylates and polycarbonates including polyester carbonates, polyanhydrides, polyphenylene ethers, polyalkenamers, acetal polymers, polyallyls, phenolic polymers, polymelamine formaldehydes, epoxy polymers, polyketones, polyvinyl acetates and polyvinyl carbazoles.

The active agent or drug associated with the matrix may be in a particulate form or may be in the form of raw powder, or soluted, dispersed or embedded in a suitable liquid, semisolid, micro- or nanoparticles, micro- or nanospheres, tablet, or capsule. The drug, or mixtures of drugs, in any of such forms, may be embedded in at least one layer of the matrix of the delivery system. Alternatively, in a multi-layered matrix, including but not limited to a bi-layered matrix, the drug may be entrapped between any two layers, whether in free form or contained within a drug-containing means such as, by way of example only, in a tablet or a capsule.

Microcapsule Gastroretentive Systems

The microcapsules gastroretentive systems described in U.S. Pat. Nos. 6,022,562, 5,846,566 and 5,603,957, each herein incorporated by reference in their entirety, can be used in the sustained release delivery methods described herein. Microparticles of an active agent or drug are coated by spraying with a material consisting of a mixture of a film-forming polymer derivative, a hydrophobic plasticizer, a functional agent and a nitrogen-containing polymer. The resulting microcapsules are less than or equal to 1000 microns (μm) in size, and in certain cases such microcapsules are between 100 and 500 microns. These microcapsules remain in the small intestine for at least 5 hours.

Film-forming polymer derivatives used in such microcapsules include, but are not limited to, ethylcellulose, cellulose acetate, and non-hydrosoluble cellulose derivates. The nitrogen-containing polymers include, but are not limited to, polyacrylamide, poly-N-vinylamide, poly-N-vinyl-lactam and polyvinylpyrrolidone. The plasticizer used in such microcapsule include, but are not limited to, glycerol esters, phthalates, citrates, sebacates, cetylalcohol esters, castor oil and cutin. The surface-active and/or lubricating agent used in such microcapsule include, but are not limited to, anionic surfactants, such as by way of example the alkali metal or alkaline-earth metal salts of fatty acids, stearic acid and/or oleic acid, nonionic surfactants, such as by way of example, polyoxyethylenated esters of sorbitan and/or polyoxyethylenated esters of sorbitan and/or polyoxyethylenated derivatives of castor oil; and/or lubricants such as stearates, such as by way of example, calcium, magnesium, aluminium stearate, zinc stearate, stearylfumarate, sodium stearylfimarate, and glyceryl behenate.

Characteristics of the Formulation

The drug/polymer mixture is in the form of a plurality of particles. In some embodiments, the solid drug is dispersed homogeneously throughout the polymer. In further or additional embodiments the solid drug is dispersed non-homogeneously throughout the polymer. As one non limiting example, the non-homogenous distribution creates a pulsed release, even more closely mirroring a split dose of drug.

In some embodiments, upon administration to a subject the composition provides delivery of a drug such that a therapeutic effect is achieved for at least about 24 hours. In other embodiments, upon administration to a subject the composition provides delivery of a drug such that a therapeutic effect is achieved for at least about 18 hours. In yet other embodiments, upon administration to a subject the composition provides delivery of a drug such that a therapeutic effect is achieved for at least about 12 hours or at least about 8 hours.

In some embodiments, upon administration to a subject the composition provides delivery of a drug with improved pharmacokinetic and/or pharmacodynamic properties. In some embodiments, upon administration to a subject the composition provides delivery of a drug with decreased toxicity. In some embodiments, upon administration to a subject the composition provides delivery of a drug with decreased side effects. In some embodiments, upon administration to a subject the composition provides delivery of a drug over a sustained period of time.

Amount of Drug

Pharmaceutical compositions suitable for use in the present invention include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

Note that for many drugs, human dosages for treatment of at least some condition have been established. Thus, in most instances, the present invention will use those same dosages, or dosages that are between about 0.1% and 500%, more preferably between about 25% and 250% of the established human dosage. Where no human dosage is established, as will be the case for newly-discovered pharmaceutical compounds, a suitable human dosage can be inferred from ED₅₀ or ID₅₀ values, or other appropriate values derived from in vitro or in vivo studies, as qualified by toxicity studies and efficacy studies in animals.

The exact formulation, route of administration and dosage for the pharmaceutical compositions can be chosen by the individual physician in view of the patient's condition. (See e.g., Fingl et al. 1975, in “The Pharmacological Basis of Therapeutics”, Ch. 1 p. 1). Typically, the dose range of the composition administered to the patient can be from about 0.5 to 1000 mg/kg of the patients body weight. The dosage of each component may be a single one or a series of two or more given in the course of one or more days, as is needed by the patient

Although the exact dosage will be determined on a drug-by-drug basis, in most cases, some generalizations regarding the dosage can be made. The daily dosage regimen for an adult human patient may be, for example, an oral dose of between 0.1 mg and 6000 mg of each ingredient, preferably between 1 mg and 5000 mg, e.g. 25 to 5000 mg of each ingredient of the pharmaceutical compositions of the present invention or a pharmaceutically acceptable salt thereof calculated as the free base, the composition being administered 1 to 4 times per day. Thus, the total daily dosage by oral administration of each ingredient will typically be in the range 1 to 2500 mg and the total daily dosage by parenteral administration will typically be in the range 0.1 to 400 mg. Suitably the compounds will be administered for a period of continuous therapy, for example for a week or more, or for months or years.

In one embodiment, the oral pharmaceutical compositions comprise between about 2.5 and 40 mg of methotrexate. In another embodiment, the oral pharmaceutical compositions comprise between about 2.5 and 40 mg of methotrexate, and between about 0.5 and 8 mg of folate. In another embodiment, enough of the composition is administered to provide between about 25 and 400 mg methotrexate. In another embodiment, enough of the composition is administered to provide between about 25 and 400 mg methotrexate, and between about 5 and 80 mg of folate. In another embodiment, these compositions are administered once a week.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen that maintains drug plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The pharmaceutical compositions described herein are for oral administration. In some embodiments administration is to a human patient. In some embodiments the administration is to a human patient per se, while in other embodiments the pharmaceutical compositions are mixed with other active ingredients, as in combination therapy, or with additional suitable carriers or excipient(s). Techniques for formulation and administration of the compounds of the instant application may be found in “Remington's Pharmaceutical Sciences,” Mack Publishing Co., Easton, Pa., 18th edition, 1990

Ratios of Drug/Polymer

The weight ratio of the polymer to the dosage form weight in the mixture or dispersion will normally be between about 1:50 to about 3:1, preferably about 1:9 to 2:1, and most preferably about 1:7 to 1:1.

In some embodiments, the weight percent of the polymer in the dosage form is about 10-75 wt-% of dosage form. In other embodiments, the weight percent of the polymer in the dosage from is about 12-50 wt-% of dosage form. In still other embodiments, the weight percent of the polymer in the dosage form is about 20-40 wt-% in the dosage form weight. In still other embodiments, the weight percent of the polymer in the dosage form is about 20-30 wt-% in the dosage form weight.

Dosage Form Shape

Tetrahedral shaped dosage forms can have up to 2 times longer gastric retention time (GRT) than either round or oblong shaped tablets. In some embodiments, the dosage forms described herein may be diamond shaped with a triangular lateral length of about 6-15 mm with vertical axis length of about 2-12 mm. In some embodiments the vertical axis length is about 6 mm, about 7 mm, about 8 mm, about 9 mm, about 10 mm, about 11 mm, about 12 mm, about 13 mm, about 14 mm or about 15 mm. In some embodiments the vertical axis length is at least 6 mm, at least 7 mm, at least 8 mm, at least 9 mm, at least 10 mm, at least 11 mm, at least 12 mm, at least 13 mm, at least 14 mm or at least 15 mm. In some embodiments the lateral length is about 2 mm, about 3 mm, about 4 mm, about 5 mm, about 6 mm, about 7 mm, about 8 mm, about 9 mm, about 10 mm, about 1 mm or about 12 mm. In some embodiments the lateral length is at least 2 mm, is at least 3 mm, is at least 4 mm, is at least 5 mm, is at least 6 mm, is at least 7 mm, is at least 8 mm, is at least 9 mm, is at least 10 mm, is at least 11 mm or at least 12 mm. In some preferred embodiments the vertical axis length is about 8-14 mm and the lateral length is about 5-9 mm. See AAPS PharmSci Tech. 2005; 6(3) E372-390, which is incorporated by reference herein in its entirety.

In other embodiments, the dosage may be a trigonal bipyramid, triangular with rounded corners, tetrahedral or annular in shape, though other shapes may also be used.

Dosage Form Size

The initial pre-ingestion (non-swollen) dosage form size is in the range of about 2 to 14 mm, as measured along the largest dimension of the particle. In some embodiments, the largest dimension is about 5-14 mm. In other embodiments, the largest dimension is about 7-14 mm. In other embodiments, the largest dimension is about 9-14 mm. The thickness of the initial pre-ingestion (non-swollen) dosage form is in the range of about 5 to 8 mm. In some embodiments, the thickness is about 6-7 mm. In some embodiments, the dosage form is a triangular shape with a size of about 7-14 mm on all sides. In some embodiments the dosage form is a triangular shape and each side has a dimension of about 9-14 mm. In other embodiments each side has a dimension of about 12 mm.

The swollen dosage form will be of a size that promotes their retention in the stomach when the patient is in the fed mode (i.e., in the presence of food) and or the unfed or fasted mode.

In some embodiments, the swollen dosage form may be of a size in the range of about 10 to 28 mm, preferably about 15 to about 25 mm, as measured along the largest dimension of the particle, though may be larger.

Alternatively, the particles may swell to twice their original size, three times their original size or four times their original size, as measured along the largest dimension of the particle.

The dosage form will absorb gastric fluid upon ingesting and swell to a size that is large enough to be retained in the stomach for a minimum of 4-8 hours. In some embodiments, the gastric retention of the dosage form is for a minimum of about 5-8 hours. In other embodiments, the gastric retention of the dosage form is for a minimum of 6-8 hours. In yet other embodiments, the gastric retention of the dosage form is for a minimum of 8-9 hours. In still other embodiments, the gastric retention of the dosage form is for a minimum of 6-12 hours. In further embodiments, the gastric retention of the dosage form is for a minimum of 16 hours.

Swelling Rate

The particles may swell up to their largest size, as measured along the largest dimension of the particle, in about 5 minutes to about 8 hours. In some embodiments, the particles swell to their largest size in about 30 minutes. In other embodiments, the particles swell to their largest size in about 45 minutes, or about 1 hour, or about 1.5 hours, or about 2 hours, or about 2.5 hours, or about 3 hours, or about 3.5 hours, or about 4 hours. In other embodiments, the particles swell to their largest size in about 5 minutes to about 3 hours, or about 30 minutes to about 4 hours. In various embodiments, the particles swell to their largest size in about 1-2.5 hours. In other embodiments, the particles swell to their largest size in about 1 hour.

In some embodiments, the particles may swell up to two times their original size, as measured along the largest dimension of the particle, in about 30 minutes to about 8 hours. In other embodiments, the particles may swell up to three times their original size, as measured along the largest dimension of the particle, in about 30 minutes to about 8 hours. In still other embodiments, the particles may swell up to four times their original size, as measured along the largest dimension of the particle, in about 30 minutes to about 8 hours.

Erosion Rate

The swollen volume of the composition will decrease at a substantially constant rate over the dosing period. The swollen volume of the composition will decrease slowly over the dosing period. Degree of agitation and existence of external matters such as food particles impact the erosion rate.

In some embodiments 10-30% of the composition erodes within 1 hour after immersion in gastric fluid. In some embodiments 15-20% of the composition erodes within 1 hour after immersion in gastric fluid. In some embodiments 20-50% of the composition erodes within 9 hours after immersion in gastric fluid.

Drug Release Rate

Upon immersion in gastric fluid, the composition will begin to release said drug. Upon immersion in gastric fluid, the composition releases said drug at a substantially constant rate over the dosing period. The composition release rate will decrease slowly over the dosing period. Degree of agitation and existence of external matters such as food particles impact the release rate.

In some embodiments, upon immersion in gastric fluid, the composition releases about 50% of said drug between about 4 and about 10 hours. In other embodiments, upon immersion in gastric fluid, the composition releases about 75% of said drug between about 3 and about 12 hours. In yet other embodiments, upon immersion in gastric fluid, said composition retains at least about 50% of said drug 7 hours after immersion; releases 75% of said drug within about 15 hours after immersion, and releases substantially all of said drug within about 16 hours after immersion.

Pharmacokinetic Properties

Increased Gastrointestinal Residence Time

The dosage forms of the present invention are designed to be maintained in the stomach for a period of time which allows the drug to be administered effectively. In some embodiments, the dosage form remains in the stomach for at least about 4 hours, at least about 6 hours, at least about 8 hours or at least about 10 hours. Some of the dosage forms described herein remain in the stomach for between about 6-12 hours, or between about 12-18 hours, or between about 8-10 hours.

Increased Bioavailability

The dosage forms of the present invention can be used to increase the bioavailability of certain drugs that are transported by one or more active transporters, and such transporter has saturation concentration. The bioavailability of drugs with a concentration dependent active transporter can have a lower bioavailability as the dosage strength increases.

For example, the bioavailability of orally dosed methotrexate is between about 30-40% in higher dose strength (10-15 mg/dose) while at lower dose strength bioavailability of orally administered methotrexate can be as high as 60%. This is believed to be due, in part, to the presence of an absorption window restricted to the upper gastrointestinal tract which can be easily saturated after a threshold concentration. Methotrexate also has side effects associated with high Cₘₐₓ concentrations of the drug. As such, methotrexate is a good candidate for demonstrating the effectiveness of the dosage forms described herein. For example, the dosage forms can be used to increase the bioavailability of the drug methotrexate by avoiding saturation of absorption processes evident in some patients at higher weekly dosage (˜15-25 mg/week). For example, one hypothesis is that the transporter for methotrexate (PCFT/HCP1) becomes saturated. In these embodiments, the gastro-retentive delivery system can improve bioavailability by allowing the transporter to work over a longer period of time. In some of these embodiments, the methotrexate in the controlled-release formulation is at least about 10% more bioavailable than Trexall® or Rheumatrex, or at least about 20% more bioavailable than Trexall® or Rheumatrex, or at least about 30% more bioavailable than Trexall® or Rheumatrex, or at least about 40% more bioavailable than Trexall® or Rheumatrex, or at least about 50% more bioavailable than Trexall® or Rheumatrex. The increased bioavailability of the drug can be in a single patient or the average of a group of patients (i.e., anywhere between 5-1000 patients). In other embodiments, the methotrexate has a bioavailability of greater than about 40%, or greater than about 50%, or greater than about 60%, or greater than about 70%, or greater than about 80%. Alternatively, the methotrexate in controlled-release formulations may have a bioavailability of between about 40-90%, or 40-80%, or 40-50%, or 50-60%, or 60-70%, or 70-80%, or 80-90%. In some embodiments, the bioavailability is determined from the average of a group of patients (i.e., anywhere between 5-1000 patients). In various embodiments the group of patients is 6, 12, 24, 50, 100 or greater than 100 patients.

With the teachings herein, the skilled artisan will recognize that similar increases in bioavailability can be achieved for other drugs when the methods described herein are utilized.

Maximum Plasma Concentration (Cₘₐₓ) and Time to Maximum Plasma Concentration (Tₘₐₓ)

The dosage forms of the present invention are sustained release formulations. In some embodiments, the Tₘₐₓ of the active ingredient is between about 2-9 hours after oral administration of the composition to the subject. In other embodiments, the Tₘₐₓ of the active ingredient is between about 3-8 hours, or about 4-7 hours, or about 7-12 hours, or about 9-11 hours. In some embodiments, the Tₘₐₓ of the active ingredient is about 6 hours. In other embodiments, the Tₘₐₓ of the active ingredient is about 10 hours.

The dosage forms of the present invention may also have different Cₘₐₓ values, depending on the drug being administered. For illustrative purposes only, the Cₘₐₓ of the active ingredient may be less than about 800 nmol/L or less than about 1000 nmol/L after administration. The Cₘₐₓ of the active ingredient can be between about 50 and about 200 nmol/L or between about 80 and about 120 nmol/L or between about 200 and about 800 mmol/L. or between about 200 and about 600 nmol/L, or between about 300 and about 500 nmol/L after administration. In some embodiments, the drug has a Cₘₐₓ of about 100 nmol/L, or about 200 nmol/L, or about 300 nmol/L, or about 400 nmol/L or about 500 nmol/L, or about 600 nmol/L or about 700 nmol/L, or about 800 nmol/L, or about 900 nmol/L, or about 1000 nmol/L. In some embodiments, the active ingredient being administered is methotrexate.

In some embodiments, the drug is methotrexate and, after oral administration of the composition, the Cₘₐₓ is between about 200 and about 800 nmol/L and the Tₘₐₓ is between about 1 and about 9 hours. In other embodiments, the drug is methotrexate and, after oral administration of the composition, the Cₘₐₓ is between about 300 and about 500 nmol/L and the Tₘₐₓ is between about 4 and about 7 hours. In various embodiments, the amount of drug administered is between about 1-30 mg per tablet.

In some embodiments, the Cₘₐₓ of the active ingredient in the composition of the present invention is at least about 50% lower that the Cₘₐₓ of an immediate-release formulation containing the same amount of active ingredient. In various embodiments, the Cₘₐₓ of the active ingredient in the composition of the present invention is at least about 30% lower that the Cₘₐₓ of an immediate-release formulation containing the same amount of active ingredient.

In other embodiments, the Cₘₐₓ of the active ingredient in the composition of the present invention is between about 20%-70% lower that the Cₘₐₓ of an immediate-release formulation containing the same amount of active ingredient. In yet other embodiments, the Cₘₐₓ of the active ingredient in the composition of the present invention is between about 30%-50% lower that the Cₘₐₓ of an immediate-release formulation containing the same amount of active ingredient.

In some embodiments, the methotrexate is present in the invention in an amount of about 5 mg and, upon administration to a subject, a methotrexate Cₘₐₓ of between about 50 and about 250 nmol/ml is observed. In some embodiments, the methotrexate is present in the invention in an amount of about 10 mg and, upon administration to a subject, a methotrexate Cₘₐₓ of between about 100 and about 500 nmol/ml is observed. In some embodiments, the methotrexate is present in the invention in an amount of about 15 mg and, upon administration to a subject, a methotrexate Cₘₐₓ of between about 150 and about 750 nmol/ml is observed. In some embodiments, the methotrexate is present in the invention in an amount of about 20 mg and, upon administration to a subject, a methotrexate Cₘₐₓ of between about 200 and about 1000 nmol/ml is observed.

In some embodiments, half of the total systemic methotrexate AUC is delivered between about 4 and about 16 hours. In some embodiments, half of the total systemic methotrexate AUC is delivered between about 4 and about 12 hours. In some embodiments, half of the total systemic methotrexate AUC is delivered between about 4 and about 8 hours. In some embodiments, half of the total systemic methotrexate AUC is delivered between about 6 and about 10 hours.

In Vitro Dissolution

The compositions described herein are formulated to have particular in vitro dissolution properties. In some embodiments, the composition exhibits a dissolution rate (measured by the USP Type II dissolution apparatus (paddle method) at 100 rpm in 900 ml 0.1N hydrochloric acid at 37° C.) of between 0-25% after 2 hours; between 10-60% after 4 hours; between 30-70% after 8 hours; between 20-85% after 12 hours; between 30-85% after 16 hours; and between 50-90% after 24 hours. In alternative embodiments, the composition exhibits a dissolution rate (measured by the USP Type II dissolution apparatus (paddle method) at 100 rpm in 900 ml 0.1N hydrochloric acid at 37° C.) of between 0-25% after 2 hours; between 10-30% after 4 hours; between 40-80% after 8 hours; and between 50-90% after 12 hours. In still other embodiments, the composition exhibits a dissolution rate (measured by the USP Type II dissolution apparatus (paddle method) at 100 rpm in 900 ml 0.1N hydrochloric acid at 37° C.) of between 0-20% after 6 hours; between 10-30% after 8 hours; between 20-60% after 10 hours; and between 30-85% after 12 hours.

When the composition contains methotrexate, the dissolution rate is measured by the USP Type II dissolution apparatus (paddle method) at 100 rpm in 900 ml 0.1N hydrochloric acid at 37° C. using methotrexate USP method with UV detection at 302 mm.

Components of the Formulation

Active Agents (Drugs)

Drugs of relatively high solubility are generally considered to be those whose solubility in water at 37° C. is greater than one part by weight of the drug in twenty parts by weight of water.

Examples of drugs of high solubility are metformin hydrochloride, gabapentin, losartan potassium, vancomycin hydrochloride, captopril, erythromycin lactobionate, ranitidine hydrochloride, sertraline hydrochloride, ticlopidine hydrochloride, tramadol, fluoxetine hydrochloride, bupropion, lisinopril, iron salts, sodium valproate, valproic acid, and esters of ampicillin. Additional examples of highly soluble drugs are abacavir, acetaminophen, Acyclovir, amiloride, amitryptyline, antipyrine, atropine, buspirone, caffeine, captopril, chloroquine, chlorpheniramine, cyclophosphamide, desipramine, diazepam, diltiazem, diphenhydramine, disopyramide, doxepin, doxycycline, enalapril, ephedrine, ergonavine, eronovine, ethambutol, ethinyl estradiol, fluoxetine, glucose, imipramine, ketorolac, ketoprofen, labetolol, levodopa, levofloxacin, lidocaine, lomefloxacin, meperidine, metoprolol, metronidazole, midazolam, minocycline, misoprostol, nifedipine, phenobarbital, phenylalanine, prednisolone, primaquine, promazine, propranolol, quinidine, rosiglitazone, salicylic acid, theophylline valproic acid verapamil, zidovudine, acyclovir, amiloride, amoxicillin, atenolol, atropine, bisphosphonates, bidisomide, captopril, cefazolin, cetirizine, cimetidine, ciprofloxacin, cloxacillin, dicloxacillin, erythromycin, famotidine, fexofenadine, folinic acid, furosemide, ganciclovir, hydrochlorothiazide, lisinopril, metformin, methotrexate, nadolol, pravastatin, penicillins, ranitidine, tetracycline, trimethoprim, valsartan, and zalcitabine.

Drugs of relatively low solubility are generally considered to be those whose solubility in water at 37° C. is from about 0.005% to about 5% by weight. Examples of drugs of low solubility are cefaclor, ciprofloxacin (and its hydrochloride salt), saguinavir, ritonavir, nelfinavir, clarithromycin, azithromycin, ceftazidine, cyclosporin, digoxin, paclitaxel, methotrexate, dichlorofenac (and its salt), theopholline, and ketoconazole. Additional examples of drugs with low solubility are amiodarone, atorvastatin, azithromycin, carbamazepine, carvedilol, chlorpromazine, cisapride, ciprofloxacin, cyclosporine, danazol, dapsone, diclofenac, diflunisal, digoxin, erythromycin, flurbiprofen, glipizide, glyburide, griseofulvin, ibuprofen, indinavir, indomethacin Itraconazole, ketoconazole, lansoprazole, lovastatin, mebendazole, naproxen, nelfinavir, ofloxacin, oxaprozin, phenazopyridine, phenyloin, piroxicam, raloxifene, ritonavir, saquinavir, sirolimus, spironolactone, tacrolimous, talinolol, tamoxifen, terfenadine, warfarin ampheotericin B, chlorthalidone, chlorothiazide, colistin, ciprofloxacin, furosemide, hydrochlorothiazide, mebendazole, methotrexate, and neomycin.

The amount of drug included in the compositions can vary, but is generally between about 1-1000 mgs. As is known in the art, the dosage amounts can vary based on a variety of factors such as the drug being administered, the size of the patient and the delivery system being used. The amount of drug useful in the compositions of the present invention can be readily determined by those of skill in the art.

It should be noted that some of the ranges cited above for high solubility drugs overlap or are contiguous (share a limit) with the some of the ranges for low solubility drugs. While “high solubility” and “low solubility” are terms whose meaning will be understood by those skilled in the art of pharmaceutical drugs, the terms are relative by nature, and the overlapping portions refer to drugs that are intermediate in solubility, i.e., “high solubility” drugs that are close to the “low solubility” range and vice versa.

Methotrexate Therapy

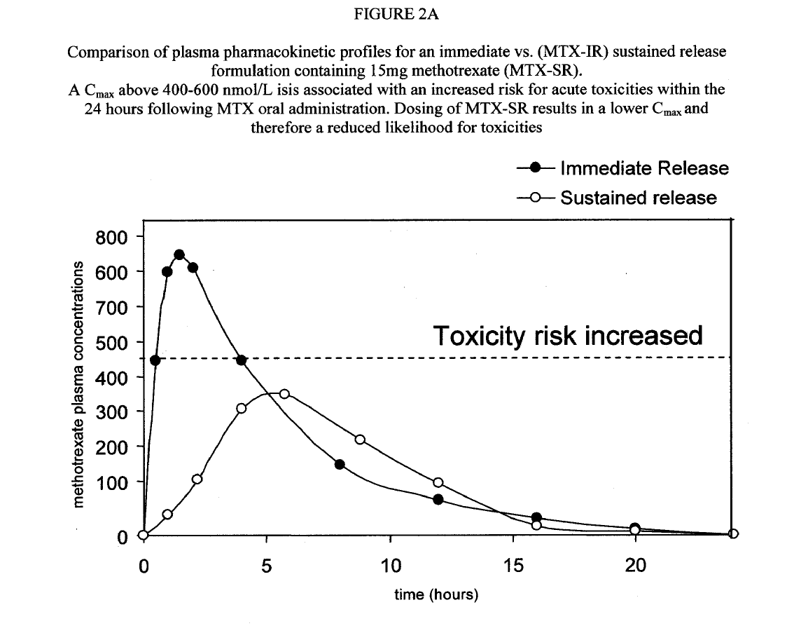

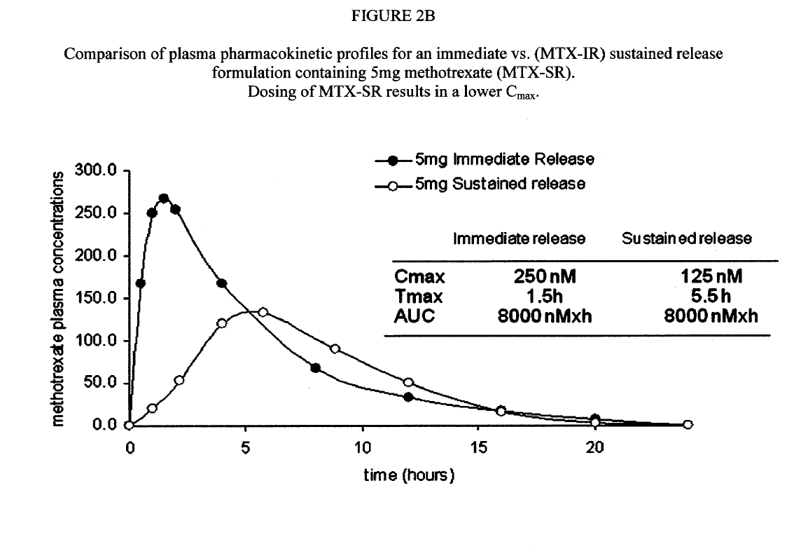

In some embodiments, the oral sustained released methotrexate composition will lower the maximal plasma concentration (Cₘₐₓ) of methotrexate, resulting in reduced toxicity. Exemplary pharmacokinetic profiles of such sustained release methotrexate formulations, compared to immediate release formulations, are presented in FIG. 2. These profiles illustrate 5 and 15 mg oral methotrexate administration to a patient with RA. As presented, the 15 mg sustained release formulation results in a two fold decrease in the Cₘₐₓ(from 800 nmol/L at 2 hours to 400 nmoVL at 6 hours) with no significant decrease in the area under the curve (AUC) (˜25,000 nmol.h/L).

Oral sustained release methotrexate compositions can be formulated by techniques described herein as well as by using techniques known in the art. The sustained release methotrexate technologies useful in various embodiments of the present invention include floating systems (also known as hydrodynamically balanced systems), swelling and expanding systems, bioadhesive systems, modified-shape systems, high-density systems and pharmacological delayed gastric emptying devices. Floating systems comprise buoyant preparations including hollow microspheres of gel forming or swellable cellulose type hydrocolloids, polysaccharides, and matrix forming polymers such as polycarbonate, polyacrylate, polymethacrylate and polystyrene. In one embodiment, methotrexate is combined with a gel-forming hydrocolloid that swells in contact with gastric fluid after oral administration and acts as a reservoir for sustained release of methotrexate. Other polymers can also be used that release methotrexate by osmotic pressure. For example, in bioadhesive systems, methotrexate is associated with polymeric nanoparticulate systems (or small particles in the micron size range). In one embodiment, the polymeric material is a non-swellable and hydrophobic polymer, such as poly(alkyl cyanoacrylate) or poly(lactic acid). In this miniature delivery system, methotrexate is released by osmotic pressure at an adjustable rate.

### Combinations of Methotrexate and Folic Acid

In some embodiments, the sustained-release methotrexate composition is administered with folic acid (and derivatives of folic acid). Folic acid (and derivatives of folic acid) can prevent some of the side effects associated with methotrexate. Although folic acid is considered a safe vitamin, the FDA recommends an upper tolerable dosage of 1000 micrograms per day. However, the amount of the folic acid derivative in the present invention can range from 1-10 mgs. In some embodiments, the folic acid is present in an amount of about 1 mg, or about 2.5 mgs, or about 5 mgs, or about 7 mgs. Folic acid derivatives useful in the present invention include folic acid, 5-methyltetrahydrofolic acid, and folinic acid.

In some embodiments, a Binary Release system may also be used to provide controlled release of methotrexate and a folate in a single formulation. One such system is a Quick Slow Release system that provides a quick burst of folate release followed by a constant rate of release of methotrexate over an extended period of time. Alternatively, in other embodiments, a Slow Quick release device may be used that provides an initial constant rate of release of methotrexate, followed by a quick release of folate. In another embodiment, a Positioned Release system is used which delivers methotrexate in the upper part of the intestine and folate in the colon. Lastly, a Delayed Release system may be used. This provides an immediate release of folate, followed by a release of methotrexate after a lag time of about 6 hours.

Tablets having distinct layers may be formulated using methods described herein as well as those know in the art. For example, bilayer tablets in which the folate is released prior to the methotrexate are provided in which the outer layer comprises the folate, and in which the inner layer comprises the methotrexate. These two layers may be separated by an inert or “spacer” later to further delay release of the methotrexate. In addition, the release of the folate and/or methotrexate may be delayed by the use of other coatings (e.g., enteric coatings). Polymers that may be used in table formulation to control the rate of release of methotrexate and/or folates include poly(urethanes), poly(siloxanes) poly(methyl methacrylate), poly(vinyl alcohol), poly(ethylene), poly(vinyl pyrrolidone), poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactides (PLA), polyglycolides (PGA,) poly(lactide-coglycolides) (PLGA), polyanhydrides, polyorthoesters, chitosan-alginate microcapsules with talc, microcrystalline cellulose, polymethacrylates, and pectin.

In one embodiment, the invention relates to compositions and pharmaceutical formulations for treatment of an autoimmune disorder, particularly rheumatoid arthritis, comprising a folate and sustained release methotrexate. The folate may be administered before or after the sustained release methotrexate. In one embodiment, the folate and sustained release methotrexate are provided as two separate entities, and administered separately. In another embodiment, the folate and sustained release methotrexate are present in the same formulation (e.g., in a multi-layer tablet or capsule). In another embodiment, the folate (e.g., 5-methyltetrahydrofolate) is administered first, followed by administration of methotrexate in either an immediate release or sustained release formulation. The folate and methotrexate may be administered separately, or may be present in the same formulation (e.g., tablet or capsule). Release of the sustained release methotrexate may begin, for example, 30 min, 1 h, 2 h, 3 h, 4 h, 5 h, 6 h, 7 h, 8 h, 9 h or 10 h before or after release of the folate, and may persist for hours or days. In addition, the release of either methotrexate, folate or both may be delayed, for example, by 30 min, 1 h, 2 h, 3 h, 4 h, 5 h, 6 h, 7 h, 8 h, 9 h or 10 h after administration.

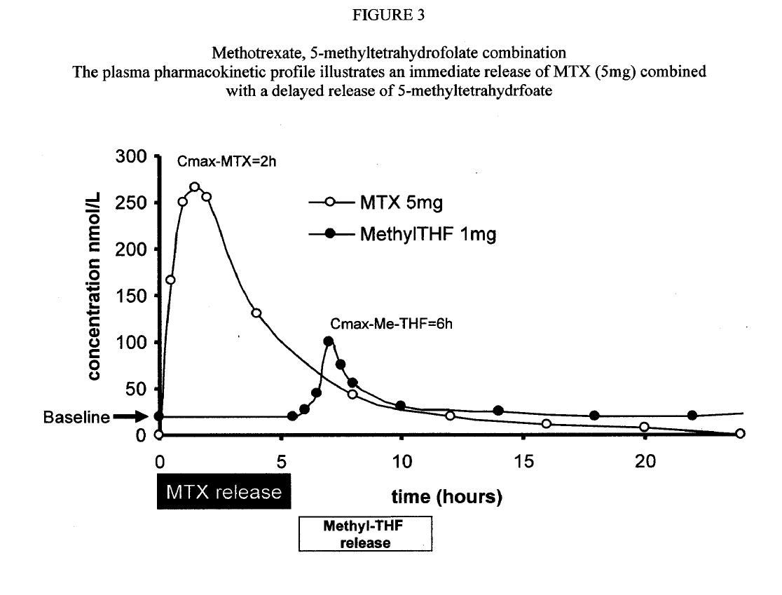

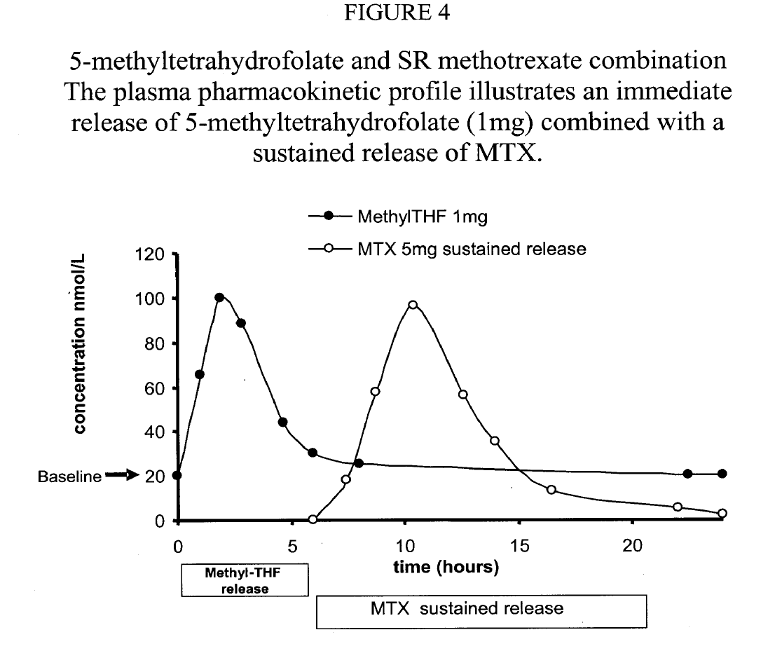

In another embodiment, methotrexate and 5-methyltetrahydrofolate are combined in a single oral formulation. As methotrexate and 5-methyltetrahydrofolate are preferentially absorbed in the upper part of the intestine by a pH dependant specialized carrier (Reduced Folate Carrier), a bilayer tablet that releases methotrexate, followed by 5-methyltetrahydrofolate, maximizes methotrexate absorption without significant interference from 5-methyltetrahydrofolate. This delivery method decreases methotrexate related acute toxicities because 5-methyltetrahydrofolate will provide the methyl moiety for the remethylation of homocysteine (increased following DHFR inhibition by methotrexate). A pharmacokinetic profile of a formulation containing 5 mg methotrexate with 1 mg 5-methyltetrahydrofolate formulation in described in FIG. 3. This release sequence may also be reversed by administration of 5-methyltetrahydrofolate first, followed by methotrexate, as the initial load of 5-methyltetrahydrofolate can counteract the subsequent increase in homocysteine following inhibition of DHFR by methotrexate. In addition, sustained release of methotrexate (as described above) in combination with 5-methyltetrahydrofolate will further reduce toxicity without affecting efficacy. A pharmacokinetic profile illustrating the combination of 5-methyltetrahydrofolate with a sustained release methotrexate is presented in FIG. 4.

When the drug is methotrexate, it can be present in an amount of between about 1 mg to about 50 mgs. For example, it can be present in an amount of about 1 mg, about 5 mgs, about 7.5 mgs, about 12.5 mgs, or about 15 mgs.

In some embodiments of the present invention, the methotrexate and folic acid derivative are together in a sustained-release dosage form.

In other embodiments of the present invention, the folic acid and methotrexate are in a bilayer tablet in which the folic acid layer is designed to be released immediately upon ingestion while the methotrexate layer is designed to be gastro-retentive and slowly release drug over time.

Gelling Agents

Matrix forming gelling agents include, but are not limited to Carbopol, Polycarbophil, hydroxypropyl methylcellulose (HPMC), methylcellulose, hydroxypropyl cellulose (HPC), carbomer, carboxy methylcellulose, polyethylene oxide (PolyOx®), gum tragacanth, gum acacia, guar gum, pectin, modified starch derivatives, xanthan gum, locust bean gum, Chitosan and its derivatives, sodium alginate, polyvinyl acetate (Kollidon-SR), polyethylene oxide and polyoxide. In some embodiments a combination of matrix forming gelling agents is used.

HPC has a viscosity in the range from 4,000 cps to about 100,000 cps. The concentration of the matrix forming gelling agent is from about 5-60 wt-% and more preferably in the range of 5-50 wt-%. In some embodiments, HPC is present in the composition in an amount of about 10 wt-%, about 20 wt-%, about 30 wt-%, about 40 wt-%, about 50 wt-%,

Agents that Float in Gastric Fluid

Various embodiments of the dosage forms described herein have bioadhesive characteristics so as they float in the upper region of the stomach, and they also attach to gastric mucosa resulting in gastric retention of at least 8 hours. As discussed infra, the floating ability can be enhanced by dispersing gas generating inorganic material (combination of one or more of bicarbonate and carbonate salt of Group I and Group II metal, including sodium, potassium, and calcium) throughout the dosage form. The concentration of a gas generating inorganic material is from about 1-30 wt-%, preferably in the range of about 2-25 wt-%, and more preferably in the range of about 3-20 wt-%.

Bioadhesive Agents

Bioadhesion is a surface phenomena in which a material may be of natural or synthetic origin, adheres or stick to biological surface, usually mucus membrane. The concept of bioadhesion is emerging as a potential application in drug delivery due to its applicability for prolonged GI residence time and better contact between drug and absorbing surface.

Many hydrophilic polymers adhere to mucosal surfaces as they attract water from the mucus gel layer adherent to the epithelial surface. This is the simplest mechanism of adhesion and has been defined as “adhesion by hydration”. Various kinds of adhesive forces, e.g. hydrogen bonding between the adherent polymer and the substrate, i.e. mucus, are involved in mucoadhesion at the molecular level. Carbopol polymers have been demonstrated to create a tenacious bond with the mucus membrane resulting in strong bioadhesion.

Polymers with bioadhesive characteristics include Carbopol (e.g., 71G, 937NF, 941NF from Noveon), Polycarbophil (Noveon), natural gums including guar gum and xanthan gum, chitosan and its derivatives (such as 5-methyl-pyrrolidone chitosan or MPC), hydroxypropyl cellulose (HPC, Klucel® by Aqualon), HPMC (Methocel® by Dow), polyethylene oxide (PolyOx® by Dow), sodium carboxymethyl cellulose, copolymers of acrylic acid or methacrylic acid, a poly(methyl vinyl ether/maleic anhydride) copolymer, pectin, alginic acid and its salt (e.g., sodium alginate), hyaluronic acid, gum tragacanth, and karaya gum. Many of these bioadhesive polymers also have floating and swelling characteristics.

Polymers known not to possess bioadhesive characteristics include lactose, microcrystalline cellulose, dicalcium phosphate, tricalcium phosphate and most hydrophobic polymers such as carnauba wax or other pharmaceutical grade lipophilic polymers.

The dosage forms described herein may comprise hydrophilic polymers such as cellulose derivatives, cross-linked polymers, or natural polymers such as gums and Chitosan.

In various embodiments, the polymers used to achieve the desired bioadhesive, floating and swelling properties are a combination of two or more polymers. In some embodiments, the combination of polymers is a combination of one or more cellulose polymers (including, but not limited to, HPMC, HPC, and CMC) and a cross-linked polymer such as polyacrylic acid polymer (including, but not limited to, Carbopol), polyvinyl acetate/polyvinyl pyrrolidone co-polymer (Kollidon-SR), or Eudragid RLPO. In some embodiments, the combination of polymers is a combination of HPC and PolyOx®. In some embodiments, the weight ratio of cellulose polymer to cross-linked polymer such as polyacrylic acid polymer is between about 1:1 to 1:5. In some embodiments, the weight ratio (cellulose derivative:polyacrylic acid polymer) is about 1:1.5, or about 1:2, or about 1:2.5, or about 1:3, or about 1:3.5, or about 1:4.

In alternate embodiments, a single polymer is used to achieve the desired bioadhesive, floating and swelling properties of the pharmaceutical composition. In some embodiments, this polymer is a cross-linked polyacrylic acid polymer such as Carbopol. In other embodiments, this polymer is a cellulose polymer such as HPMC, HPC, or CMC. In still other embodiments, the polymer is Kollidone-SR or Eudragit RS 30.

In some embodiments the weight % of the polymer(s) in the composition is between about 10-60 wt-%. In some embodiments the weight % is about 15 wt-%, or about 20 wt-%, or about 25 wt-%, or about 30 wt-%, or about 35 wt-%, or about 40 wt-%.

Other Components

The pharmaceutical compositions described herein may also comprise any other suitable ingredient well known to those skilled in the art of active substance formulation, such as adsorbents, fillers, antioxidants, buffering agents, colorants, flavorants, sweetening agents, antiadherents, lubricants, glidants, binders, diluents, disintegrants, tablet direct compression excipients, polishing agents.

Examples of such excipients include, but are not limited to magnesium stearate, calcium stearate, zinc stearate, powdered stearic acid, hydrogenated vegetable oils, talc, polyethylene glycol, mineral oil, an FD&C color, modified cellulose, lactose, gelatin, starch paste, acacia, tragacanth, povidone, colloidal silicon dioxide, talc, sodium lauryl sulfate, quaternary ammonium salts, mannitol, maltodextrin, dicalcium phosphate, tricalcium phosphate, sodium chloride, sodium sulfate, sodium phosphate, magnesium chloride, magnesium sulfate, magnesium phosphate, microcrystalline cellulose, sodium starch glycolate, lactose, microcrystalline cellulose, sucrose, glucose, calcium carbonate, colloidal anhydrous silica, waxes, hydrogenated castor oil, starch, polyvinyl pyrrolidone and combinations thereof.

Diluents, such as maltodextrin, lactose and derivatives thereof or dicalcium dihydrogen phosphate, microcrystalline cellulose and its compound products such as the combination of microcrystalline cellulose and colloidal silicon dioxide (Prosolv SMCC 90D from JRS), starches and derivatives thereof such as pregelatinised starches may be used in the dosage forms described herein.

A film coating may also be included on the outer surface of the dosage form for reasons other than a loading dose. The coating may thus serve an aesthetic function or a protective function, or it may make the dosage form easier to swallow or mask the taste of the drug.

For oral administration, dosage forms can be formulated readily by combining the active compounds with pharmaceutically acceptable carriers well known in the art. Such carriers enable the compounds of the invention to be formulated as tablets, pills, and the like, for oral ingestion by a patient to be treated. Pharmaceutical preparations for oral use can be obtained by mixing one or more solid excipient with pharmaceutical combination of the invention, optionally grinding the resulting mixture, and processing the mixture of granules, after adding suitable auxiliaries, if desired, to obtain tablets. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Tablet cores may be provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablet coatings for identification or to characterize different combinations of active compound doses.

Oral Dosing

Pharmaceutical carriers for hydrophobic compounds include a cosolvent system comprising benzyl alcohol, a nonpolar surfactant, a water-miscible organic polymer, and an aqueous phase. A common cosolvent system used is the VPD co-solvent system, which is a solution of 3% w/v benzyl alcohol, 8% w/v of the nonpolar surfactant POLYSORBATE 80™ and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of POLYSORBATE 80™; the fraction size of polyethylene glycol may be varied; other biocompatible polymers may replace polyethylene glycol, e.g., polyvinyl pyrrolidone; and other sugars or polysaccharides may substitute for dextrose.

Other delivery systems for hydrophobic pharmaceutical compounds include liposomes, emulsions and certain organic solvents such as dimethylsulfoxide, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Many of the active compounds used in the pharmaceutical compositions described herein may be provided as salts with pharmaceutically compatible counterions. Pharmaceutically compatible salts may be formed with many acids, including but not limited to hydrochloric, sulfuric, acetic, lactic, tartaric, malic, succinic, etc. or with bases, such as the hydroxide, carbonate or bicarbonate of a pharmaceutically acceptable metal cation (e.g. lithium, sodium, potassium, calcium, magnesium, and aluminum), with ammonia, or with a pharmaceutically acceptable organic primary, secondary or tertiary amine. Illustrative examples of some of the bases that can be used include sodium hydroxide, potassium hydroxide, choline hydroxide, sodium carbonate, N⁺(C₁₋₄ alkyl)₄, and the like. Salts tend to be more soluble in aqueous or other protonic solvents than are the corresponding free acid or base forms

Preparing the Formulation

The dosage forms as described herein can be prepared by common tableting methods that involve mixing, comminution, and fabrication steps commonly practiced by and well known to those skilled in the art of manufacturing drug formulations. Examples of such techniques are:
(1) Direct compression using appropriate punches and dies, typically fitted to a suitable rotary tablet press;
(2) Injection or compression molding;
(3) Granulation by fluid bed, by low or high shear granulation, or by roller compaction, followed by compression; and
(4) Extrusion of a paste into a mold or to an extrudate to be cut into lengths.

When direct compression is used, the addition of lubricants may be helpful to promote powder flow and to prevent breaking of the tablet (capping) when the pressure is relieved. Examples of typical lubricants are magnesium stearate, stearic acid and hydrogenated vegetable oils, such as, though not limited to hydrogenated and refined triglycerides of stearic and palmitic acids. Additional excipients may be added as granulating aids, binders, additives to enhance powder flowability, tablet hardness, and tablet friability and to reduce adherence to the die wall. Other fillers and binders include, but are not limited to, lactose (anhydrous or monohydrate), maltodextrins, sugars, starches, and other common pharmaceutical excipients. These additional excipients may constitute from 1% to 50% by weight, and in some cases more, of the tablet.

The pharmaceutical compositions described herein may be manufactured in a manner that is itself known, e.g., by means of conventional mixing, dissolving, granulating, dragee-making, levigating, encapsulating, entrapping or tabletting processes. In some embodiments, the pharmaceutical compositions comprise methotrexate and folate wherein the methotrexate and folate may be formulated together in the same composition or may be formulated separately. For example, the methotrexate may be provided in a first tablet, and the folate may be provided in a separate tablet. If more than one folate is included in the composition, they may also be formulated together or separately.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art; e.g., in Remington's Pharmaceutical Sciences.

Presentation of the Dosage Form

The pharmaceutical compositions described herein may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound of the invention formulated in a compatible pharmaceutical carrier may also be prepared, placed in an appropriate container, and labeled for treatment of an indicated condition.

The pharmaceutical compositions and methods described herein provide kits for the treatment of disorders, such as the ones described herein. These kits comprise a first and second agent or pharmaceutical compositions thereof in a container and, optionally, instructions teaching the use of the kit according to the various methods and approaches described herein. Such kits optionally include information, such as scientific literature references, package insert materials, clinical trial results, and/or summaries of these and the like, which indicate or establish the activities and/or advantages of the composition, and/or which describe dosing, administration, side effects, drug interactions, disease state for which the composition is to be administered, or other information useful to the health care provider. Such information may be based on the results of various studies, for example, studies using experimental animals involving in vivo models and studies based on human clinical trials. In various embodiments, the kits described herein can be provided, marketed and/or promoted to health providers, including physicians, nurses, pharmacists, formulary officials, and the like. Kits may, in some embodiments, be marketed directly to the consumer. In certain embodiments, the packaging material further comprises a container for housing the composition and optionally a label affixed to the container.

In one embodiment of the present invention, the kit described herein contains a therapeutically effect amount of first agent and a therapeutically effective amount of a second agent. In some embodiments, the second agent is a supplement. In certain embodiments, the present invention provides for a kit comprising a pharmaceutical composition, wherein the pharmaceutical composition contains either one of or both of the first and second agents.

In another embodiment, the kit comprises at least one first pharmaceutical composition and at least one second pharmaceutical composition. The first pharmaceutical composition contains a therapeutically effective amount of the first agent and the second pharmaceutical composition contains a therapeutically effective amount of the second agent. In some embodiments, the second agent is a supplement. In some embodiments, the first pharmaceutical composition is a sustained release formulation comprising methotrexate and the second pharmaceutical composition comprises a folate. In some embodiments, the amount of folate is such that the patient being treated with methotrexate is not being “over-rescued” with the folate supplement. In some embodiments, the first pharmaceutical composition does not contain a therapeutically effective amount of the second agent. In certain embodiments, the second pharmaceutical composition does not contain a therapeutically effective amount of the first agent. In other embodiments, the first pharmaceutical composition contains a therapeutically effective amount of the first agent and a therapeutically effective amount of the second agent. In still other embodiments, the second pharmaceutical composition contains a therapeutically effective amount of the second agent and a therapeutically effective amount of the first agent.

In a specific embodiment, the kit comprises (1) a first pharmaceutical composition that contains a therapeutically effective amount of a first agent and a therapeutically effective amount of the second agent, and (2) a second pharmaceutical composition that contains a therapeutically effective amount of the second agent and does not contain a therapeutically effective amount of the first agent. In another specific embodiment, the kit comprises (1) a first pharmaceutical composition that contains a therapeutically effective amount of a first agent and does not contain a therapeutically effective amount of the second agent, and (2) a second pharmaceutical composition that contains a therapeutically effective amount of the second agent and a therapeutically effective amount of the first agent. In some embodiments, the second agent is a supplement.

In some embodiments, the kit comprises a first pharmaceutical composition that is visibly different from a second pharmaceutical composition. The visible differences may be for example shape, size, color, state (e.g. liquid/solid), physical markings (e.g. letters, numbers) or a combination of these and the like. In certain embodiments, the kit comprises a first pharmaceutical composition that is a first color and a second pharmaceutical composition that is a second color. In embodiments wherein the first and second colors are different, the different colors of the first and second pharmaceutical compositions is used, e.g., to distinguish between the first and second pharmaceutical compositions. In further embodiments, the third pharmaceutical composition is a third color.

In some embodiments, wherein the packaging material further comprises a container for housing the pharmaceutical composition, the kit comprises a first pharmaceutical composition that is in a different physical location within the kit from a second pharmaceutical composition. In some embodiments, the different physical locations containing the first and second pharmaceutical compositions comprise separately sealed individual compartments. In certain embodiments, the kit comprises a first pharmaceutical composition that is in a first separately sealed individual compartment and a second pharmaceutical composition that is in a second separately sealed individual compartment. In embodiments wherein the first and second compartments are separate, the different locations of the first and second pharmaceutical compositions are used, e.g., to distinguish between the first and second pharmaceutical compositions. In some embodiments, where discrimination between the first and second pharmaceutical compositions is desirable, the kit comprises first and second pharmaceutical compositions both of different color and located in different physical locations within the kit.

In some embodiments of the present invention, the kit described herein contains a first, sustained release pharmaceutical formulation comprising methotrexate, and a second pharmaceutical formulation comprising a folate.

Intended Recipients of the Formulations

The compositions described herein may be used to treat a variety of vertebrates such as birds and mammals. Mammals suitable for treatment using the compositions and methods described herein include humans, primates, dogs, cats, rabbits, guinea pigs, horses, pigs, cows, and the like. A mammal having an autoimmune disease (e.g., RA) or cancer is identified, followed by administration of a pharmaceutical composition of the present invention.

It will be understood by those of skill in the art that numerous and various modifications can be made without departing from the spirit of the present invention. Therefore, it should be clearly understood that the forms of the present invention are illustrative only and are not intended to limit the scope of the present invention.

### EXAMPLES

Monolithic modified-release dosage forms (MMR) comprising at least one active substance with increased gastric residence time are described. The combination of size and shape along with bioadhesiveness and floatation are used to restrict the MMR to the stomach.

The following examples provide illustrative methods for testing the effectiveness and safety of the dosage forms. These examples are provided for illustrative purposes only and are not intended to limit the scope of the invention described or claims provided herein.

### Materials

The materials used to prepare the dosage forms described herein were obtained as follows. Methotrexate, USP was purchased from Fermion (Espoo, Finland); Carbopol 71-G was purchased from Noveon (Cleveland, Ohio, USA); HPMC, NF (Benecel® MP874, hydroxypropylmethylcellulose) and HPC, NF (Klucel®-HXF, hydroxypropyl cellulose) were purchased from Aqualon (Wilmington, Del., USA); Kollidon®-SR (PVA-PVP co-polymer, polyvinyl acetate and povidone based matrix retarding polymer) was purchased from BASF (Ludwigshafen am Rhein, Rhineland-Palatinate, Germany); Directly compressible Dicalcium Phosphate, NF (Emcompress®) was purchased from JRS Pharma (Patterson, N.Y., USA); Maltodextrin was purchased from Grains Processing Corp. (GPC, Muscatine, Iowa); Magnesium Stearate NF was purchased from Spectrum Chemicals (Gardena, Calif., USA) and directly compressible anhydrous Lactose (DCL-21) was purchased from DMV (Veghel, The Netherlands). Sodium bicarbonate (USP-2 grade) was purchased from Church and Dwight (Princeton, N.J.). Avicel® brand of microcrystalline cellulose-NF was purchased from FMC (Philadelphia, Pa.).

### Example 1

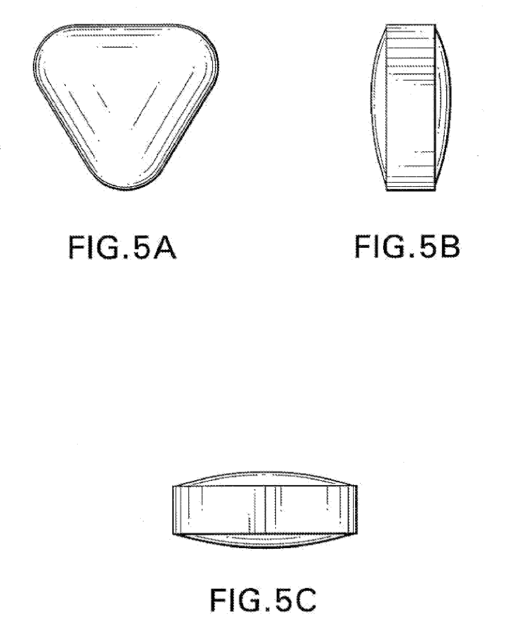

Dosage forms were prepared by common tableting methods, according to the following general procedure: Methotrexate and diluent (lactose, microcrystalline cellulose, maltodextrin or dicalcium phosphate) were manually mixed followed by mixing in poly bags. Bioadhesive polymers (Carbopol, HPC, HPMC, PolyOx®, etc.) were added and mixed well for at least 3 minutes. If used, sodium bicarbonate was added and mixed for at least 3 minutes. If used, magnesium stearate was added and mixed for 2 minutes. The resulting blends were compressed using a two station semi-automatic tablet press (RDB 410, Riddhi, Pharma, India) to form an equilateral triangular concave with rounded edge tooling shape, giving a slightly flattened tetrahedral shaped tablet, (see FIG. 5). Compression forces were recorded, if applicable. Approximately 20 tablets of each formulation were prepared. The resulting dosage form was assayed for bioadhesiveness, floating behavior, rate of medium uptake, and erosion/dissolution studies, as described below.

| Ingredient | mg/dose | % |
|---|---|---|
| Methotrexate | 5 | 1.2% |
| Carbopol (71G) | 100 | 24.3% |
| Lactose | 300 | 72.8% |
| Mg Stearate | 7 | 1.7% |
| Total | 412 | 100% |

### Example 2

The method of Example 1 was repeated using the following components:

| Ingredient | mg/dose | % |
|---|---|---|
| Methotrexate | 5 | 1.2% |
| Carbopol (71G) | 150 | 36.4% |
| Lactose | 250 | 60.7% |
| Mg Stearate | 7 | 1.7% |
| Total | 412 | 100% |

### Example 3

The method of Example 1 was repeated using the following components:

| Ingredient | mg/dose | % |
|---|---|---|
| Methotrexate | 5 | 1.2% |
| Carbopol (71G) | 150 | 36.4% |
| Sodium bicarbonate | 100 | 24.3% |
| Lactose | 150 | 36.4% |
| Mg Stearate | 7 | 1.7% |
| Total | 412 | 100% |

### Example 4

The method of Example 1 was repeated using the following components:

| Ingredient | mg/dose | % |
|---|---|---|
| Methotrexate | 5 | 1.2% |
| HPMC | 100 | 24.3% |
| Lactose | 300 | 72.8% |
| Mg Stearate | 7 | 1.7% |
| Total | 412 | 100% |

### Example 5

The method of Example 1 was repeated using the following components:

| Ingredient | mg/dose | % |
|---|---|---|
| Methotrexate | 5 | 1.2% |
| HPC (Klucel-H) | 100 | 24.3% |
| Lactose | 300 | 72.8% |
| Mg Stearate | 7 | 1.7% |
| Total | 412 | 100% |

### Example 6

The method of Example 1 was repeated using the following components:

| Ingredient | mg/dose | % |
|---|---|---|
| Methotrexate | 5 | 1.0% |
| Carbopol (71G) | 120 | 24.9% |
| HPC (Klucel-H) | 50 | 10.4% |
| Sodium bicarbonate | 100 | 20.7% |
| Dicalcium phosphate | 200 | 41.5% |
| Mg Stearate | 7 | 1.5% |
| Total | 482 | 100% |

### Example 7

The method of Example 1 was repeated using the following components:

| Ingredient | mg/dose | % |
|---|---|---|
| Methotrexate | 5 | 1.0% |
| Carbopol (71G) | 120 | 24.9% |
| HPC (Klucel-H) | 50 | 10.4% |
| Sodium bicarbonate | 100 | 20.7% |
| Lactose | 200 | 41.5% |
| Mg Stearate | 7 | 1.5% |
| Total | 482 | 100% |

### Example 8

The method of Example 1 was repeated using the following components:

| Ingredient | mg/dose | % |
|---|---|---|
| Methotrexate | 5 | 1.1% |
| Kollidon-SR | 150 | 32.5% |
| Lactose | 300 | 64.9% |
| Mg Stearate | 7 | 1.5% |
| Total | 462 | 100% |

### Example 9

The method of Example 1 was repeated using the following components:

| Ingredient | mg/dose | % |
|---|---|---|
| Methotrexate | 5 | 0.8% |
| Carbopol (71G) | 120 | 18.9% |
| Lactose | 250 | 39.4% |
| Dicalcium phosphate | 250 | 39.4% |
| Mg Stearate | 10 | 1.6% |
| Total | 635 | 100% |

### Example 10

The method of Example 1 was repeated using the following components:

| Ingredient | mg/dose | % |
|---|---|---|
| Methotrexate | 5 | 0.8% |
| Carbopol (71G) | 120 | 18.9% |
| HPC (Klucel-H) | 50 | 7.9% |
| Sodium bicarbonate | 50 | 7.9% |
| Lactose | 200 | 31.5% |
| Dicalcium phosphate | 200 | 31.5% |
| Mg Stearate | 10 | 1.6% |
| Total | 635 | 100% |

### Example 11

The method of Example 1 was repeated using the following components:

| Ingredient | mg/dose | % |
|---|---|---|
| Methotrexate | 5 | 0.8% |
| HPC (Klucel-H) | 120 | 18.9% |
| Lactose | 250 | 39.4% |
| Dicalcium phosphate | 250 | 39.4% |
| Mg Stearate | 10 | 1.6% |
| Total | 635 | 100% |

### Example 12

### PRO-515-CH

The method of Example 1 was repeated using the following components:

| Ingredient | mg/dose | % |
|---|---|---|
| Methotrexate | 5 | 0.8% |
| Carbopol (71G) | 120 | 18.9% |
| HPC (Klucel-H) | 50 | 7.9% |
| Sodium bicarbonate | 50 | 7.9% |
| Dicalcium phosphate | 200 | 31.5% |
| Maltodextrin | 200 | 31.5% |
| Mg Stearate | 10 | 1.6% |
| Total | 635 | 100% |

### Example 13

### PRO-515-CHSB

The method of Example 1 was repeated using the following components:

| Ingredient | mg/dose | % |
|---|---|---|
| Methotrexate | 5 | 0.8% |
| Carbopol (71G) | 160 | 25.2% |
| HPC (Klucel-H) | 50 | 7.9% |
| Sodium bicarbonate | 50 | 7.9% |
| Dicalcium phosphate | 200 | 31.5% |
| Maltodextrin | 160 | 25.2% |
| Mg Stearate | 10 | 1.6% |
| Total | 635 | 100% |

### Example 14

### PRO-515-H

The method of Example 1 was repeated using the following components:

| Ingredient | mg/dose | % |
|---|---|---|
| Methotrexate | 5 | 0.8% |
| HPC (Klucel-HXP) | 90 | 14.2% |
| Dicalcium phosphate | 180 | 28.3% |
| Maltodextrin | 350 | 55.1% |
| Mg Stearate | 10 | 1.6% |
| Total | 635 | 100% |

### Example 15

### PRO-515-HSB

The method of Example 1 is repeated using the following components:

| Ingredient | mg/dose | % |
|---|---|---|
| Methotrexate | 5 | 0.85% |
| PolyOx | 25 | 4.3% |
| HPC (Klucel-HXP) | 150 | 25.6% |
| Sodium Bicarbonate | 20 | 3.4% |
| MCC | 240 | 41.0% |
| Maltodextrin | 135 | 23.1% |
| Mg Stearate | 10 | 1.7% |
| Total | 585 | 100% |

### Example 16

### PRO-515-PHSB

The method of Example 1 is repeated using the following components:

| Ingredient | mg/dose | % |
|---|---|---|
| MTX-API | 5 | 0.8% |
| Polyox | 25 | 3.9% |
| HPC | 200 | 31.5% |
| Sodium bicarbonate | 20 | 3.1% |
| Maltodextrin | 135 | 21.3% |
| MCC | 240 | 37.8% |
| Mg Stearate | 10 | 1.6% |
| Total | 635 | 100% |

### Example 17

The following ingredients are blended together by dry mixing and made into dosage forms by direct compression at a fixed compression force. The dosage forms are diamond shaped with a triangular lateral length of 6-11 mm and with a vertical axis length of 2-7 mm, though other shapes may also be prepared. The resulting dosage from is assayed for bioadhesiveness, floating behavior, rate of medium uptake, erosion rate determination and dissolution studies, as described below.

| Ingredient | mg/dose | % |
|---|---|---|
| Methotrexate | 12.5 | 3.0% |
| Carbopol (71G) | 100 | 23.8% |
| Lactose | 300 | 71.5% |
| Mg Stearate | 7 | 1.7% |
| Total | 419.5 | 100.0% |

### Example 18

The method of Example 17 is repeated using the following components:

| Ingredient | mg/dose | % |
|---|---|---|
| Methotrexate | 12.5 | 3.0% |
| Carbopol (71G) | 150 | 35.8% |
| Lactose | 250 | 59.6% |
| Mg Stearate | 7 | 1.7% |
| Total | 419.5 | 100.0% |

### Example 19

The method of Example 17 is repeated using the following components:

| Ingredient | mg/dose | % |
|---|---|---|
| Methotrexate | 12.5 | 3.0% |
| Carbopol (71G) | 150 | 35.8% |
| Sodium bicarbonate (USP No. 1) | 100 | 23.8% |
| Lactose | 150 | 35.8% |
| Mg Stearate | 7 | 1.7% |
| Total | 419.5 | 100.0% |

### Example 20

The method of Example 17 is repeated using the following components:

| Ingredient | mg/dose | % |
|---|---|---|
| Methotrexate | 12.5 | 3.0% |
| HPMC | 100 | 23.8% |
| Lactose | 300 | 71.5% |
| Mg Stearate | 7 | 1.7% |
| Total | 419.5 | 100.0% |

### Example 21

The method of Example 17 is repeated using the following components:

| Ingredient | mg/dose | % |
|---|---|---|
| Methotrexate | 12.5 | 3.0% |
| HPC (Klucel-H) | 100 | 23.8% |
| Lactose | 300 | 71.5% |
| Mg Stearate | 7 | 1.7% |
| Total | 419.5 | 100.0% |

### Example 22

The method of Example 17 is repeated using the following components:

| Ingredient | mg/dose | % |
|---|---|---|
| Methotrexate | 12.5 | 3.0% |
| CMC | 150 | 35.8% |
| Lactose | 250 | 59.6% |
| Mg Stearate | 7 | 1.7% |
| Total | 419.5 | 100.0% |

### Example 23

The method of Example 17 is repeated using the following components:

| Ingredient | mg/dose | % |
|---|---|---|
| Methotrexate | 12.5 | 2.6% |
| Carbopol (71 G) | 120 | 24.5% |
| HPC (Klucel-H) | 50 | 10.2% |
| Sodium bicarbonate (USP No. 1) | 100 | 20.4% |
| Dicalcium phosphate | 200 | 40.9% |
| Mg Stearate | 7 | 1.4% |
| Total | 489.5 | 100.0% |

### Example 24

The method of Example 17 is repeated using the following components:

| Ingredient | mg/dose | % |
|---|---|---|
| Methotrexate | 12.5 | 2.6% |
| Carbopol (71G) | 120 | 24.5% |
| HPC (Klucel-H) | 50 | 10.2% |
| Sodium bicarbonate (USP No. 1) | 100 | 20.4% |
| Lactose | 200 | 40.9% |
| Mg Stearate | 7 | 1.4% |
| Total | 489.5 | 100.0% |

### Example 25

The method of Example 17 is repeated using the following components:

| Ingredient | mg/dose | % |
|---|---|---|
| Methotrexate | 12.5 | 2.6% |
| Kollidone-SR | 150 | 31.9% |
| Lactose | 300 | 63.9% |
| Mg Stearate | 7 | 1.4% |
| Total | 469.5 | 100.0% |

### Example 26

The method of Example 17 is repeated using the following components:

| Ingredient | mg/dose | % |
|---|---|---|
| Methotrexate | 12.5 | 2.7% |
| Kollidone-SR | 150 | 31.9% |
| Lactose | 300 | 63.9% |
| Mg Stearate | 7 | 1.5% |
| Total | 469.5 | 100.0% |

### Example 27

The method of Example 17 is repeated using the following components:

| Ingredient | mg/dose | % |
|---|---|---|
| Methotrexate | 12.5 | 2.7% |
| Eudragit RS 30 | 150 | 31.9% |
| Lactose | 300 | 63.9% |
| Mg Stearate | 7 | 1.5% |
| Total | 469.5 | 100.0% |

### Example 28

The method of Example 17 is repeated using the following components:

| Ingredient | mg/dose | % |
|---|---|---|
| Theophylline | 100 | 21.4% |
| Carbopol (71G) | 110 | 23.6% |
| Lactose | 250 | 53.5% |
| Mg Stearate | 7 | 1.5% |
| Total | 467 | 100.0% |

### Example 29

The method of Example 17 is repeated using the following components:

| Ingredient | mg/dose | % |
|---|---|---|
| Theophylline | 100 | 18.0% |
| Carbopol (71G) | 200 | 35.9% |
| Lactose | 250 | 44.9% |
| Mg Stearate | 7 | 1.3% |
| Total | 557 | 100.0% |

### Example 30

The method of Example 17 is repeated using the following components:

| Ingredient | mg/dose | % |
|---|---|---|
| Theophylline | 100 | 18.0% |
| Carbopol (71G) | 200 | 35.9% |
| Sodium bicarbonate (USP No. 1) | 100 | 18.0% |
| Lactose | 150 | 26.9% |
| Mg Stearate | 7 | 1.3% |
| Total | 557 | 100.0% |

### Example 31

The method of Example 17 is repeated using the following components:

| Ingredient | mg/dose | % |
|---|---|---|
| Theophylline | 100 | 18.3% |
| HPMC | 140 | 25.6% |
| Lactose | 300 | 54.8% |
| Mg Stearate | 7 | 1.3% |
| Total | 547 | 100.0% |

### Example 32

The method of Example 17 is repeated using the following components:

| Ingredient | mg/dose | % |
|---|---|---|
| Theophylline | 100 | 18.3% |
| HPC (Klucel-H) | 140 | 25.6% |
| Lactose | 300 | 54.8% |
| Mg Stearate | 7 | 1.3% |
| Total | 547 | 100.0% |

### Example 33

The method of Example 17 is repeated using the following components:

| Ingredient | mg/dose | % |
|---|---|---|
| Theophylline | 100 | 18.3% |
| CMC | 140 | 25.6% |
| Lactose | 300 | 54.8% |
| Mg Stearate | 7 | 1.3% |
| Total | 547 | 100.0% |

### Example 34

The method of Example 17 is repeated using the following components:

| Ingredient | mg/dose | % |
|---|---|---|
| Theophylline | 100 | 18.0% |
| Carbopol (71G) | 140 | 25.1% |
| HPC (Klucel-H) | 50 | 9.0% |
| Sodium bicarbonate (USP No. 1) | 110 | 19.7% |
| Dicalcium phosphate | 150 | 26.9% |
| Mg Stearate | 7 | 1.3% |
| Total | 557 | 100.0% |

### Example 35

The method of Example 17 is repeated using the following components:

| Ingredient | mg/dose | % |
|---|---|---|
| Theophylline | 100 | 18.0% |
| Carbopol (71G) | 140 | 25.1% |
| HPC (Klucel-H) | 50 | 9.0% |
| Sodium bicarbonate (USP No. 1) | 110 | 19.7% |
| Dicalcium phosphate | 150 | 26.9% |
| Mg Stearate | 7 | 1.3% |
| Total | 557 | 100.0% |

### Example 36

The method of Example 17 is repeated using the following components:

| Ingredient | mg/dose | % |
|---|---|---|
| Theophylline | 100 | 17.6% |
| Kollidone-SR | 180 | 31.7% |
| Lactose | 280 | 49.4% |
| Mg Stearate | 7 | 1.2% |
| Total | 567 | 100.0% |

### Example 37

The method of Example 17 is repeated using the following components:

| Ingredient | mg/dose | % |
|---|---|---|
| Theophylline | 100 | 17.6% |
| Eudragit RS 30 | 180 | 31.7% |
| Lactose | 280 | 49.4% |
| Mg Stearate | 7 | 1.2% |
| Total | 567 | 100.0% |

### Example 38

The method of Example 17 is repeated using the following components:

| Ingredient | mg/dose | % |
|---|---|---|
| Diclofenac-sodium | 100 | 21.4% |
| Carbopol (71G) | 110 | 23.6% |
| Lactose | 250 | 53.5% |
| Mg Stearate | 7 | 1.5% |
| Total | 467 | 100.0% |

### Example 39

The method of Example 17 is repeated using the following components:

| Ingredient | mg/dose | % |
|---|---|---|
| Diclofenac-sodium | 100 | 18.0% |
| Carbopol (71G) | 200 | 35.9% |
| Lactose | 250 | 44.9% |
| Mg Stearate | 7 | 1.3% |
| Total | 557 | 100.0% |

### Example 40

The method of Example 17 is repeated using the following components:

| Ingredient | mg/dose | % |
|---|---|---|
| Diclofenac-sodium | 100 | 18.0% |
| Carbopol (71G) | 200 | 35.9% |
| Sodium bicarbonate (USP No. 1) | 100 | 18.0% |
| Lactose | 150 | 26.9% |
| Mg Stearate | 7 | 1.3% |
| Total | 557 | 100.0% |

### Example 41

The method of Example 17 is repeated using the following components:

| Ingredient | mg/dose | % |
|---|---|---|
| Diclofenac-sodium | 100 | 18.3% |
| HPMC | 140 | 25.6% |
| Lactose | 300 | 54.8% |
| Mg Stearate | 7 | 1.3% |
| Total | 547 | 100.0% |

### Example 42

The method of Example 17 is repeated using the following components:

| Ingredient | mg/dose | % |
|---|---|---|
| Diclofenac-sodium | 100 | 18.3% |
| HPC (Klucel-H) | 140 | 25.6% |
| Lactose | 300 | 54.8% |
| Mg Stearate | 7 | 1.3% |
| Total | 547 | 100.0% |

### Example 43

The method of Example 17 is repeated using the following components:

| Ingredient | mg/dose | % |
|---|---|---|
| Diclofenac-sodium | 100 | 18.3% |
| CMC | 140 | 25.6% |
| Lactose | 300 | 54.8% |
| Mg Stearate | 7 | 1.3% |
| Total | 547 | 100.0% |

### Example 44

The method of Example 17 is repeated using the following components:

| Ingredient | mg/dose | % |
|---|---|---|
| Diclofenac-sodium | 100 | 18.0% |
| Carbopol (71G) | 140 | 25.1% |
| HPC (Klucel-H) | 50 | 9.0% |
| Sodium bicarbonate (USP No. 1) | 110 | 19.7% |
| Dicalcium phosphate | 150 | 26.9% |
| Mg Stearate | 7 | 1.3% |
| Total | 557 | 100.0% |

### Example 45

The method of Example 17 is repeated using the following components:

| Ingredient | mg/dose | % |
|---|---|---|
| Diclofenac-sodium | 100 | 18.0% |
| Carbopol (71G) | 140 | 25.1% |
| HPC (Klucel-H) | 50 | 9.0% |
| Sodium bicarbonate (USP No. 1) | 110 | 19.7% |
| Dicalcium phosphate | 150 | 26.9% |
| Mg Stearate | 7 | 1.3% |
| Total | 557 | 100.0% |

### Example 46

The method of Example 17 is repeated using the following components:

| Ingredient | mg/dose | % |
|---|---|---|
| Diclofenac-sodium | 100 | 17.6% |
| Kollidone-SR | 180 | 31.7% |
| Lactose | 280 | 49.4% |
| Mg Stearate | 7 | 1.2% |
| Total | 567 | 100.0% |

### Example 47

The method of Example 17 is repeated using the following components:

| Ingredient | mg/dose | % |
|---|---|---|
| Diclofenac-sodium | 100 | 17.6% |
| Eudragit RS 30 | 180 | 31.7% |
| Lactose | 280 | 49.4% |
| Mg Stearate | 7 | 1.2% |
| Total | 567 | 100.0% |

### Example 48

The method of Example 17 is repeated using the following components:

| | | | Amount |
|---|---|---|---|
| Ingredient | mg/dose | % | (g) |
| Methotrexate | 15 | 2.23% | 0.22 |
| Carbopol (71G) NF | 150 | 22.32% | 2.23 |
| DiCal Phosphate | 500 | 74.40% | 7.44 |
| Mg Stearate | 7 | 1.04% | 0.10 |
| Total | 672 | 100.0% | 10.0 |

### Example 49

The method of Example 17 is repeated using the following components:

| | | | Amount |
|---|---|---|---|
| Ingredient | mg/dose | % | (g) |
| Methotrexate | 15 | 1.99% | 0.20 |
| HPMC | 230 | 30.54% | 3.05 |
| DiCal Phosphate | 500 | 66.40% | 6.64 |
| Mg Stearate | 8 | 1.06% | 0.11 |
| Total | 753 | 100.0% | 10.0 |

### Example 50

The method of Example 17 is repeated using the following components:

| | | | Amount |
|---|---|---|---|
| Ingredient | mg/dose | % | (g) |
| Methotrexate | 15 | 1.99% | .20 |
| HPC (Klucel EXP) | 230 | 30.54% | 3.05 |
| DiCal Phosphate | 500 | 66.40% | 6.64 |
| Mg Stearate | 8 | 1.06% | 0.11 |
| Total | 753 | 100.0% | 10.0 |

### Example 51

The method of Example 17 is repeated using the following components:

| | | | Amount |
|---|---|---|---|
| Ingredient | mg/dose | % | (g) |
| Methotrexate | 15 | 2.07% | 0.21 |
| Xanthan Gum | 100 | 13.83% | 1.38 |
| HPC (Klucel EXP) | 100 | 13.83% | 1.38 |
| DiCal Phosphate | 500 | 69.16% | 6.92 |
| Mg Stearate | 8 | 1.11% | 0.11 |
| Total | 723 | 100.0% | 10.0 |

### Example 52

The method of Example 17 is repeated using the following components:

| | | | Amount |
|---|---|---|---|
| Ingredient | mg/dose | % | (g) |
| Methotrexate | 15 | 2.07% | 0.21 |
| Kollidon SR | 200 | 27.66% | 2.77 |
| DiCal Phosphate | 500 | 69.16% | 6.92 |
| Mg Stearate | 8 | 1.11% | 0.11 |
| Total | 723 | 100.0% | 10.0 |

### Example 53

### Bilayer Tablet of Methotrexate and Folic Acid

A bilayer tablet in which the folic acid layer is designed to be released immediately upon ingestion while the methotrexate layer is designed to be gastro-retentive and slowly release drug over time. While not limiting in nature, provided below are two examples of bilayer tablets useful in the present invention. Exemplary components for the two layers of the tablets are shown in the tables below. Each immediate release layer and sustained release layer blend are separately mixed. Methotrexate/Folic acid tablets are compressed using a tablet press with two powder feed systems that is capable of processing bilayer tablets, such as a Manesty bilayer tablet press.

### Example 53A

| | mg/dose | % Level |
|---|---|---|
| Sustained Release Layer | | |
| MTX | 5 | 1.08% |
| Carbopol (71G) | 150 | 32.47% |
| Sodium bicarbonate (USP | 100 | 21.65% |
| No. 1) | | |
| Lactose Microcrystalline | 200 | 43.29% |
| Cellulose | | |
| Mg Stearate | 7 | 1.52% |
| Total | 462 | 100.00% |
| Immediate Release Layer | | |
| Folic Acid | 2.5 | 1.22% |
| Lactose | 200 | 97.32% |
| Mg Stearate | 3 | 1.46% |
| Total | 205.5 | 100.00% |

### Example 53B

| | mg/dose | % Level |
|---|---|---|
| Sustained Release Layer | | |
| MTX | 10 | 3.17% |
| HPC-HXP | 100 | 31.75% |
| Dical Phosphate | 200 | 63.49% |
| Mg Stearate | 5 | 1.59% |
| Total | 315 | 100.00% |
| Immediate Release Layer | | |
| Folic Acid | 5 | 2.40% |
| Dical Phosphate | 200 | 96.15% |
| Mg Stearate | 3 | 1.44% |
| Total | 208 | 100.00% |

### Example 54

### Sustained-Release Tablet of Both Methotrexate and Folic Acid

In a manner analogous to that described in Example 17, methotrexate and folic acid are mixed homogeneously in the matrix tablet for sustained release of both MTX and folic acid over time. All ingredients are homogeneously mixed and compressed into a tablet.

### Example 54A

| Ingredient | mg/dose | % |
|---|---|---|
| Methotrexate | 10 | 2.69% |
| Folic acid | 5 | 1.34% |
| Carbopol (71G) | 150 | 40.32% |
| Sodium bicarbonate (USP No. 1) | 50 | 13.44% |
| Lactose | 150 | 40.32% |
| Mg Stearate | 7 | 1.88% |
| Total | 372 mg | 100.0% |

### Example 54B

| Ingredient | mg/dose | % |
|---|---|---|
| Methotrexate | 10 | 1.38% |
| Folic acid | 5 | 0.69% |
| Xanthan Gum | 100 | 13.83% |
| HPC (Klucel EXP) | 100 | 13.83% |
| DiCal Phosphate | 500 | 69.16% |
| Mg Stearate | 8 | 1.11% |
| Total | 723 | 100.0% |

### Example 55

### Thickness of Dosage Forms

The thickness of the tablets described in examples 1-14 was measured with a digital read-out Caliper (n=5; average values are shown).

| Example # | Thickness |
|---|---|
| 1 | 4.3 mm |
| 2 | 4.3 mm |
| 3 | 4.0 mm |
| 4 | 4.1 mm |
| 5 | 4.1 mm |
| 6 | 4.5 mm |
| 7 | 4.6 mm |
| 8 | 4.6 mm |
| 9 | 5.7 mm |
| 10 | 5.6 mm |
| 11 | 5.5 mm |
| 12 | 5.6 mm |
| 13 | 5.8 mm |
| 14 | 5.7 mm |

### Example 56

### Hardness of Dosage Forms

The hardness of the tablets described in examples 1-14 was measured with a Varian 200 tablet hardness tester (n=5; average values are shown).

| | Hardness |
|---|---|
| Example # | (mean, Kp) |
| 1 | 6.4 |
| 2 | 10.8 |
| 3 | 9.0 |
| 4 | 5.2 |
| 5 | 6.9 |
| 6 | 6.7 |
| 7 | 7.2 |
| 8 | 9.5 |
| 9 | 8.3 |
| 10 | 8.1 |
| 11 | 8.2 |
| 12 | 8.3 |
| 13 | 10.8 |
| 14 | 9.0 |

Hardness can be determined for other compositions described and claimed herein using the method described herein or similar methods known to those of skill in the art.

### Example 57

### Friability of Dosage Forms

The friability of the tablets described in examples 1-8 was not directly measurable due to small scale compression. The Friability of the tablets described in examples 9-14 were determined as follows. The weight of a minimum of 5 tablets was accurately measured. The tablets were placed in a Vankel friability tester for 100 revolutions at 25 rpm. The tablets were removed and reweighed. The friability (%) is calculated as follows:

$$\% friability = 100 \frac{( {w}_{i} - {w}_{f} )}{{w}_{i}}$$

where
wᵢ=initial weight of tablets (min n=5)
w_{f}=final weight of tablets (min n=5)

| Example # | Friability |
|---|---|
| 9 | 0.50% |
| 10 | 0.46% |
| 11 | 0.39% |
| 12 | 0.46% |
| 13 | 0.33% |
| 14 | 0.38% |

The tablets of examples 9-14 demonstrated no picking or lamination.

Friability can be determined for other compositions described and claimed herein using the method described herein or similar methods known to those of skill in the art.

### Example 58

### In Vitro Drug Release

In vitro drug release profiles of the matrix tablets prepared in examples 1-14 were determined by an in-vitro dissolution method, according to the Methotrexate-USP monograph method using a USP apparatus II (Paddle). The dissolution medium was 900 mL 0.1N hydrochloric acid at 37° C.; paddle speed 50 rpm, using 1 L capacity vessels. All experiments were performed in triplicate and average values were taken. Sample (10 mL) was withdrawn at predetermined time intervals (1 h, 2 h, 4 h, 6 h, 8 h, 10 h, 12 h, 18 h, 24 h, and infinity; after additional 1 h with increased paddle speed of 200 rpm), filtered and replaced by an equal volume of dissolution medium. Dissolution data were corrected for the dilution effect (see Hayton et al, Correction of perfusate for sample removal, J. Pharm. Sci. 1982, 71, 820-821). Samples were analyzed using a VK 8000 auto sampler, using 0.6 mL sampler cleaning volume and zero volume replacement. Samples were filtered online, using full Flow Filters™ (Poly ethylene Filter, 35 μm), and analyzed with a UV-Vis spectrophotometer at 306 nm. Drug dissolved at specified time periods is plotted as percent release versus time (hours).

In vitro drug release can be determined for other compositions described and claimed herein using the method described herein or similar methods known to those of skill in the art.

It was observed that the matrix tablets prepared in examples 3 and 7 floated to the surface of the dissolution vessel almost immediately, and slowly started to disintegrate. The other formulations slowly floated to the upper middle portion of the dissolution vessels and remained afloat longer without disintegration.

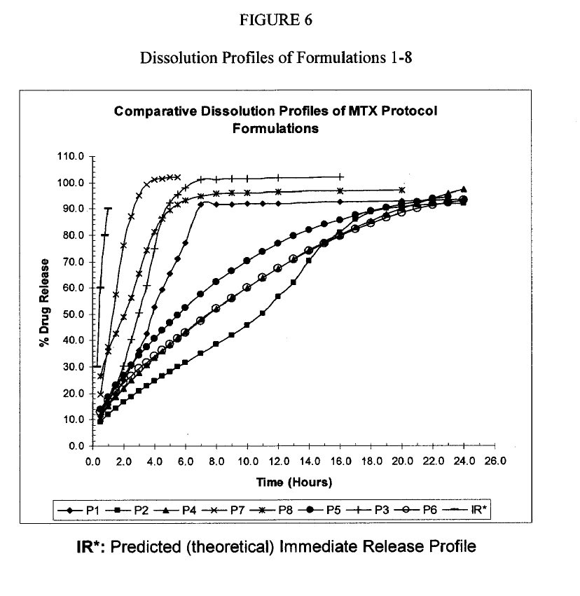

The dissolution profiles of formulations of examples 1-8 are shown in FIG. 6.

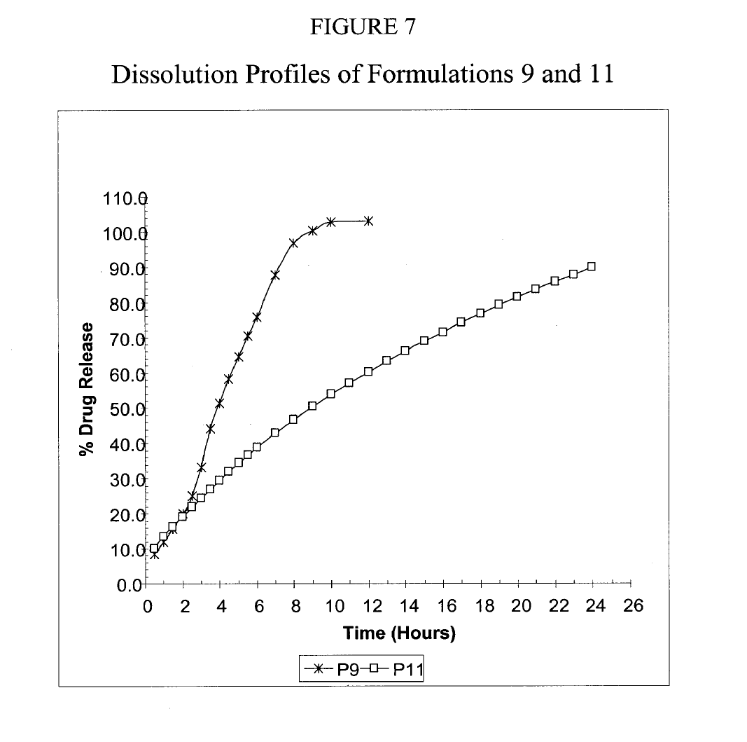

The dissolution profiles of formulations of examples 9 and 11 are shown in FIG. 7.

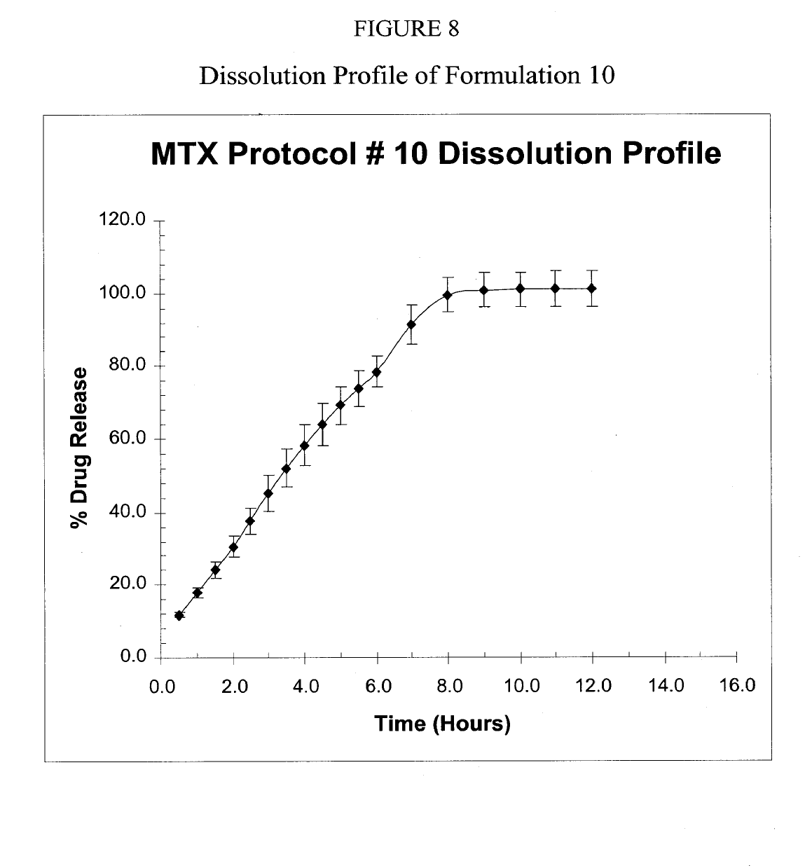

The dissolution profile of formulation of example 10 is shown in FIG. 8.

Drug release times (>80%) methotrexate) were calculated and are given in the table below:

| | Drug release | |
|---|---|---|
| Example # | time | |
| 1 | 7 | h |
| 2 | >26 | h |
| 3 | 3 | h |
| 4 | 18-20 | h |
| 5 | 18-20 | h |
| 6 | 24 | h |
| 7 | 3 | h |
| 8 | 6 | h |
| 9 | 8 | h |
| 10 | 8 | h |
| 11 | 24 | h |
| 12 | 6 | h |
| 13 | 12 | h |
| 14 | 16 | h |
| 15 | 18 | h |
| 16 | 12 | h |

Several factors affect the release profile of the formulations 1-16.

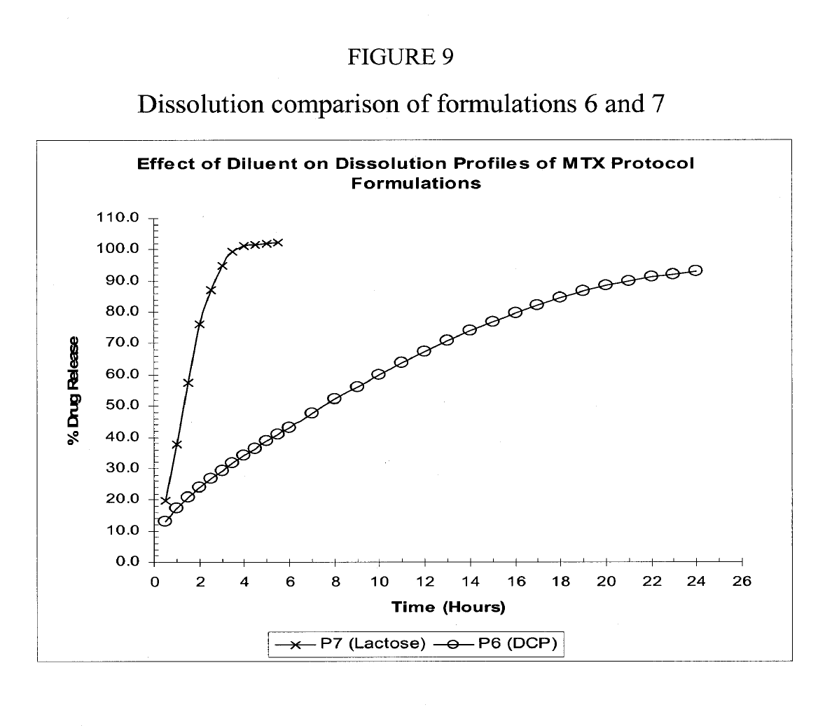

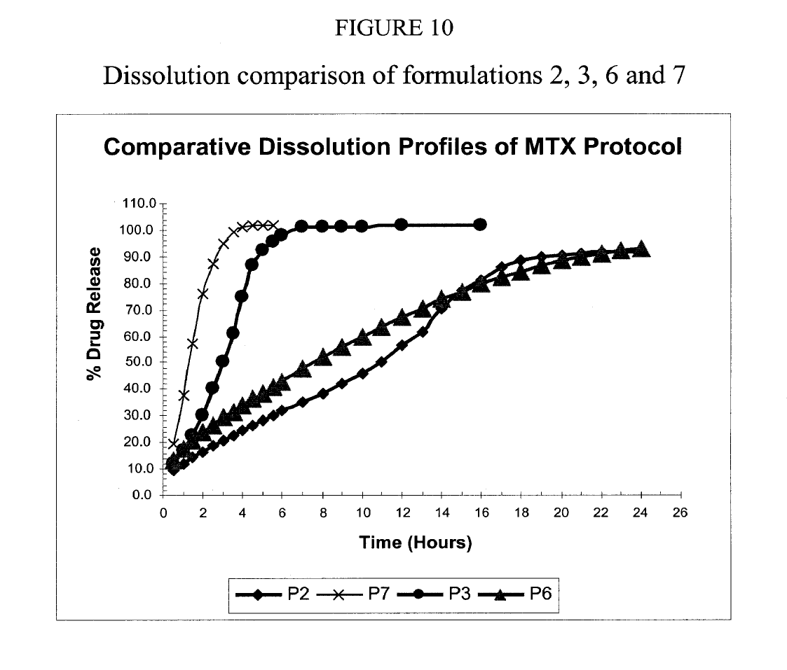

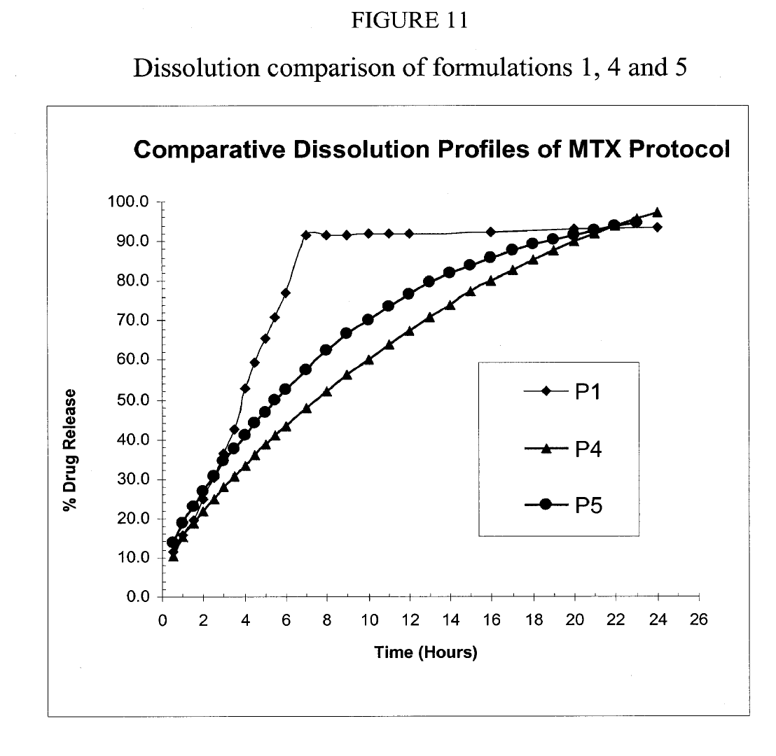

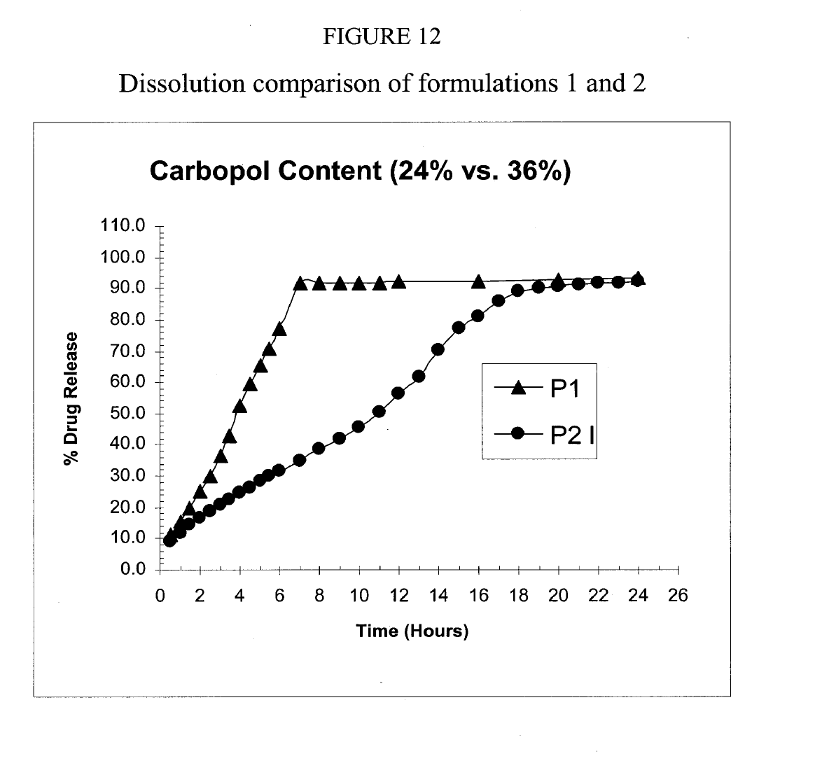

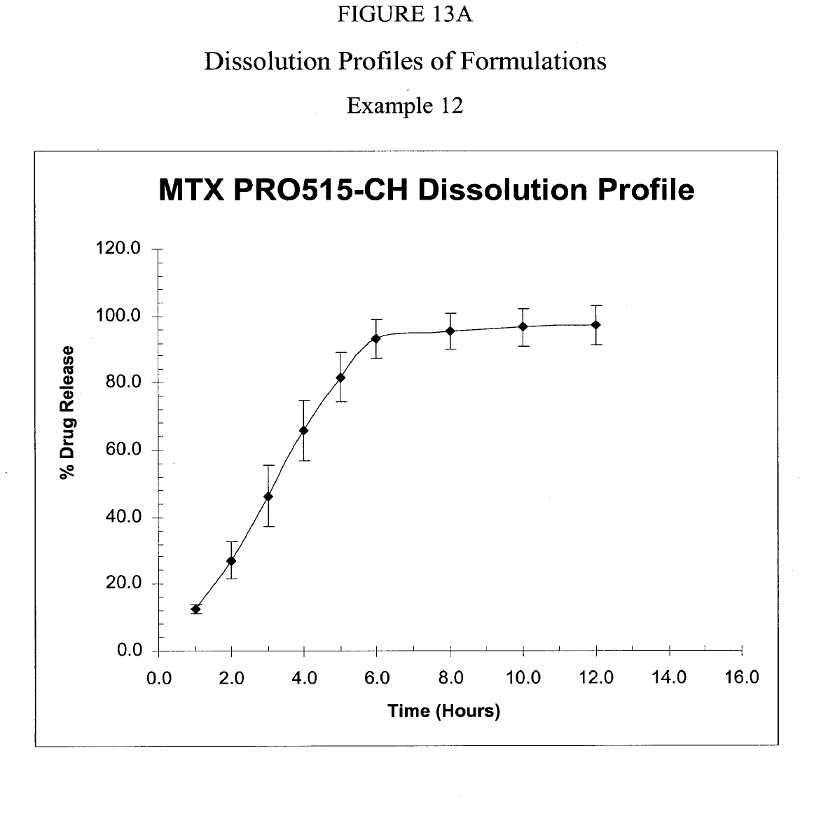

i) Effect of diluent: lactose vs. dicalcium phosphate. The comparison of dissolution profiles of formulations 6 and 7 (dicalcium phosphate and lactose diluents, respectively) are shown in FIG. 9. Further, replacing lactose with maltodextrin, results in slightly faster drug release, possibly due to higher water solubility of maltodextrin compared to lactose (>3 times higher).
ii) Effect of Sodium Bicarbonate. The comparison of dissolution profiles of formulations 2, 3, 6 and 7 are shown in FIG. 10.
iii) Effect of Hydrophilic Polymers. The comparison of dissolution profiles of formulations 1, 4 and 5 (containing different hydrophilic polymers in the same amounts) are shown in FIG. 11. Higher hydrophilic polymer (Carbopol/HPC) content results in faster drug release. Note, HPC and HPMC are both hydrophilic substituted cellulose and they have similar drug release mechanisms via swelling, diffusion and eroding, (see Levina, M. Influence of Fillers, Compression Force, Film Coatings and Storage Conditions on Performance of Hypromellose Matrices, Drug Delivery Technology).
iv) Effect of Carbopol quantity. The comparison of dissolution profiles of formulations 1 and 2 (containing different amounts of carbopol) are shown in FIG. 12.

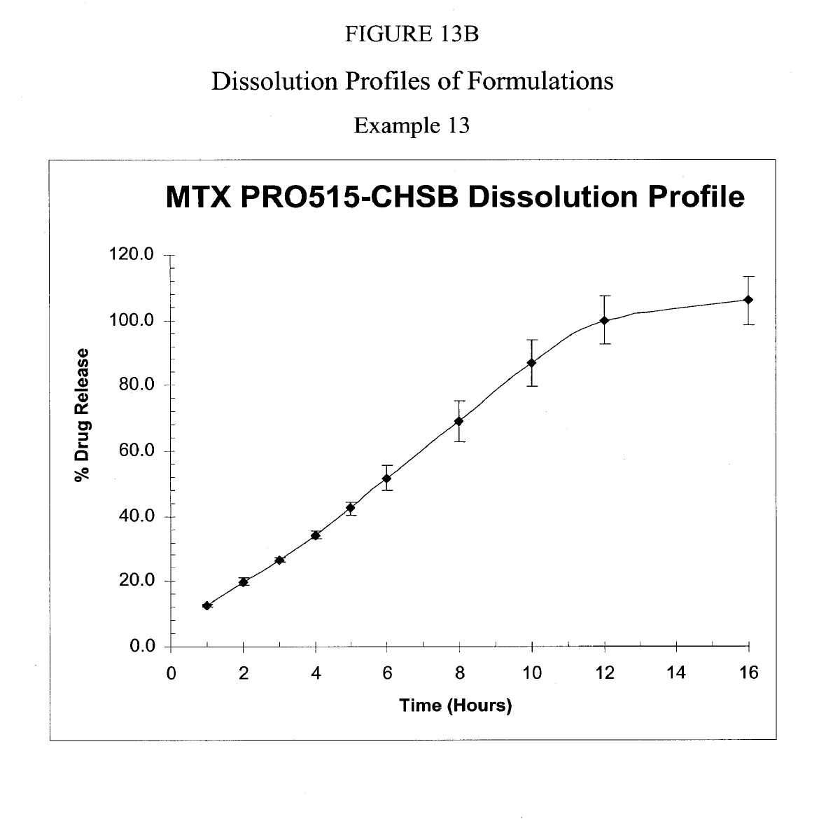

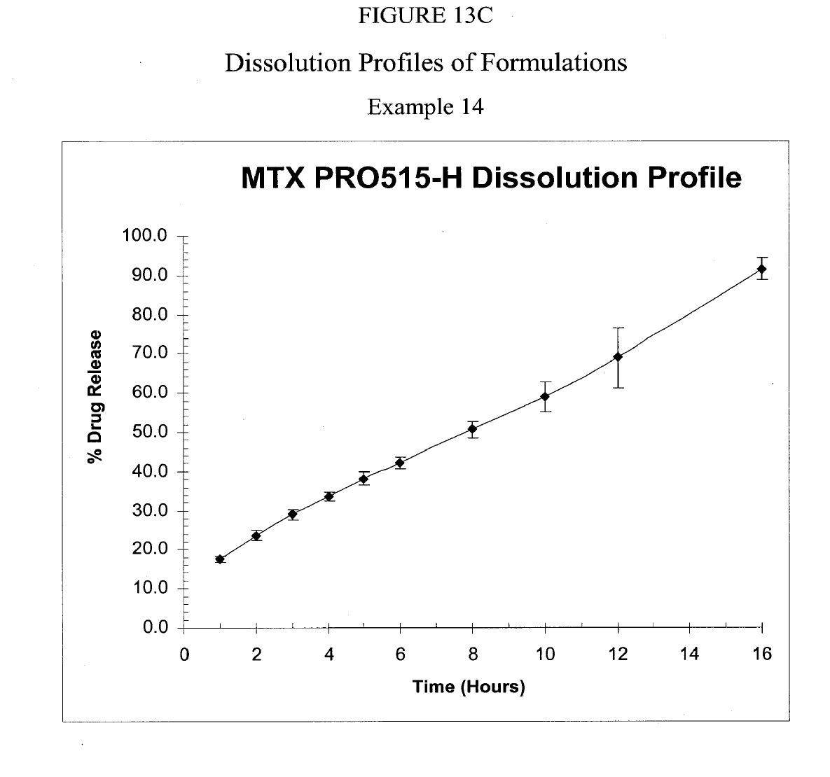

The dissolution profiles of formulations 12, 13 and 14 are shown in FIGS. 13A, 13B and 13C, respectively.

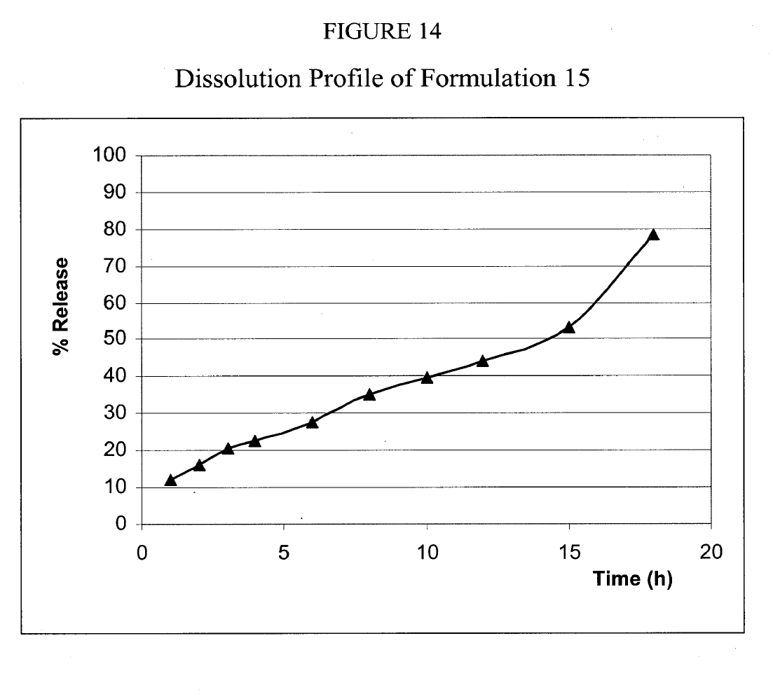

The dissolution profiles of formulation 15 is shown in FIG. 14.

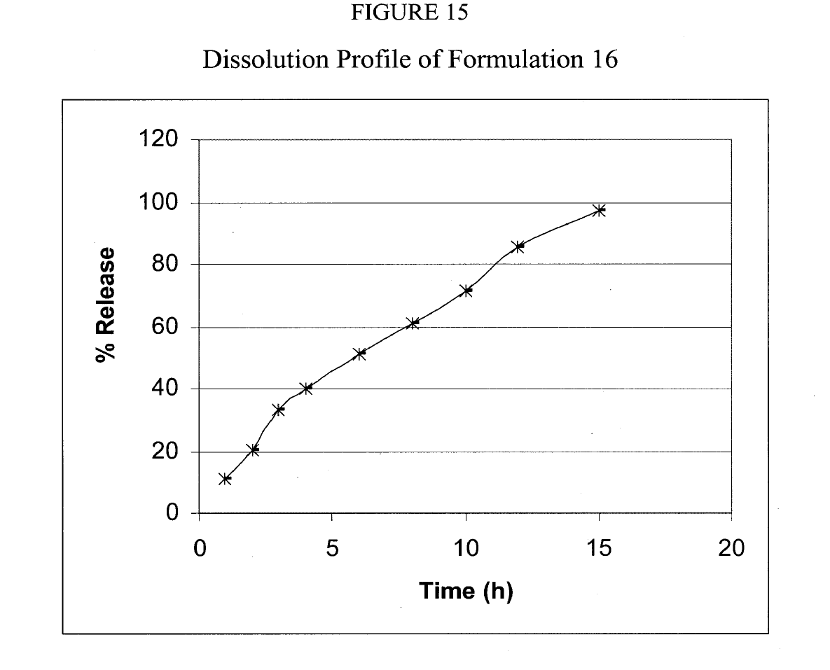

The dissolution profiles of formulation 16 is shown in FIG. 15.

### Example 59

### Rate of Medium Uptake and Erosion Rate Determination

The rate of medium uptake and erosion rates were determined for the tablets described in examples 12, 13 and 14 after immersion in the medium, according to the general procedure described below (see Gerogiannis, Drug De. Ind. Pharm. 1993, 19 (9), 1061-1081). All experiments were performed in triplicate; three different samples were measured for each time point, and fresh samples were used for each individual time point. Samples were weighed and placed in dissolution vessels (USP type I or II dissolution apparatus) containing simulated gastric fluid (SGF) at pH 1.0-1.2 at 37±0.2° C. After the allotted time interval (1 h, 2 h, 3 h, 5 h, 8 h, 12 h, and 24 h) each dissolution basket was withdrawn, blotted to remove excess water from the tablets, and the dosage form dimensions (length and width) measured in triplicate. The tablets were then weighed (within 10 minutes of the withdrawal from the medium to prevent drying out) on an analytical balance. The wet samples were then dried in an oven at >100° C. for at least 24 hours, allowed to cool in a desiccator, and weighed to constant weight (final dry weight). The increase in weight of the wet mass represents the medium uptake. The increase in weight due to absorbed liquid (Q) was estimated according to equation 1.

$$Q = 100 \frac{( {W}_{w} - {W}_{f} )}{{W}_{f}} \qquad ( equation 1 )$$

where
Q is the increase in weight due to absorbed liquid
W_{w} is the mass of the hydrated sample (before drying)
W_{f}) is the final mass of the same dried, partially eroded sample
The percentage erosion (E) was estimated according to equation 2.

$$E = 100 \frac{( {W}_{i} - {W}_{f} )}{{W}_{i}} \qquad ( Equation 2 )$$

where
E is the percentage erosion
Wᵢ is the initial starting dry weight
W_{f} is the final mass of the same dried, partially eroded sample.

Swelling and erosion studies can be performed on other compositions described and claimed herein using the method described herein or similar methods known to those of skill in the art.

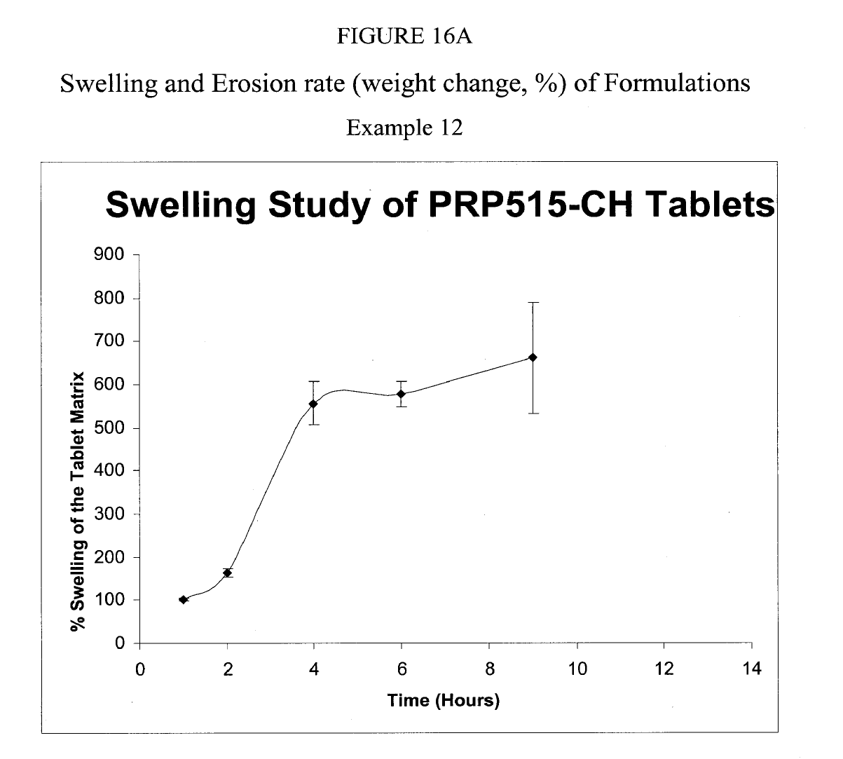

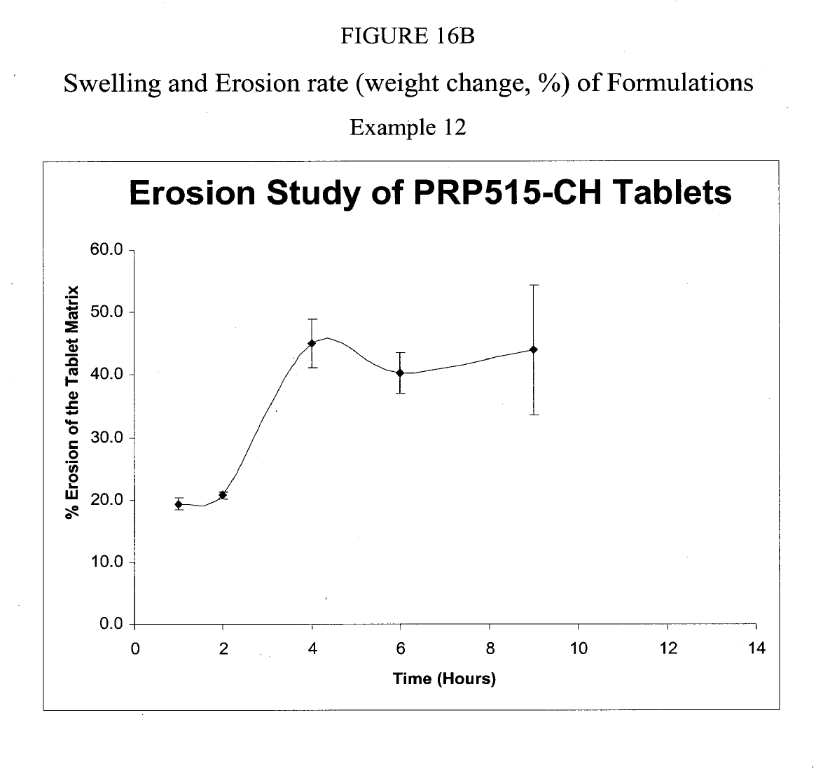

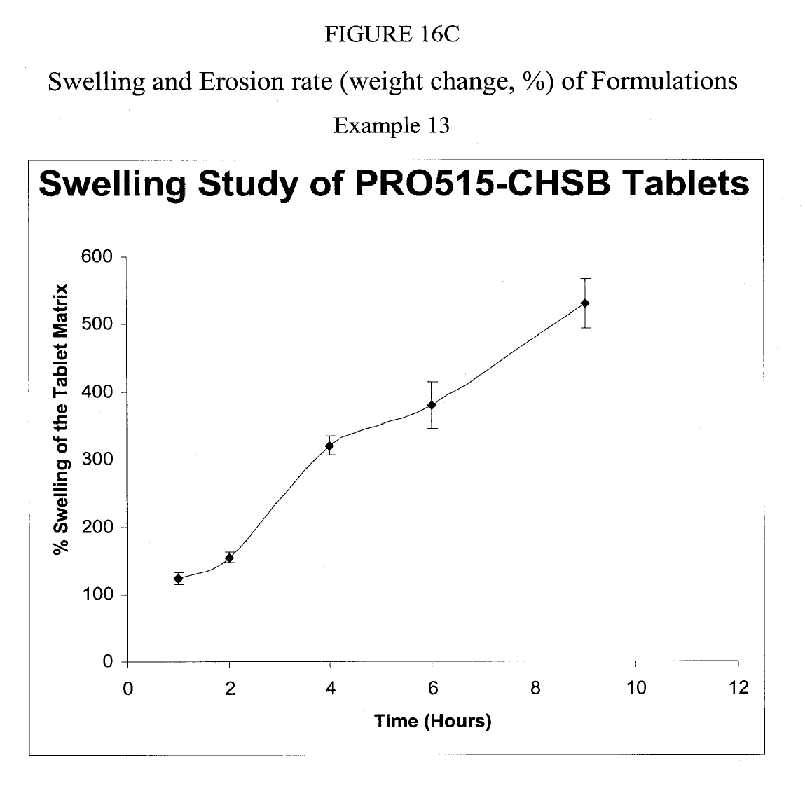

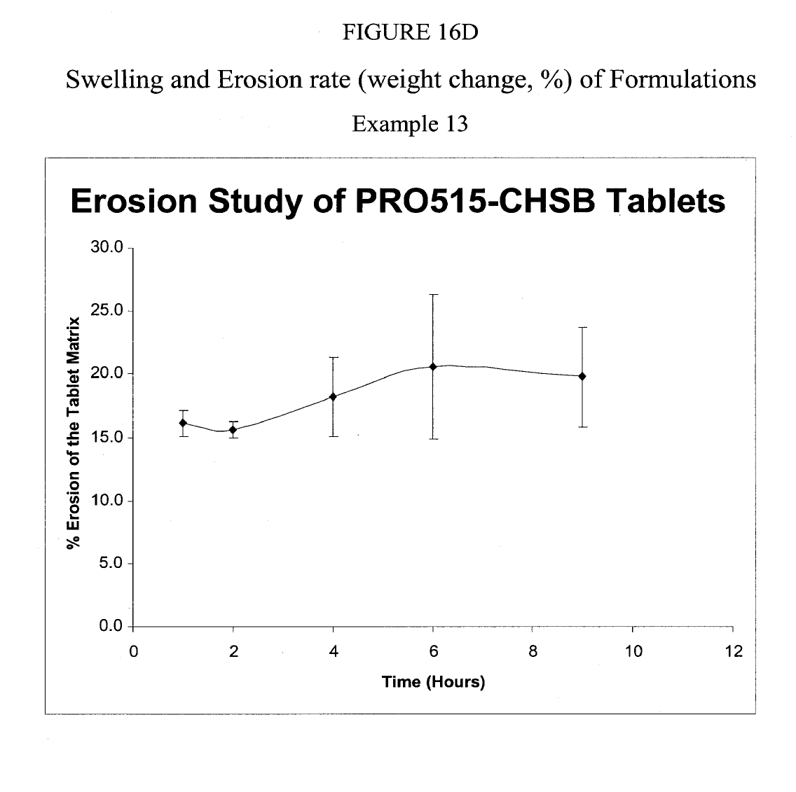

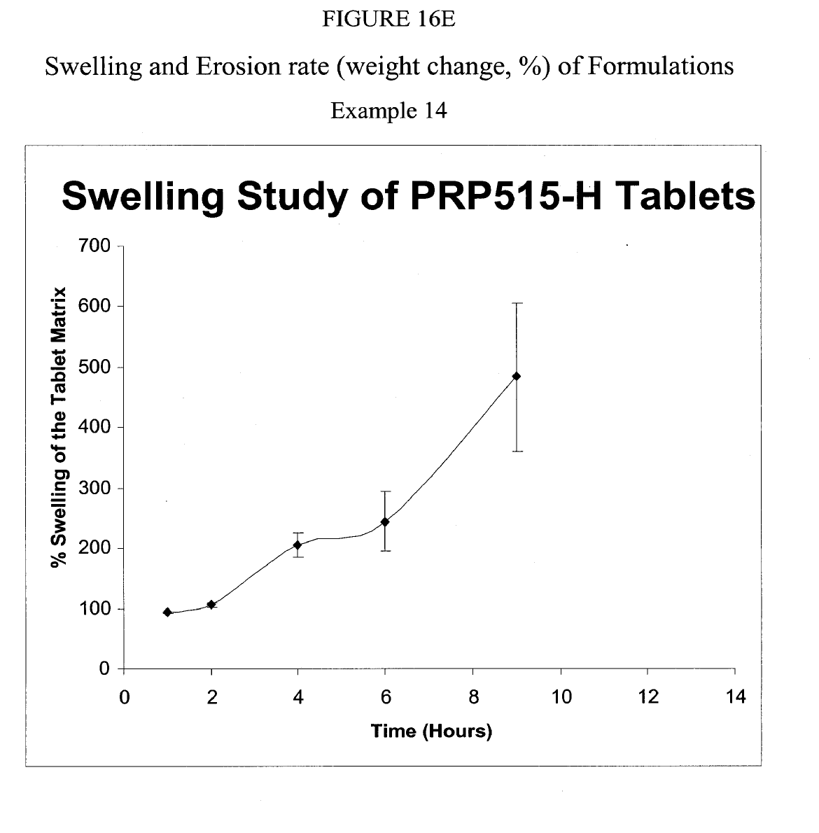

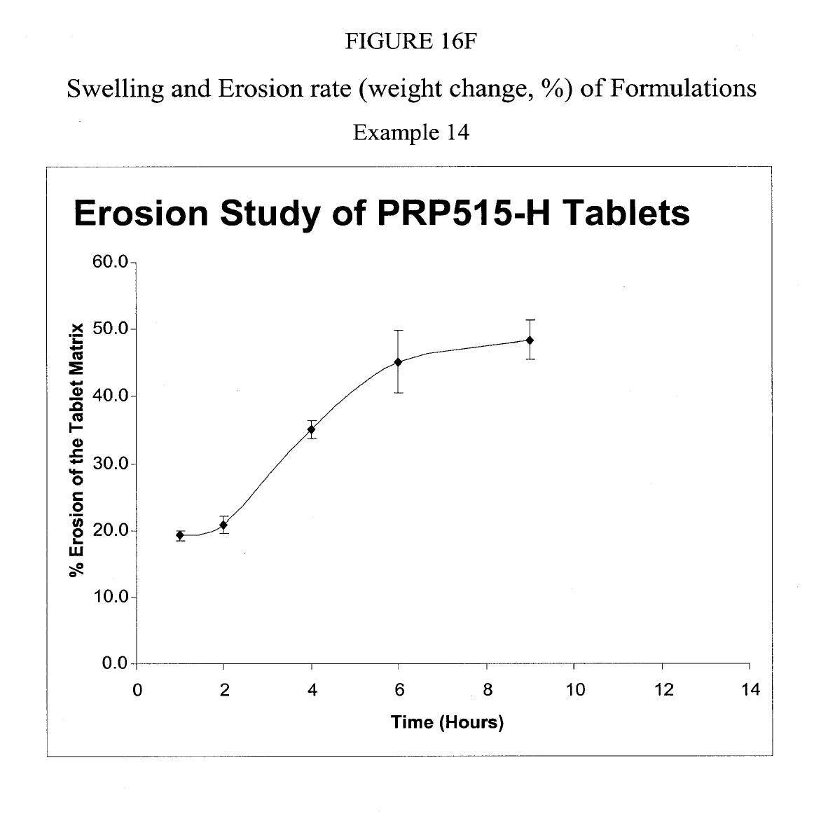

The results of swelling and erosion (weight change, %) studies on the tablets of examples 12, 13 and 14 are shown below and in FIGS. 16A-16F.

### Tablet of Example 12

Tablet size Change

| | Initial Tablet Dimensions | | | | Final Tablet Dimensions | | | |
|---|---|---|---|---|---|---|---|---|
| | (mm) n = 3 | | | | (mm) n = 3 | | | |
| Time | Mean | SD | Mean | SD | Mean | SD | Mean | SD |
| (Hrs) | Length | (%) | Width | (%) | Length | (%) | Width | (%) |
| 1 | 11.12 | 0.07 | 11.58 | 0.12 | 13.82 | 0.33 | 14.51 | 0.29 |
| 2 | 11.09 | 0.15 | 11.60 | 0.15 | 15.47 | 0.16 | 15.92 | 0.20 |
| 4 | 11.23 | 0.17 | 11.63 | 0.08 | 18.70 | 1.46 | 20.39 | 1.30 |
| 6 | 11.19 | 0.26 | 11.72 | 0.20 | 18.99 | 0.16 | 20.58 | 0.51 |
| 9 | 11.35 | 0.22 | 11.80 | 0.10 | 20.65 | 0.61 | 22.19 | 1.43 |

Moisture pick-up (Q)/loss (E)

| | | |
|---|---|---|
| Time | Q, Swelling Rate (n = 3) | E, Erosion Rate (n = 3) |

| (Hrs) | % Swelling | SD (%) | % Erosion | SD (%) |
|---|---|---|---|---|
| 1 | 101.75 | 3.27 | 19.36 | 1.05 |
| 2 | 163.15 | 10.96 | 20.73 | 0.52 |
| 4 | 555.97 | 51.52 | 45.00 | 3.96 |
| 6 | 577.33 | 27.99 | 40.28 | 3.22 |
| 9 | 660.60 | 129.32 | 43.95 | 10.43 |

### Tablet of Example 13

Tablet size Change

| | Initial Tablet Dimensions | | | | Final Tablet Dimensions | | | |
|---|---|---|---|---|---|---|---|---|
| | (mm) n = 3 | | | | (mm) n = 3 | | | |
| Time | Mean | SD | Mean | SD | Mean | SD | Mean | SD |
| (Hrs) | Length | (%) | Width | (%) | Length | (%) | Width | (%) |
| 1 | 11.08 | 0.18 | 11.71 | 0.22 | 14.34 | 0.06 | 14.77 | 0.10 |
| 2 | 11.28 | 0.13 | 11.87 | 0.16 | 15.82 | 0.21 | 16.24 | 0.18 |
| 4 | 11.36 | 0.04 | 11.82 | 0.34 | 18.39 | 0.79 | 19.28 | 0.70 |
| 6 | 11.32 | 0.15 | 11.96 | 0.18 | 19.07 | 0.12 | 20.73 | 0.41 |
| 9 | 11.15 | 0.14 | 11.83 | 0.08 | 22.48 | 0.19 | 23.07 | 0.41 |

Moisture pick-up (Q)/loss (E)

| | | |
|---|---|---|
| Time | Q, Swelling Rate (n = 3) | E, Erosion Rate (n = 3) |

| (Hrs) | % Swelling | SD (%) | % Erosion | SD (%) |
|---|---|---|---|---|
| 1 | 123.22 | 8.39 | 16.11 | 1.02 |
| 2 | 155.05 | 7.86 | 15.59 | 0.64 |
| 4 | 320.49 | 14.93 | 18.20 | 3.17 |
| 6 | 380.89 | 35.04 | 20.60 | 5.77 |
| 9 | 530.55 | 36.43 | 19.77 | 3.95 |

### Tablet of Example 14

Tablet size Change

| | Initial Tablet Dimensions | | | | Final Tablet Dimensions | | | |
|---|---|---|---|---|---|---|---|---|
| | (mm) from n = 3 | | | | (mm) n = 3 | | | |
| Time | Mean | SD | Mean | SD | Mean | SD | Mean | SD |
| (Hrs) | Length | (%) | Width | (%) | Length | (%) | Width | (%) |
| 1 | 11.13 | 0.10 | 11.87 | 0.09 | 14.14 | 0.05 | 14.79 | 0.11 |
| 2 | 11.14 | 0.03 | 11.89 | 0.17 | 14.12 | 0.03 | 15.19 | 0.09 |
| 4 | 11.27 | 0.10 | 11.89 | 0.10 | 15.26 | 0.29 | 16.42 | 0.56 |
| 6 | 11.07 | 0.03 | 11.92 | 0.15 | 15.31 | 0.46 | 16.47 | 0.25 |
| 9 | 11.12 | 0.13 | 11.97 | 0.16 | 17.90 | 0.45 | 20.38 | 1.27 |

Moisture pick-up (Q)/loss (E)

| | | |
|---|---|---|
| Time | Q, Swelling Rate (n = 3) | E, Erosion Rate (n = 3) |

| (Hrs) | % Swelling | SD (%) | % Erosion | SD (%) |
|---|---|---|---|---|
| 1 | 93.23 | 1.67 | 19.26 | 0.77 |
| 2 | 105.39 | 4.26 | 20.77 | 1.27 |
| 4 | 206.49 | 20.43 | 35.04 | 1.33 |
| 6 | 244.02 | 48.94 | 45.16 | 4.74 |
| 9 | 483.41 | 122.07 | 48.40 | 2.84 |

These results are summarized below:

| | | Example | Example | Example |
|---|---|---|---|---|
| | Time | 12 | 13 | 14 |
| Dimension Change | 1 h | 124%, 125% | 126%, 129% | 125%, 127% |
| (length, width; | 9 h | 161%, 170% | 195%, 200% | 182%, 188% |
| mm/mm %) | | | | |
| Moisture uptake | 9 h | 660% | 550% | 483% |
| (w/w %) | 1 h | 19% | 16% | 19% |
| Tablet Erosion | 9 h | 44% | 20% | 48% |
| (w/w %) | | | | |

All three formulations increased in dimension quickly, approximately 25% in all dimensions in 1 hour and almost doubling in length and width in 9 hours. Note that a slower erosion rate corresponds to a stronger mechanical strength (even after swelling).

### Example 60

### Floating Properties

Floating behavior studies were carried out as described below on the tablets prepared in examples 12, 13, 14, 15 and 16 according to previously described methods (see Rahman et al. Acta Pharm. 2006, 56, 49-57). Cifran® OD (a floating matrix tablet with sodium bicarbonate; see U.S. Pat. No. 6,261,601) was used for comparison purposes; however 1 out of 3 Cifran® OD tablets failed to float. Other marketed gastroretentive dosage forms, Glumetza® and Proquin® were also tested and likewise failed to float.

Floating behavior studies were carried out in a USP 23 paddle apparatus 2 (see United States Pharmacopoeia 23, US Pharmacopoeial Convention, Rockville 1993, p. 951) at a paddle speed of 50 rpm in 900 mL SGF (pH 1.2, no enzyme) at 37±0.2° C. for 24 h. The time required to rise upwards and float on the surface (floating lag time) and floating duration were determined. The floating properties of other compositions described and claimed herein may be determined using the method described herein or similar methods known to those of skill in the art.

Observations were as follows:

| Formulation | Floating Observation |
|---|---|
| Cifran ®* | Lag Time: 47 sec |
| | Two of three tablets floating after the lag time |
| | Tablets remained floating up to 8 hr of observation |
| Example 12 | Lag Time: 36 sec |
| | Three of three tablets floating after the lag time |
| | Tablets remained floating up to 8 hr of observation |
| Example 13 | Lag Time: 51 sec |
| | Three of three tablets floating after the lag time |
| | All tablets settled within 5 min; one tablet re- |
| | floated after 3 hours |
| Example 14 | No Floating |
| Example 15 | Lag Time: <2 min (n = 3) |
| | Three of three tablets floating after the lag time |
| | Tablets remained floating up to 12 hr of observation |
| Example 16 | Lag Time: <2 min (n = 3) |
| | Three of three tablets floating after the lag time |
| | Tablets remained floating up to 12 hr of observation |
| Glumetza | No Floating |
| Proquin | No Floating |

*Cifran ® OD (ciprofloxacin 500 mg tablet purchased from Ranbaxy)

Note that example 13 flotation behavior was irregular. The tablets floated within one hour, and then slowly settled or oscillated up and down without floating. After 3 hours, one tablet re-floated.

### Example 61

### Bioadhesiveness Test

Preparation of Simulated Gastric Fluid (SGF): Simulated gastric fluid (SGF) was prepared using the USP/NF procedure, as follows. Sodium chloride (2.0 g), pepsin (3.2 g; 800 to 2500 units per mg of protein.) and hydrochloric acid (7.0 mL) were dissolved in deionized water (˜987.8 mL, Emerson Resources, Inc internal system). The solution was spun for 1-2 hours or until the pepsin was fully dissolved. A measured amount of SGF solution is taken and the pH adjusted to 4-4.5, using sodium hydroxide (2N), to simulate tablets swelling in a stomach full of food.

Preparation of Tablets: Tablets were placed on their concave side in a shallow non-stick dish on a level surface. Simulated gastric fluid (pH ˜1.1 or 4-4.5), was added until the belly band (sidewall) of the tablet was half covered, (i.e. half of the tablet should be submerged in SGF solution to give the best swelling results and leave a solid upper tablet face for adhesion to the probe). The tablets were submerged for approx. 2 hours, (times varied slightly depending on tablet type), to allow swelling and disintegration of tablet, and expose the tablet core for adhesion testing. The tablets were removed using the top probe of the Texture Analyzer, with double sided sticky tape, by pushing down on the dry top of the tablet and pulling up. Alternatively, the tablets were removed with forceps, (being careful not to damage swollen section) and attached to the top probe of the Texture Analyzer using adhesive tape. The top probe was screwed onto the Texture analyzer. 7-10 tablets were used for each test. Glumetza® (500 mg metformin/tablet) was tested for comparison purposes.

Preparation of stomach tissues: cleaned, refrigerated porcine stomach (Sierra Medical) was allowed to warm to room temperature. The stomach was cut using the sphincter opening to expose internal membrane. Long strips were cut into portions large enough to cover the top of the film test rig, (sections must be larger enough to cover the 28 mm opening of the bottom stationary probe). The stomach sections were submerged in SGF for at least 15 minutes, allowing them to become wetted and thus simulating the stomach environment during testing.

Set-up of Texture Analyzer Equipment: A TA-108S mini test rig apparatus (with modifications of top metal rings) was used. The spacer plate (aluminum) was placed onto the 4 threaded posts, followed by the solid plate so as to cover the hole and create a solid back-drop for pressure resistance. The porcine stomach sample, prepared as described above, was placed on top of solid plate and push down through the 4 threaded posts to secure the sample. A large ‘O’ ring plate was place onto the posts and down on top of the tissue sample. All three plates and the tissue sample were then secured by screwing the nuts onto the four threaded posts ensuring that the tissue sample was held securely onto ‘O’ ring plate. Note that for best results, the tissue should be stretched tightly under the retainer ring.

Test Method and Parameters: A TA-XT2i texture analyzer, using Texture Exponent 32 Texture Analyzer software (“Bio-adhesion test” method selected) was used to determine bioadhesiveness. The pre-swelled test tablets were attached to the top probe and the porcine stomach membrane secured to the bottom probe. The tablets were then lowered onto the membrane at a 2 mm/s test speed. A constant force of 1 kg was applied to the tablet/membrane interface for 30 seconds through the top probe with tablet affixed. The top probe (with tablet) was then slowly removed at a post-test speed of 0.75 mm/s, until the tablet is completely removed from the membrane and shows a force of 0 g. Graphs of areas under force/distance tension were plotted, and used to calculate tensile work used to remove tablet from membrane.

The detachment force power for examples 12, 13 and 14 (n=7-10) was measured (nJ) and the results shown below. The bioadhesive properties of other compositions described and claimed herein may be determined using the method described herein or similar methods known to those of skill in the art.

### Example 12

| | |
|---|---|
| pH 1.0 | pH 4.0 |

| | | | |
|---|---|---|---|
| | 0.559 | | 1.256 |
| | 0.326 | | 0.729 |
| | 0.327 | | 0.287 |
| | 1.032 | | 1.013 |
| | 0.644 | | 1.718 |
| | 0.298 | | 0.457 |
| | 0.546 | | 1.113 |
| | 0.605 | | 1.012 |
| | 0.209 | | |
| Av. | 0.505 | Av. | 0.948 |
| STD | 0.251 | STD | 0.455 |
| RSD | 49.786 | RSD | 47.994 |
| nJ | 505028.2 | nJ | 947970.0 |

### Example 13

| | |
|---|---|
| pH 1.0 | pH 4.0 |

| | | | |
|---|---|---|---|
| | 0.555 | | 0.331 |
| | 0.388 | | 1.051 |
| | 1.378 | | 0.966 |
| | 1.415 | | 0.728 |
| | 0.835 | | 1.112 |
| | 0.492 | | 0.806 |
| | 0.611 | | 0.728 |
| | 0.488 | | 0.711 |
| | 0.518 | | 0.656 |
| Av. | 0.742 | Av. | 0.788 |
| STD | 0.391 | STD | 0.236 |
| RSD | 52.632 | RSD | 29.955 |
| nJ | 742204.7 | nJ | 787524.8 |

### Example 14

| | |
|---|---|
| pH 1.0 | pH 4.0 |

| | | | |
|---|---|---|---|
| | 0.173 | | 0.243 |
| | 0.718 | | 0.187 |
| | 0.530 | | 0.149 |
| | 0.317 | | 0.304 |
| | 0.143 | | 0.235 |
| | 1.172 | | 0.598 |
| | 0.636 | | 0.327 |
| | 0.286 | | 0.252 |
| | 0.328 | | 0.279 |
| | | | 0.248 |
| Av. | 0.478 | Av. | 0.282 |
| STD | 0.327 | STD | 0.122 |
| RSD | 68.487 | RSD | 43.369 |
| nJ | 477967.8 | nJ | 282151.1 |

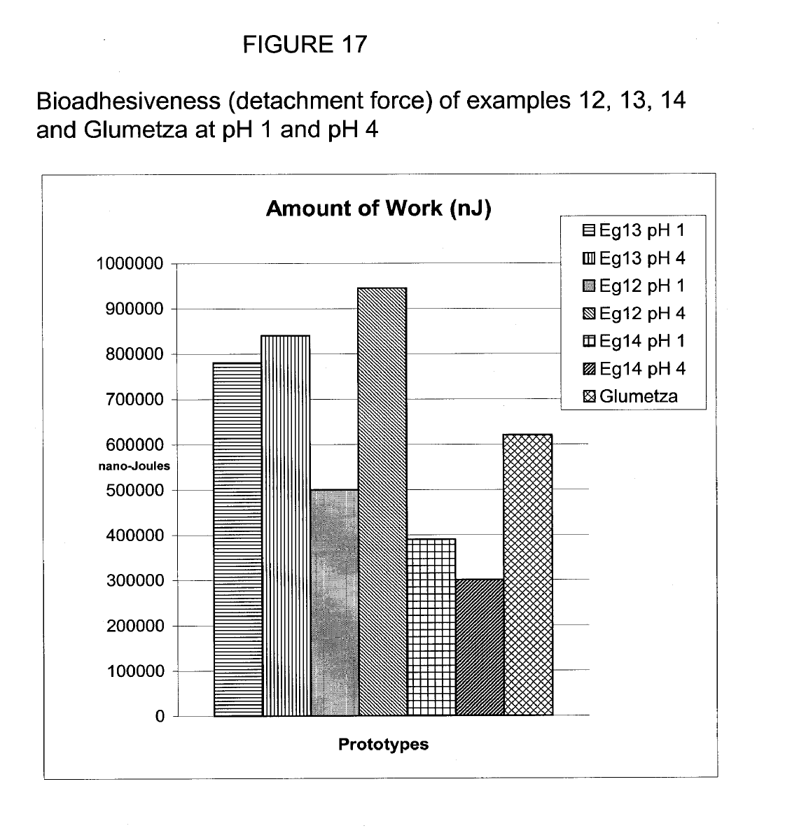

FIG. 17 shows a comparison of the three tablets tested (those from examples 12, 13 and 14) and comparator drug, Glumetza®, at pH 1 and pH 4.

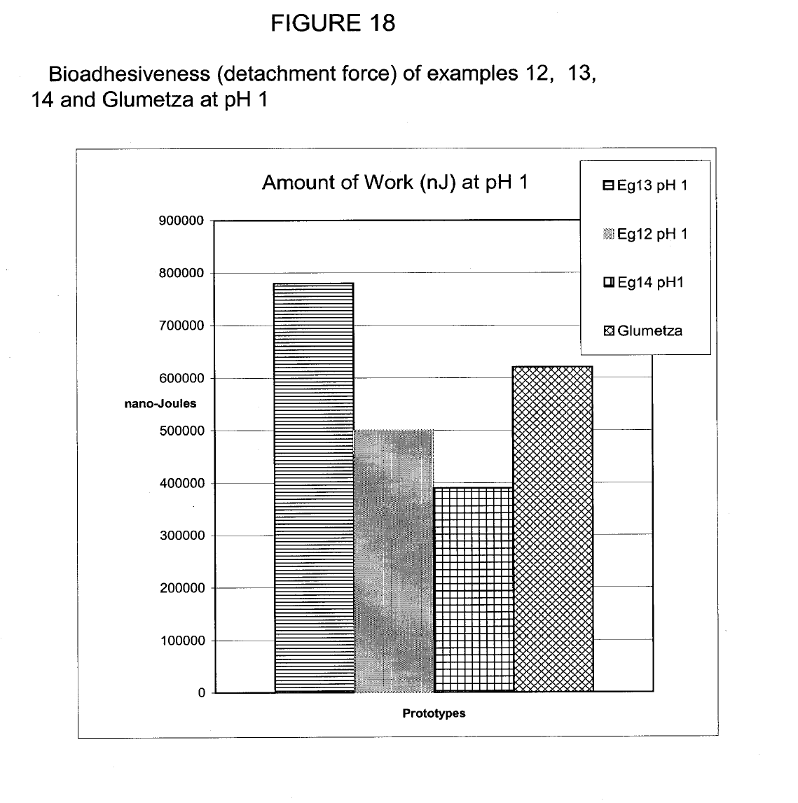

The bioadhesiveness trend at pH 1 (see FIG. 18) was:
Ex. 13>Glumetza®>Ex. 12>Ex. 14
   The bioadhesiveness trend at pH 4 was:
Ex. 12>Ex. 13>Ex. 14

The tablets of examples 12 and 13, (19% carbopol; 8% HPC; and 25% carbopol; 8% HPC, respectively) showed stronger bioadhesiveness than those of example 14, (0% carbopol; 14% HPC).

### Formulation Development Conclusions

Thus the formulations of examples 13 and 14 showed dissolution profiles of >80% drug release at 12-16 hours.
a) Nature of polymer: Carbopol 71G and HPMC gave similar release profiles at the same concentration when same excipients were used.
b) HPC (Klucel-H): gave the slowest release of methotrexate from a matrix tablet when the same excipients were used. A mixture of Carbopol and HPC or HPC alone gave a zero-order release that can be controlled by changing diluent or the polymer concentration.
c) Water-soluble diluents such as lactose and maltodextrin gave faster drug release than water-insoluble diluent dicalcium phosphate. Maltodextrin based tablets released drug slightly faster (6 hr vs. 8 hr) than the lactose-based tablet when all other components were the same.
d) Sodium bicarbonate: used in higher concentrations (>20%) used with a water-soluble diluent, acts as an effervescent tablet and breaks up the matrix tablet. When the same level was used with dicalcium phosphate, the release was slower while the tablet floated during the dissolution.

A summary of the in-vitro properties of the formulations of examples 12, 13, 14, 15 and 16 is shown in the table below.

| In-vitro test | Ex 12 | Ex 13 | Ex 14 | Ex 15 | Ex 16 |
|---|---|---|---|---|---|
| Dissolution | + | ++ | ++ | + | ++ |
| | (6 hr) | (12 hr) | (16 hr) | (18 hr) | (16 hr) |
| Swelling-size | + | ++ | ++ | ++ | ++ |
| Erosion-weight | + | ++ | + | + | + |
| Floating | ++ | + | − | ++ | ++ |
| Bioadhesiveness | ++ | +++ | + | + | + |

Among the formulations evaluated, examples 13 and 16 demonstrated desirable features for MTX-GR/SR formulations based on the in-vitro test results as summarized below:
In-vitro dissolution (USP method, pH=1): >80% drug release at 12 hour
Swelling/Erosion test: the dimension of the tablet increased 25% within 1 hour of immersion into SGF, making the tablet over 14 mm in all sides which is too large to pass through pylori. The erosion rate was low at <20% of the swollen tablet rate, making this prototype the most mechanically strong form among 14 prototypes evaluated so far.
Floating test: lag time of <3 min.
Bioadhesiveness: detachment forces of 742204.7 nJ (pH=1) and 787524.8 nJ (pH=4)

### Example 62

### In Vivo Pharmacokinetic Properties

Cohorts of six, (preferably naïve) beagle dogs (3 female/3 male; 10-12 kg) are dosed with one 5 mg Gastroretentive/SR MTX tablet or one Gastroretentive/SR methotrexate/folic acid combo tablet or control, 30 min after a standard meal. Ten 1 mL blood samples are collected at appropriate intervals extending to 72 hours. Plasma is collected after centrifugation for 10 min at 3,000 rpm at 4° C. Samples are stored frozen at −20° C. until analyzed. Serum methotrexate is determined by LC/MS/MS. Turbulent flow chromatography using a 2300 HTLC™ system (Cohesive Technologies, Franklin, Mass.) is coupled to tandem-mass spectrometry (MS/MS) performed on a triple stage quadropole from Perkin Elmer SCIEX API 365 (Sciex, Concord, Ontario, Canada) with an atmospheric pressure ionization (API) chamber. Samples may also be analyzed by HPLC. Pharmacokinetic parameter calculations are conducted using PK Functions for Excel software and statistical analyses performed using SPSS® Version 13.0. Graphs of plasma concentration versus time are plotted for each dog and the following pharmacokinetic parameters are calculated:

maximum observed concentration (Cₘₐₓ);

time at which Cₘₐₓ was observed (Tₘₐₓ);

area under the plasma concentration versus time curve (AUC) carried out to 72 hrs; and

time at which half of the methotrexate has been absorbed (t½).

### Example 63

### Clinical Study: Sustained Release Methotrexate Therapy in Patients Naïve to Methotrexate

Forty-eight adult patients naïve to methotrexate are enrolled in a prospective, longitudinal dose escalation study. A sustained release methotrexate formulation, as described herein, comprising 7.5 mg methotrexate is administered once a week and the dose increased, as appropriate to control disease activity. Standard methotrexate formulations are used as controls. Therapeutic response is assessed based on change in disease activity score (DAS28). Treating physicians are blinded to red blood cell (RBC) methotrexate levels.

### Example 64

### Clinical Study: Sustained Release Methotrexate/Folate Therapy in Patients Naïve to Methotrexate

Forty-eight adult patients naïve to methotrexate are enrolled in a prospective, longitudinal dose escalation study. A sustained release methotrexate/folate formulation, as described herein, comprising 7.5 mg methotrexate is administered once a week and the dose increased, as appropriate to control disease activity. Standard methotrexate formulations are used as controls. Therapeutic response is assessed based on change in disease activity score (DAS28). Treating physicians are blinded to red blood cell (RBC) methotrexate levels.

### Example 65

### Clinical Study: Sustained Release Methotrexate Therapy in Patients Naïve to Methotrexate

Forty-eight adult patients naïve to methotrexate are enrolled in a prospective, longitudinal dose escalation study. A sustained release methotrexate formulation, as described herein, comprising 5 mg methotrexate is administered once a week and the dose increased, as appropriate to control disease activity. Standard methotrexate formulations are used as controls. Therapeutic response is assessed based on change in disease activity score (DAS28). Treating physicians are blinded to red blood cell (RBC) methotrexate levels.

### Example 66

### Clinical Study: Sustained Release Methotrexate/Folate Therapy in Patients Naïve to Methotrexate

Forty-eight adult patients naïve to methotrexate are enrolled in a prospective, longitudinal dose escalation study. A sustained release methotrexate/folate formulation, as described herein, comprising 5 mg methotrexate is administered once a week and the dose increased, as appropriate to control disease activity. Standard methotrexate formulations are used as controls. Therapeutic response is assessed based on change in disease activity score (DAS28). Treating physicians are blinded to red blood cell (RBC) methotrexate levels.

### Example 67

### Clinical Study—Comparison of Gastroretentive MTX-SR Formulation with MTX-IR Formulation in Patients Naïve to Methotrexate

20 patients (age >18 years) with rheumatoid arthritis, naïve to MTX are enrolled for the study. Patients are randomized (2-way double blinded crossover) to receive immediate release MTX (one 5 mg tablet) or sustained release MTX (one 5 mg tablet), with a two week washout period. In addition to the appropriate enrollment criteria, patients are screened for folate and vitamin B12 deficiency. After an overnight fast, patients receive a standardized breakfast and take the MTX-SR or the MTX-IR.
Blood sampling: Blood samples 5 ml on EDTA are drawn at 0 h, 0.5 h, 1 h, 2 h, 3 h, 4 h, 6 h, 8 h, 12 h following administration and subsequently at 24 h, 48 h, 72 h, 96 h to evaluate the terminal half life of the MTX SR versus IR. Samples are processed on site. Plasma and erythrocytes, are isolated and stored.
Urine sampling: urine is collected for 48 hours following administration.
Analytes measured: Plasma MTX and 7-hydroxy MTX are measured at each time point using standard chromatographic techniques with post column photo-oxidation and fluorimetric detection. Homocysteine concentration as well as plasma endogenous purines and pyrimidine are measured by standard techniques.
PK parameters: The following pharmacokinetic parameters are calculated: AUC, Cₘₐₓ, Tₘₐₓ, t_{1/2}, urinary excretion using PK modeling using non linear regression (WINONLIN).

### Example 68

### Clinical Study—Comparison of Gastroretentive MTX-SR Formulation with MTX-IR Formulation in Patients Naïve to Methotrexate

150 patients (age >18 years) per arm, with early rheumatoid arthritis, naïve to MTX are enrolled for the study, in one or more centers. (Note, 150 patients per arm are required to demonstrate a significant (p<0.05; with 80% power) reduction in the incidence of nausea from 25% to 12.5%.) The study is a 24 week dose escalation study, where patients are randomized (2-way double blinded crossover) to receive doses as follows:
Weeks 1-4: 5 mg MTX SR or 5 mg IR
Weeks 5-8: 10 mg MTX SR or 10 mg IR
Weeks 9-24: 15 mg MTX SR of 15 mg IR.
   Patients are assessed monthly for a total of 6 months. At each monthly visit patients are assessed for tolerability and efficacy with collection of individual components of the ACR and DAS criteria. Quality of life parameters (HAQ, EQ-SD, SF-36) are assessed to derive Quality Adjusted life years and assess the pharmacoeconomic impact of the SR versus IR formulations.

The foregoing is offered primarily for purposes of illustration. It will be readily apparent to those skilled in the art that further drugs can be included, and that the shapes, components, additives, proportions, methods of formulation, and other parameters described herein can be modified further or substituted in various ways without departing from the spirit and scope of the invention.

What is claimed is:

1. A method for treating an autoimmune disease in a subject comprising administering to the subject an oral pharmaceutical composition comprising methotrexate, wherein the pharmaceutical composition releases at least some of the methotrexate into the upper gastrointestinal tract of the subject over a sustained period of time and half of the total systemic methotrexate AUC is delivered between about 4 and about 24 hours.

2. The method of claim 1, wherein the autoimmune disease is selected from ankylosing spondylitis, Crohn's disease, rheumatoid arthritis, juvenile rheumatoid arthritis, psoriatic arthritis, psoriasis, scleroderma, polymyositis, lupus, systemic lupus erythematosus, vasculitis, inflammatory bowel disease, Sjogren's syndrome and multiple sclerosis.

3. The method of claim 1, wherein the autoimmune disease is rheumatoid arthritis.

4. The method of claim 1, wherein the pharmaceutical composition is a monolithic solid.

5. The method of claim 1, wherein upon administration to a fed subject, the pharmaceutical composition remains in the stomach for between about 6 to about 10 hours.

6. The method of claim 1, wherein the methotrexate is present in the pharmaceutical composition in an amount of about 5 mg and wherein after oral administration of the pharmaceutical composition to a fed subject, the composition exhibits (i) a methotrexate Cₘₐₓ of between about 50 and about 250 nmol/ml, and (ii) half of the total systemic methotrexate AUC is delivered between about 4 and about 10 hours.

7. The method of claim 1, wherein the methotrexate is present in the pharmaceutical composition in an amount of about 10 mg and wherein after oral administration of the pharmaceutical composition to a fed subject, the composition exhibits (i) a methotrexate Cₘₐₓ of between about 100 and about 500 nmol/ml, and (ii) half of the total systemic methotrexate AUC is delivered between about 4 and about 10 hours.

8. The method of claim 1, wherein the methotrexate is present in the pharmaceutical composition in an amount of about 15 mg and wherein after oral administration of the pharmaceutical composition to a fed subject, the composition exhibits (i) a methotrexate Cₘₐₓ of between about 150 and about 750 nmol/ml, and (ii) half of the total systemic methotrexate AUC is delivered between about 4 and about 10 hours.

9. The method of claim 1, wherein the methotrexate is present in the pharmaceutical composition in an amount of about 20 mg and wherein after oral administration of the pharmaceutical composition to a fed subject, the composition exhibits (i) a methotrexate Cₘₐₓ of between about 200 and about 1000 nmol/ml, and (ii) half of the total systemic methotrexate AUC is delivered between about 4 and about 10 hours.

10. A method for treating or preventing cancer in a subject comprising administering to the subject an oral pharmaceutical composition comprising methotrexate, wherein the pharmaceutical composition is a monolithic solid and releases the methotrexate into the upper gastrointestinal tract of the subject over a sustained period of time and half of the total systemic methotrexate AUC is delivered between about 4 and about 24 hours.

11. The method of claim 10, wherein the cancer is selected from acute lymphocytic leukemia, meningeal leukemia, choriocarcinoma, osteosarcoma, cutaneous lymphoma, Burkitt's lymphoma, non-Hodgkin's lymphoma, breast cancer, head and neck cancer, ovarian cancer and bladder cancer.

12. The method of claim 10, wherein the pharmaceutical composition is a monolithic solid.

13. A method for optimizing therapeutic efficacy of methotrexate for treatment of an autoimmune disease in a subject comprising administering to the subject an oral pharmaceutical composition comprising methotrexate, wherein the pharmaceutical composition releases at least some of the methotrexate into the upper gastrointestinal tract of the subject over a sustained period of time and half of the total systemic methotrexate AUC is delivered between about 4 and about 24 hours.

14. The method of claim 13, upon administration of the pharmaceutical composition to a group of patients, the methotrexate in the sustained release formulation is at least about 10% more bioavailable than Trexall® or Rheumatrex.

15. A method for reducing the toxicity of methotrexate for treatment of an autoimmune disease in a subject comprising administering to the subject an oral pharmaceutical composition comprising methotrexate, wherein the pharmaceutical composition releases at least some of the methotrexate into the upper gastrointestinal tract of the subject over a sustained period of time and half of the total systemic methotrexate AUC is delivered between about 4 and about 24 hours.

16. A method for improving the risk/benefit ratio of methotrexate for treatment of an autoimmune disease in a subject comprising administering to the subject an oral pharmaceutical composition comprising methotrexate, wherein the pharmaceutical composition releases at least some of the methotrexate into the upper gastrointestinal tract of the subject over a sustained period of time and half of the total systemic methotrexate AUC is delivered between about 4 and about 24 hours.

17. An oral pharmaceutical composition comprising methotrexate, wherein the pharmaceutical composition exhibits a methotrexate release rate of greater than about 80% within about 8 to about 18 hours as measured by the USP Type II dissolution apparatus (paddle method) at 100 rpm in 900 ml 0.1N hydrochloric acid at 37° C. using Methotrexate USP with UV detection at 302 mm.

18. The pharmaceutical composition of claim 17, wherein the pharmaceutical composition is in the form of a monolithic solid.

19. The pharmaceutical composition of claim 17, further comprising a hydrophilic polymer.

20. The pharmaceutical composition of claim 19, wherein the hydrophilic polymer comprises carbopol, hydroxypropyl cellulose, hydroxymethyl cellulose, polyethylene oxide, or mixtures thereof.

21. The pharmaceutical composition of claim 19, wherein the hydrophilic polymer is carbopol.

22. The pharmaceutical composition of claim 19, wherein the hydrophilic polymer makes up about 20 wt-% to about 40 wt-% of the composition.

23. The pharmaceutical composition of claim 17, wherein the methotrexate is present in an amount of about 2 mg to about 15 mg.

24. The pharmaceutical composition of claim 21, wherein the carbopol makes up about 20 wt-% to about 30 wt-% of the pharmaceutical composition and the methotrexate is present in an amount of about 2 mg to about 15 mg.

25. The pharmaceutical composition of claim 21, further comprising at least one gas generating agent, wherein the gas generating agent is a carbonate or bicarbonate salt of a Group I or Group II metal.

26. The pharmaceutical composition of claim 25, wherein the gas generating agent is sodium bicarbonate.

27. The pharmaceutical composition of claim 25, wherein the carbonate or bicarbonate salt of a Group I or Group II metal makes up about 3 wt-% to about 20 wt-% of the pharmaceutical composition.

28. The pharmaceutical composition of claim 18, wherein the monolithic solid tablet is a diamond or triagonal biplanar shaped tablet with a triangular lateral length of 8-14 mm and a vertical axis length of about 4-9 mm.

29. The pharmaceutical composition of claim 17, wherein the pharmaceutical composition floats within 20 minutes after immersion in 900 mL of simulated gastric fluid at pH 1.2 and at 37° C.

30. The pharmaceutical composition of claim 29, wherein the pharmaceutical composition remains floating for at least 8 hours after immersion in the simulated gastric fluid.

31. The pharmaceutical composition of claim 17, wherein the pharmaceutical composition adheres to intestinal tissue with a force of at least 500,000 nJ.

32. The pharmaceutical composition of claim 17, wherein upon immersion in simulated gastric fluid at pH 1.2 and at 37° C., the pharmaceutical composition swells in size by at least about 25% in all measurements within about 1 hour.

33. The pharmaceutical composition of claim 32, wherein the composition swells in size by at least about 50% in at least one measurement within about 1 hour and about 9 hours after immersion in the simulated gastric fluid.
